(19) United States
(12) Patent Application Publication
(10) Pub. No.: US 2004/0002476 A1
(43) Pub. Date: Jan. 1, 2004

(54) Modified fluorinated nucleoside analogues

(76) Inventors: Lieven J. Stuyver, Snellville, GA (US); Junxing Shi, Duluth, GA (US); Kyoichi A. Watanabe, Stone Mountain, GA (US)

Correspondence Address: KING & SPALDING, 191 PEACHTREE STREET, N.E., ATLANTA, GA, 30303-1763, US

(21) Appl. No.: 10/366,144

(22) Filed: Feb. 13, 2003

Related U.S. Application Data
(60) Provisional application No. 60/357,411, filed on Feb. 14, 2002.
(60) Provisional application No. 60/358,140, filed on Feb. 20, 2002.

(51) Int. Cl. A61K 31/7076; A61K 31/7072; A61K 31/522; A61K 31/513; C07H 19/073; C07H 19/06; C07H 19/067; C07D 473/14; C 07D 4 5/02

(52) U.S. Cl. 514/49; 514/263.23; 514/269; 536/28.5; 544/276; 544/318

(57) ABSTRACT

The invention is a compound, composition, use for and a method of treating Flaviviridae (Hepacivirus, Flavirius, Pestivirus) infections, including BVDV and HCV, or abnormal cellular proliferation, including malignant tumors, in a host including animals, and especially humans, using a β-D or β-L nucleoside of general formula (I)-(XX), or their pharmaceutically acceptable salt or prodrug thereof.

This application claims priority to U.S. provisional application No. 60/357,411, filed on Feb. 14, 2002, and U.S. Ser. No. 60/358,140, filed on Feb. 20, 2002.

### FIELD OF THE INVENTION

The present invention includes compounds and methods for the treatment of Flaviviridae infections, such as bovine viral diarrhea virus (“BVDV”), Dengue Virus (DENV), West Nile Virus (WNV) and hepatitis C virus (HCV) as well as abnormal cellular proliferation.

### BACKGROUND OF THE INVENTION

Flavirididae

The Flaviviridae is a group of positive single-stranded RNA viruses with a genome size from 9-15 kb. They are enveloped viruses of approximately 40-50 nm. An overview of the Flaviviridae taxonomy is available from the International Committee for Taxonomy of Viruses. The Flaviviridae consists of three genera.

1. Flaviviruses. This genus includes the Dengue virus group (Dengue virus, Dengue virus type 1, Dengue virus type 2, Dengue virus type 3, Dengue virus type 4), the Japanese encephalitis virus group (Alfuy Virus, Japanese encephalitis virus, Kookaburra virus, Koutango virus, Kunjin virus, Murray Valley encephalitis virus, St. Louis encephalitis virus, Stratford virus, Usutu virus, West Nile Virus), the Modoc virus group, the Rio Bravo virus group (Apoi virus, Rio Brovo virus, Saboya virus), the Ntaya virus group, the Tick-Borne encephalitis group (tick born encephalitis virus), the Tyuleniy virus group, Uganda S virus group and the Yellow Fever virus group. Apart from these major groups, there are some additional Flaviviruses that are unclassified.

2. Pestiviruses. This genus includes Bovine Viral Diarrhea Virus-2 (BVDV-2), Pestivirus type 1 (including BVDV), Pestivirus type 2 (including Hog Cholera Virus) and Pestivirus type 3 (including Border Disease Virus).

3. Hepaciviruses. This genus contains only one species, the Hepatitis C virus (HCV), which is composed of many clades, types and subtypes.

One of the most important Flaviviridae infections in humans is caused by the hepatitis C virus (HCV). This is the second major cause of viral hepatitis, with an estimated 170 million carriers world-wide (World Health Organization; Hepatitis C: global prevalence, Weekly Epidemiological Record, 1997, 72, 341), 3.9 million of whom reside in the United States (Centers for Disease Control; unpublished data, http://www.cdc.gov/ncidod/diseases/hepatitis/heptab3.htm). Chronic infection with HCV can lead to liver inflammation, cirrhosis, cancer and death.

The genomic organization of the Flaviviridae share many common features. The hepatitis C virus (HCV) genome is often used as a model. HCV is a small, enveloped virus with a positive single-stranded RNA genome of 9.6 kb within the nucleocapsid. The genome contains a single open reading frame (ORF) encoding a polyprotein of just over 3,000 amino acids, which is cleaved to generate the mature structural and nonstructural viral proteins. The ORF is flanked by 5′ and 3′ non-translated regions (NTRs) of a few hundred nucleotides in length, which are important for RNA translation and replication. The translated polyprotein contains the structural core (C) and envelope proteins (E1, E2, p7) at the N-terminus, followed by the nonstructural proteins (NS2, NS3, NS4A, NS4B, NS5A, NS5B). The mature structural proteins are generated via cleavage by the host signal peptidase (see: Hijikata, M. et al. Proc. Nat. Acad. Sci., USA, 1991, 88, 5547; Hussy, P. et al. Virology, 1996, 224, 93; Lin, C. et al. J. Virol., 1994, 68, 5063; Mizushima, H. et al. J. Virol., 1994, 68, 2731; Mizushima, H. et al. J. Virol., 1994, 68, 6215; Santolini, E. et al. J. Virol., 1994, 68, 3631; Selby, M. J. et al. Virology, 1994, 204, 114; and Grakoui, A. et al. Proc. Nat. Acad. Sci., USA, 1993, 90, 10538). The junction between NS2 and NS3 is autocatalytically cleaved by the NS2/NS3 protease (see: Hijikata, M. et al. J. Virol., 1993, 67, 4665 and Bartenschlager, R. et al. J. Virol., 1994, 68, 5045), while the remaining four junctions are cleaved by the N-terminal serine protease domain of NS3 complexed with NS4A (see: Failla, C. et al. J. Virol., 1994, 68, 3753; Lin, C. et al. J. Virol., 1994, 68, 8147; Tanji, Y. et al. J. Virol., 1995, 69, 1575 and Tai, C. L. et al. J. Virol., 1996, 70, 8477). The NS3 protein also contains the NTP-dependent helicase activity which unwinds duplex RNA during replication. The NS5B protein possesses RNA-dependent RNA polymerase (RDRP) activity (see: Behrens, S. E. et al. EMBO J., 1996, 15, 12; Lohmann, V. et al. J. Virol., 1997, 71, 8416-8428 and Lohmann, V. et al. Virology, 1998, 249, 108), which is essential for viral replication (Ferrari, E. et al. J. Virol., 1999, 73, 1649). It is emphasized here that, unlike HBV or HIV, no DNA is involved in the replication of HCV. Recently in vitro experiments using NS5B, substrate specificity for HCV-RDRP was studied using guanosine 5′-monophosphate (GMP), 5′-diphosphate (GDP), 5′-triphosphate (GTP) and the 5′-triphosphate of 2′-deoxy and 2′,3′-dideoxy guanosine (dGTP and ddGTP, respectively). The authors claimed that HCV-RDRP has a strict specificity for ribonucleoside 5′-triphosphates and requires the 2′- and 3′-OH groups (Lohmann; Virology, 108).

Dengue Virus (DENV) is the causative agent of Dengue Hemorrhagic Fever (DHF). According to the world Health Organization (WHO), two fifths of the world population are now at risk for infection with this virus. An estimated 500,000 cases of DHF require hospitalization each year with a mortality rate of 5% in children.

West Nile Virus (WNV), a flavivirus previously known to exist only in intertropical regions, has emerged in recent years in temperate areas of Europe and North America, presenting a threat to public health. The most serious manifestation of WNV infection is fatal encephalitis in humans. Outbreaks in New York City and sporadic occurrences in the Southern United States were reported since 1999.

Examples of antiviral agents that have been identified as active against the Flaviviridae family of viruses include:

(1) interferon and ribavirin (Battaglia, A.M. et al., Ann. Pharmacother, 2000, 34, 487-494); Berenguer, M. et al. Antivir. Ther., 1998, 3 (Suppl. 3), 125-136).

Ribavirin (1-β-D-ribofuranosyl-1-1,2,4-triazole-3-carboxamide) is a synthetic, non-interferon-inducing, broad spectrum antiviral nucleoside analog. It is sold under the trade names Virazole™ (The Merck Index, 11th edition, Editor: Budavari, S., Merck & Co., Inc., Rahway, N.J., p1304, 1989); Rebetol (Schering Plough) and Copegus (Roche). U.S. Pat. No. 3,798,209 and RE Pat. No. 29,835 disclose and claim ribavirin. Ribavirin is structurally similar to guanosine, and has in vitro activity against several DNA and RNA viruses including Flaviviridae (Gary L. Davis. Gastroenterology 118:S104-S114, 2000). U.S. Pat. No 4,211,771 (to ICN Pharmaceuticals) discloses the use of ribavirin as an antiviral agent.

Ribavirin reduces serum amino transferase levels to normal in 40% of patients, but it does not lower serum levels of HCV-RNA (Gary L. Davis. Gastroenterology 118:S104-S114, 2000). Thus, ribavirin alone is not effective in reducing viral RNA levels. Additionally, ribavirin has significant toxicity and is known to induce anemia.

Interferons (IFNs) are compounds that have been commercially available for the treatment of chronic hepatitis for nearly a decade. IFNs are glycoproteins produced by immune cells in response to viral infection. IFNs inhibit viral replication of many viruses, including HCV, and when used as the sole treatment for hepatitis C infection, IFN suppresses serum HCV-RNA to undetectable levels. Additionally, IFN normalizes serum amino transferase levels. Unfortunately, the effects of IFN are temporary and a sustained response occurs in only 8%-9% of patients chronically infected with HCV (Gary L. Davis. Gastroenterology 118:S104-S114, 2000).

A number of patents disclose HCV treatments using interferon-based therapies. For example, U.S. Pat. No. 5,980,884 to Blatt et al. discloses methods for retreatment of patients afflicted with HCV using consensus interferon. U.S. Pat. No. 5,942,223 to Bazer et al. discloses an anti-HCV therapy using ovine or bovine interferon-tau. U.S. Pat. No. 5,928,636 to Alber et al. discloses the combination therapy of interleukin-12 and interferon alpha for the treatment of infectious diseases including HCV. U.S. Pat. No. 5,908,621 to Glue et al. discloses the use of polyethylene glycol modified interferon for the treatment of HCV. U.S. Pat. No. 5,849,696 to Chretien et al. discloses the use of thymosins, alone or in combination with interferon, for treating HCV. U.S. Pat. No. 5,830,455 to Valtuena et al. discloses a combination HCV therapy employing interferon and a free radical scavenger. U.S. Pat. No. 5,738,845 to Imakawa discloses the use of human interferon tau proteins for treating HCV. Other interferon-based treatments for HCV are disclosed in U.S. Pat. No. 5,676,942 to Testa et al., U.S. Pat. No. 5,372,808 to Blatt et al., and U.S. Pat. No. 5,849,696.

Schering-Plough sells ribavirin as Rebetol® capsules (200 mg) for administration to patients with HCV. The U.S. FDA has approved Rebetol capsules to treat chronic HCV infection in combination with Schering's alpha interferon-2b products Intron® A and PEG-Intron™. Rebetol capsules are not approved for monotherapy (i.e., administration independent of Intron®A or PEG-Intron), although Intron A and PEG-Intron are approved for monotherapy (i.e., administration without ribavirin). Hoffman La Roche is selling ribavirin under the name CoPegus in Europe and the United States, also for use in combination with interferon for the treatment of HCV. Other alpha interferon products include Roferon-A (Hoffmann-La Roche), Infergen® (Intermune, formerly Amgen's product), and Wellferon® (Wellcome Foundation) are currently FDA-approved for HCV monotherapy. Interferon products currently in development for HCV include: Roferon-A (interferon alfa-2a) by Roche, PEGASYS (pegylated interferon alfa-2a) by Roche, INFERGEN (interferon alfacon-1) by InterMune, OMNIFERON (natural interferon) by Viragen, ALBUFERON by Human Genome Sciences, REBIF (interferon beta-1a) by Ares-Serono, Omega Interferon by BioMedicine, Oral Interferon Alpha by Amarillo Biosciences, and Interferon gamma-1b by InterMune.

The combination of IFN and ribavirin for the treatment of HCV infection has been reported to be effective in the treatment of IFN naïve patients (Battaglia, A. M. et al., Ann. Pharmacother. 34:487-494, 2000). Combination treatment is effective both before hepatitis develops and when histological disease is present (Berenguer, M. et al. Antivir. Ther. 3(Suppl. 3):125-136, 1998). Currently, the most effective therapy for HCV is combination therapy of pegylated interferon with ribavirin (2002 NIH Consensus Development Conference on the Management of Hepatitis C). However, the side effects of combination therapy can be significant and include hemolysis, flu-like symptoms, anemia, and fatigue (Gary L. Davis. Gastroenterology 118:S104-S114, 2000).

(2) Substrate-based NS3 protease inhibitors (Attwood et al., Antiviral peptide derivatives, PCT WO 98/22496, 1998; Attwood et al., Antiviral Chemistry and Chemotherapy 1999, 10, 259-273; Attwood et al., Preparation and use of amino acid derivatives as anti-viral agents, German Patent Pub. DE 19914474; Tung et al. Inhibitors of serine proteases, particularly hepatitis C virus NS3 protease, PCT WO 98/17679), including alphaketoamides and hydrazinoureas, and inhibitors that terminate in an electrophile such as a boronic acid or phosphonate (Llinas-Brunet et al, Hepatitis C inhibitor peptide analogues, PCT WO 99/07734).

(3) Non-substrate-based inhibitors such as 2,4,6-trihydroxy-3-nitro-benzamide derivatives (Sudo K. et al., Biochemical and Biophysical Research Communications, 1997, 238, 643-647; Sudo K. et al. Antiviral Chemistry and Chemotherapy, 1998, 9, 186), including RD3-4082 and RD3-4078, the former substituted on the amide with a 14 carbon chain and the latter processing a para-phenoxyphenyl group.

(4) Thiazolidine derivatives which show relevant inhibition in a reverse-phase HPLC assay with an NS3/4A fusion protein and NS5A/5B substrate (Sudo K. et al., Antiviral Research, 1996, 32, 9-18), especially compound RD-1-6250, possessing a fused cinnamoyl moiety substituted with a long alkyl chain, RD4 6205 and RD4 6193.

(5) Thiazolidines and benzanilides identified in Kakiuchi N. et al. J. EBS Letters 421, 217-220; Takeshita N. et al. Analytical Biochemistry, 1997, 247, 242-246.

(6) A phenan-threnequinone possessing activity against protease in a SDS-PAGE and autoradiography assay isolated from the fermentation culture broth of Streptomyces sp., Sch 68631 (Chu M. et al., Tetrahedron Letters, 1996, 37, 7229-7232), and Sch 351633, isolated from the fungus Penicillium griscofuluum, which demonstrates activity in a scintillation proximity assay (Chu M. et al., Bioorganic and Medicinal Chemistry Letters 9, 1949-1952).

(7) Selective NS3 inhibitors based on the macromolecule elgin c, isolated from leech (Qasim M. A. et al., Biochemistry, 1997, 36, 1598-1607).

(8) Helicase inhibitors (Diana G. D. et al., Compounds, compositions and methods for treatment of hepatitis C, U.S. Pat. No. 5,633,358; Diana G. D. et al., Piperidine derivatives, pharmaceutical compositions thereof and their use in the treatment of hepatitis C, PCT WO 97/36554).

(9) Polymerase inhibitors such as nucleotide analogues, gliotoxin (Ferrari R. et al. Journal of Virology, 1999, 73, 1649-1654), and the natural product cerulenin (Lohmann V. et al., Virology, 1998, 249, 108-118).

(10) Antisense phosphorothioate oligodeoxynucleotides (S-ODN) complementary to sequence stretches in the 5′ non-coding region (NCR) of the virus (Alt M. et al., Hepatology, 1995, 22, 707-717), or nucleotides 326-348 comprising the 3′ end of the NCR and nucleotides 371-388 located in the core coding region of the HCV RNA (Alt M. et al., Archives of Virology, 1997, 142, 589-599; Galderisi U. et al., Journal of Cellular Physiology, 1999, 181, 251-257).

(11) Inhibitors of IRES-dependent translation (Ikeda N et al., Agent for the prevention and treatment of hepatitis C, Japanese Patent Pub. JP-08268890; Kai Y. et al. Prevention and treatment of viral diseases, Japanese Patent Pub. JP-10101591).

(12) Nuclease-resistant ribozymes (Maccjak, D. J. et al., Hepatology 1999, 30, abstract 995).

(13) Nucleoside analogs have also been developed for the treatment of Flaviviridae infections.

Idenix Pharmaceuticals, Ltd. discloses branched nucleosides, and their use in the treatment of HCV and flaviviruses and pestiviruses in International Publication Nos. WO 01/90121 (filed May 23, 2001) and WO 01/92282 (filed May 26, 2001). A method for the treatment of hepatitis C infection (and flaviviruses and pestiviruses) in humans and other host animals is disclosed in the Idenix publications that includes administering an effective amount of a biologically active 1′, 2′, 3′ or 4′-branched β-D or β-L nucleosides or a pharmaceutically acceptable salt or prodrug thereof, administered either alone or in combination, optionally in a pharmaceutically acceptable carrier.

WO 01/96353 (filed Jun. 15, 2001) to Indenix Pharmaceuticals, Ltd. discloses 3′-prodrugs of 2′-deoxy-β-L-nucleosides for the treatment of HBV. U.S. Pat. No. 4,957,924 to Beauchamp discloses various therapeutic esters of acyclovir.

Other patent applications disclosing the use of certain nucleoside analogs to treat hepatitis C virus include: PCT/CA00/01316 (WO 01/32153; filed Nov. 3, 2000) and PCT/CA01/00197 (WO 01/60315; filed Feb. 19, 2001) filed by BioChem Pharma, Inc. (now Shire Biochem, Inc.); PCT/US02/01531 (WO 02/057425; filed Jan. 18, 2002) and PCT/US02/03086 (WO 02/057287; filed Jan. 18, 2002) filed by Merck & Co., Inc., PCT/EP01/09633 (WO 02/18404; published Aug. 21, 2001) filed by Roche, and PCT Publication No. WO 01/79246 (filed Apr. 13, 2001) and WO 02/32920 (filed Oct. 18, 2001) by Pharmasset.

(14) Other miscellaneous compounds including 1-amino-alkylcyclohexanes (U.S. Pat. No. 6,034,134 to Gold et al.), alkyl lipids (U.S. Pat. No. 5,922,757 to Chojkier et al.), vitamin E and other antioxidants (U.S. Pat. No. 5,922,757 to Chojkier et al.), squalene, amantadine, bile acids (U.S. Pat. No. 5,846,964 to Ozeki et al.), N-(phosphonoacetyl)-L-aspartic acid, (U.S. Pat. No. 5,830,905 to Diana et al.), benzenedicarboxamides (U.S. Pat. No. 5,633,388 to Diana et al.), polyadenylic acid derivatives (U.S. Pat. No. 5,496,546 to Wang et al.), 2′,3′-dideoxyinosine (U.S. Pat. No. 5,026,687 to Yarchoan et al.), and benzimidazoles (U.S. Pat. No. 5,891,874 to Colacino et al.).

(15) Other compounds currently in clinical development for treatment of hepatitis c virus include: Interleukin-10 by Schering-Plough, IP-501 by Interneuron, Merimebodib VX-497 by Vertex, AMANTADINE (Symmetrel) by Endo Labs Solvay, HEPTAZYME by RPI, IDN-6556 by Idun Pharma., XTL-002 by XTL., HCV/MF59 by Chiron, CIVACIR by NABI, LEVOVIRIN by ICN, VIRAMIDINE by ICN, ZADAXIN (thymosin alfa-1) by Sci Clone, CEPLENE (histamine dihydrochloride) by Maxim, VX 950/LY 570310 by Vertex/Eli Lilly, ISIS 14803 by Isis Pharmaceutical/Elan, IDN-6556 by Idun Pharmaceuticals, Inc. and JTK 003 by AKROS Pharma.

U.S. Pat. No. 6,348,587 to Emory University and the University of Georgia Research Foundation discloses the use of 2′-fluoronucleosides for the treatment of HIV, hepatitis B, hepatitis C and abnormal cellular proliferation.

Abnormal Cellular Proliferation

Cellular differentiation, growth, function and death are regulated by a complex network of mechanisms at the molecular level in a multicellular organism. In the healthy animal or human, these mechanisms allow the cell to carry out its designed function and then die at a programmed rate.

Abnormal cellular proliferation, notably hyperproliferation, can occur as a result of a wide variety of factors, including genetic mutation, infection, exposure to toxins, autoimmune disorders, and benign or malignant tumor induction.

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. In normal skin the time required for a cell to move from the basal layer to the upper granular layer is about five weeks. In psoriasis, this time is only 6 to 9 days, partially due to an increase in the number of proliferating cells and an increase in the proportion of cells which are dividing (G. Grove, Int. J. Dermatol. 18:111, 1979). Approximately 2% of the population in the United States have psoriasis, occurring in about 3% of Caucasian Americans, in about 1% of African Americans, and rarely in native Americans. Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma.

Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue cause, for example, restenosis, retinopathies and atherosclerosis. The advanced lesions of atherosclerosis result from an excessive inflammatory-proliferative response to an insult to the endothelium and smooth muscle of the artery wall (Ross, R. Nature, 1993, 362:801-809). Both cell migration and cell proliferation play a role in the formation of atherosclerotic lesions.

Fibrotic disorders are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

Mesangial disorders are brought about by abnormal proliferation of mesangial cells. Mesangial hyperproliferative cell disorders include various human renal diseases, such as glomerulonephritis, diabetic nephropathy, malignant nephrosclerosis, thrombotic micro-angiopathy syndromes, transplant rejection, and glomerulopathies.

Another disease with a proliferative component is rheumatoid arthritis. Rheumatoid arthritis is generally considered an autoimmune disease that is thought to be associated with activity of autoreactive T cells (See, e.g., Harris, E. D., Jr., The New England Journal of Medicine, 1990, 322: 1277-1289), and to be caused by autoantibodies produced against collagen and IgE.

Other disorders that can include an abnormal cellular proliferative component include Behcet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

A tumor, also called a neoplasm, is a new growth of tissue in which the multiplication of cells is uncontrolled and progressive. A benign tumor is one that lacks the properties of invasion and metastasis and is usually surrounded by a fibrous capsule. A malignant tumor (i.e., cancer) is one that is capable of both invasion and metastasis. Malignant tumors also show a greater degree of anaplasia (i.e., loss of differentiation of cells and of their orientation to one another and to their axial framework) than benign tumors.

Approximately 1.2 million Americans are diagnosed with cancer each year, 8,000 of which are children. In addition, 500,000 Americans die from cancer each year in the United States alone. Prostate and lung cancers are the leading causes of death in men while breast and lung cancer are the leading causes of death in women. It is estimated that cancer-related costs account for about 10 percent of the total amount spent on disease treatment in the United States (CNN.Cancer.Facts: http://www.cnn.com/HEALTH/9511/conquer_cancer/facts/index.html, page 2 of 2, Jul. 18, 1999).

In view of the severity of diseases associated with Flaviviridae infection and/or abnormally proliferating cells, including cancer, and their pervasiveness in animals, including humans, it is an object of the present invention to provide a compound, method and composition for the treatment of a host, including animals and especially humans, with a disease associated with a Flaviviridae infection and/or abnormally proliferating cells.

It is a particular object of the present invention to provide a compound, method and composition for the treatment of a host, including animals and especially humans, infected with a Flaviviridae virus.

It is a further object to provide a compound, method and composition for the treatment of a host, including animals and especially humans, infected with hepatitis C virus.

It is another object of the present invention to provide a compound, method and composition for the treatment of a host, including animals and especially humans, with abnormal cellular proliferation.

It is yet another object to provide a compound, method and composition for the treatment of a host, including animals and especially humans, with a malignant tumor.

### SUMMARY OF THE INVENTION

The present invention is a β-D or β-L nucleoside of the formula (I)-(XX), or its pharmaceutically acceptable salt or prodrug, and the use of such compounds for the treatment of a host infected with a virus belonging to the Flaviviridae family. The invention also includes a method for treating a Flaviviridae infection, including an HCV infection, that includes the administration of an anti-viral effective amount of a β-D or β-L nucleoside of the formula (I)-(XX), or its pharmaceutically acceptable salt or prodrug, optionally in a pharmaceutically acceptable carrier or diluent, optionally in combination or alternation with another effective antiviral agent.

Alternatively, a β-D or β-L nucleoside of the formula (I)-(XX), and in particular, (III)-(V) or (VIII)-(X), or its pharmaceutically acceptable salt or prodrug thereof, can be used for the treatment of abnormal cellular proliferation. The invention also includes a method for treating abnormal cellular proliferation, including a malignant tumor, that includes the administration of an anti-proliferatively effective amount of a β-D or β-L nucleoside of the formula (I)-(XX), or its pharmaceutically acceptable salt or prodrug, optionally in a pharmaceutically acceptable carrier or diluent, optionally in combination or alternation with another effective antiproliferative agent.

In one embodiment of the present invention, the nucleoside is a β-D or β-L nucleoside of the general formula (I)-(XX):

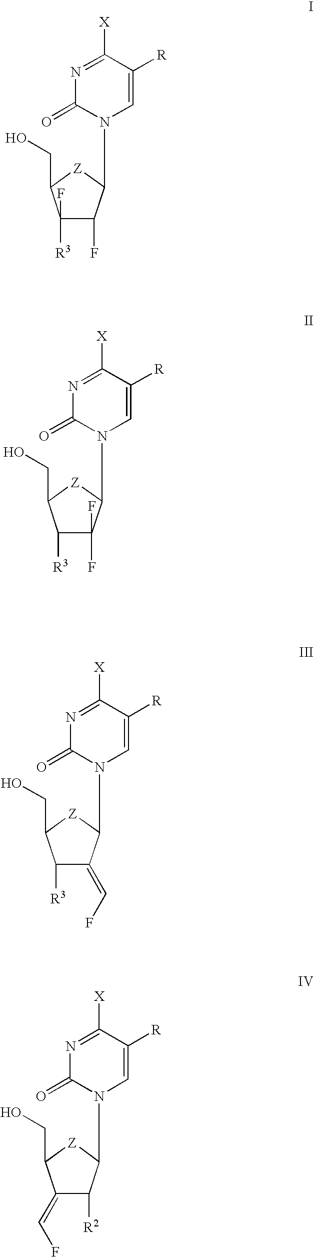

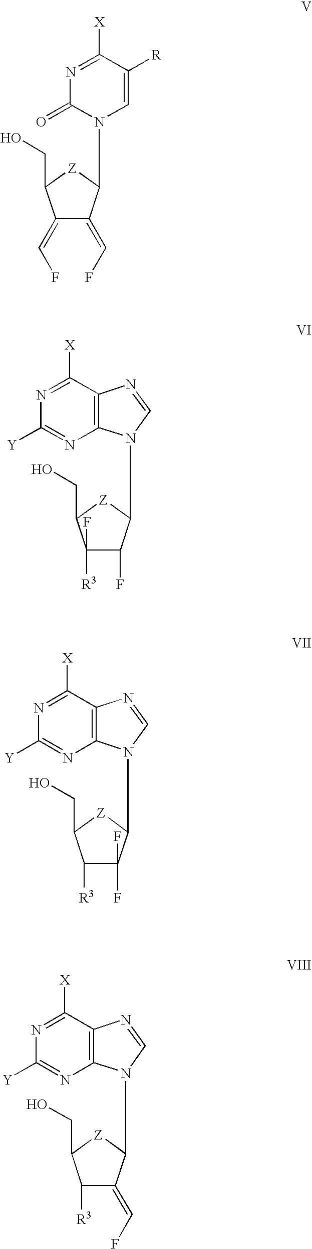

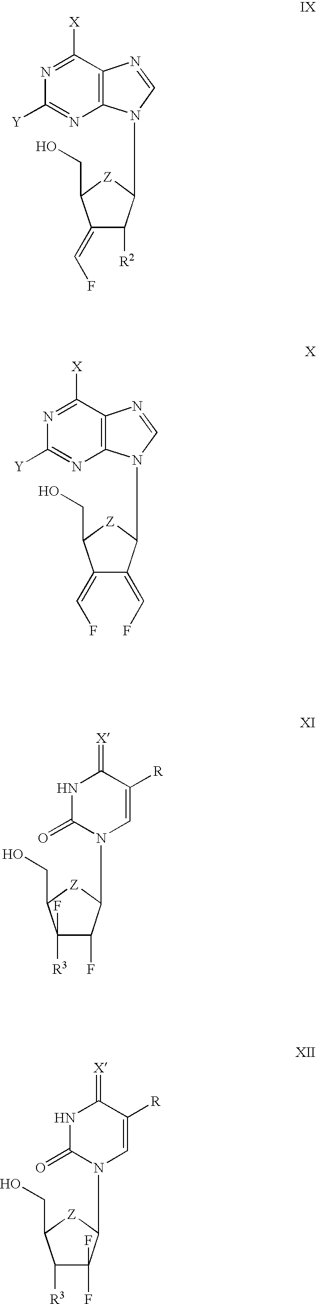

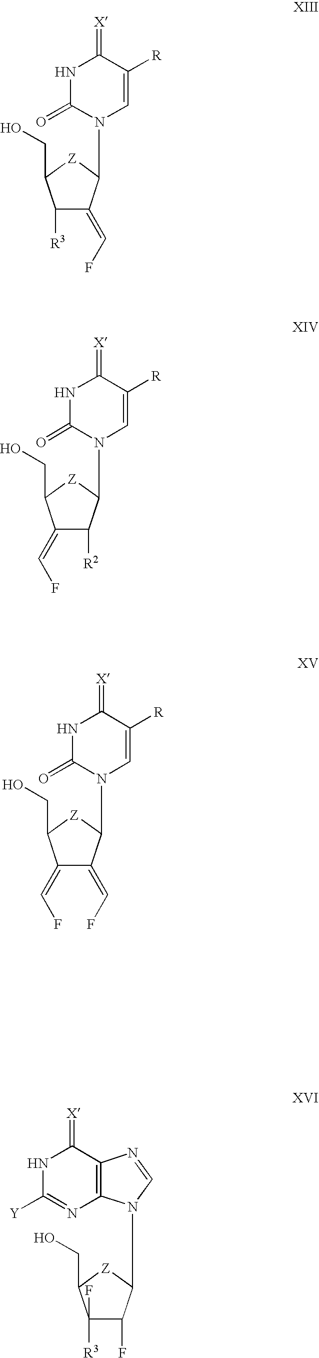

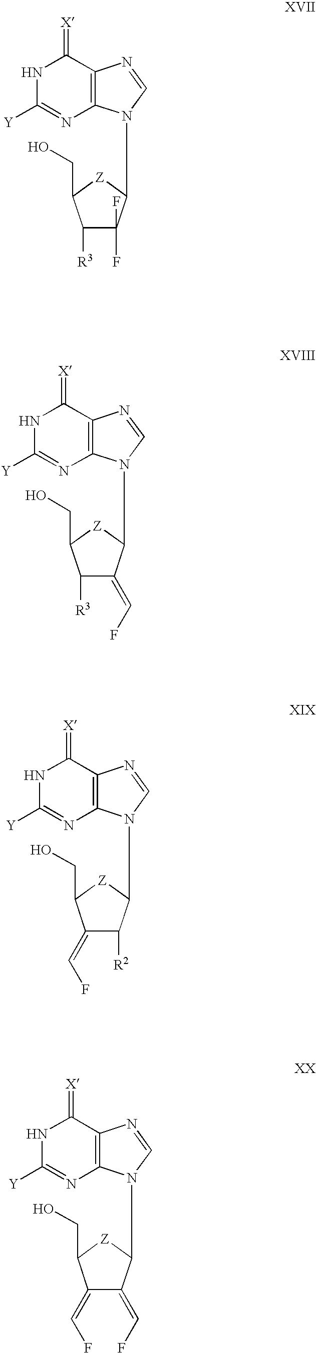

or its pharmaceutically acceptable salt or prodrug thereof, wherein:

(a) R is H, halogen (F, Cl, Br, I), OH, OR′, SH, SR′, NH₂, NHR′, NR′₂, lower alkyl of C₁-C₆, halogenated (F, Cl, Br, I) lower alkyl of C₁-C₆ such as CF₃ and CH₂CH₂F, lower alkenyl of C₂-C₆ such as CH═CH₂, halogenated (F, Cl, Br, I) lower alkenyl of C₂-C₆ such as CH═CHCl, CH═CHBr and CH═CHI, lower alkynyl of C₂-C₆ such as C≡CH, halogenated (F, Cl, Br, I) lower alkynyl of C₂-C₆, lower alkoxy of C₁-C₆ such as CH₂OH and CH₂CH₂OH, CO₂H, CO₂R′, CONH₂, CONHR′, CONR′₂, CH═CHCO₂H, CH═CHCO₂R′;

(b) X and Y are independently H, halogen, OH, OR′, OCH₃, SH, SR′, SCH₃, NH₂, NHR′, NR′₂, CH₃;

(c) each R′ is independently a hydrogen, acyl, lower alkyl of C₁-C₆ or lower cycloalkyl of C₁-C₆;

(d) Z is O, S or CH₂;

(e) R² is F or OH;

(f) R³ is F or OH; and

(g) X′ is O, S, NH, NR′, CH₂, or CHR′;

(h) with the proviso for compound II that when X is NH₂ or compound XII when X is NH and R is H, then R³ is not OH.

In one embodiment of the present invention, a β-D nucleoside of the formula:

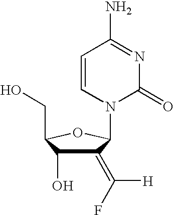

or its pharmaceutically acceptable salt or prodrug thereof, is provided for the treatment or prophylaxis of a Flaviviridae infection, and in particular HCV.

In yet another particular embodiment of the present invention, a β-L nucleoside of the formula:

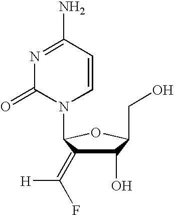

or its pharmaceutically acceptable salt or prodrug thereof, is provided for the treatment or prophylaxis of a disease associated with abnormal cellular proliferation, and in particular a malignant tumor.

In one embodiment of the invention, the nucleoside of the invention is the isolated β-D or β-L isomer. In another embodiment of the invention, the nucleosides are enantiomerically enriched. In yet another embodiment of the invention, the nucleosides is in a enantiomeric mixture in which the desired enantiomer is at least 95%, 98% or 99% pure or free of its corresponding enantiomer.

In another embodiment, the nucleoside has an EC₅₀ (effective concentration to achieve 50% inhibition) when tested in an appropriate cell-based assay, of less than 15 micromolar, and more particularly, less than 10 or 5 micromolar.

Specifically, the invention also includes methods for treating or preventing Flaviviridae infection, including all members of the Hepacivirus genus (HCV), Pestivirus genus (BVDV, CSFV, BDV), or Flavivirus genus (Dengue virus, Japanese encephalitis virus group (including West Nile Virus), and Yellow Fever virus); and abnormal cellular proliferation, including malignant tumors.

The present invention also includes at least the following features:

(a) β-D and β-L nucleosides of the general formula (I)-(XX), or their pharmaceutically acceptable salts or prodrugs thereof, as described herein;

(b) processes for the preparation of the β-D and β-L nucleosides of the general formula (I)-(XX), or their pharmaceutically acceptable salts or prodrugs thereof, as described herein;

(c) pharmaceutical compositions comprising a β-D or β-L nucleoside of the general formula (I)-(XX), or its pharmaceutically acceptable salt or prodrug thereof, in a pharmaceutically acceptable carrier or diluent thereof, as described herein, for the treatment or prophylaxis of a Flaviviridae infection in a host;

(d) pharmaceutical compositions comprising a β-D or β-L nucleoside of the general formula (I)-(XX), or its pharmaceutically acceptable salt or prodrug thereof, in combination with one or more other effective antiviral agent(s), optionally in a pharmaceutically acceptable carrier or diluent thereof, as described herein, for the treatment or prophylaxis of a Flaviviridae infection in a host;

(e) methods for the treatment or prophylaxis of a Flaviviridae infection in a host comprising administering an effective amount of a β-D or β-L nucleoside of the general formula (I)-(XX), or its pharmaceutically acceptable salt or prodrug thereof, optionally in a pharmaceutically acceptable carrier or diluent thereof, as described herein;

(f) methods for the treatment or prophylaxis of a Flaviviridae infection in a host comprising administering an effective amount of a β-D or β-L nucleoside of the general formula (I)-(XX), or its pharmaceutically acceptable salt or prodrug thereof, in combination or alternation with one or more other effective antiviral agent(s), optionally in a pharmaceutically acceptable carrier or diluent thereof, as described herein;

(g) use of a β-D or β-L nucleoside of the general formula (I)-(XX), or its pharmaceutically acceptable salt or prodrug thereof, optionally in a pharmaceutically acceptable carrier, as described herein, for the treatment or prophylaxis of a Flaviviridae infection in a host;

(h) use of a β-D or β-L nucleoside of the general formula (I)-(XX), or its pharmaceutically acceptable salt or prodrug thereof, in combination or alternation with one or more other effective antiviral agent(s), optionally in a pharmaceutically acceptable carrier, as described herein, for the treatment or prophylaxis of a Flaviviridae infection in a host;

(i) use of a β-D or β-L nucleoside of the general formula (I)-(XX), or its pharmaceutically acceptable salt or prodrug thereof, optionally in a pharmaceutically acceptable carrier, as described herein, in the manufacture of a medicament for the treatment or prophylaxis of a Flaviviridae infection in a host;

(j) use of a β-D or β-L nucleoside of the general formula (I)-(XX), or its pharmaceutically acceptable salt or prodrug thereof, in combination or alternation with one or more other effective antiviral agent(s), optionally in a pharmaceutically acceptable carrier, as described herein, in the manufacture of a medicament for the treatment or prophylaxis of a Flaviviridae infection in a host;

(k) use of a β-D or β-L nucleoside of the general formula (I)-(XX), as described herein, or its pharmaceutically acceptable salt or prodrug thereof, optionally in a pharmaceutically acceptable carrier or diluent, as described herein, in a medical therapy, i.e. as antiviral or antitumor/anticancer agent, for example for the treatment or prophylaxis of a Flaviviridae infections, including hepatitis C infection or abnormal cellular proliferation, including a malignant tumor, in a host;

(l) use of a β-D or β-L nucleoside of the general formula (I)-(XX), as described herein, or its pharmaceutically acceptable salt or prodrug thereof, i.e. as antiviral or antitumor/anticancer agent, in combination or alternation with one or more other effective therapeutic agent(s), i.e. another antiviral or antitumor/anticancer agent, optionally in a pharmaceutically acceptable carrier or diluent, as described herein, in a medical therapy, for example for the treatment or prophylaxis of a Flaviviridae infections, including hepatitis C infection or abnormal cellular proliferation, including a malignant tumor, in a host;

(m) pharmaceutical compositions comprising a β-D or β-L nucleoside of the general formula (I)-(XX), or its pharmaceutically acceptable salt or prodrug thereof, in a pharmaceutically acceptable carrier or diluent thereof, as described herein, for the treatment or prophylaxis of a disease associated with abnormal cellular proliferation in a host;

(n) pharmaceutical compositions comprising a β-D or β-L nucleoside of the general formula (I)-(XX), or its pharmaceutically acceptable salt or prodrug thereof, in combination with one or more other effective antiviral agent(s), optionally in a pharmaceutically acceptable carrier or diluent thereof, as described herein, for the treatment or prophylaxis of a disease associated with abnormal cellular proliferation in a host;

(o) methods for the treatment or prophylaxis of a disease associated with abnormal cellular proliferation in a host comprising administering an effective amount of a β-D or β-L nucleoside of the general formula (I)-(XX), or its pharmaceutically acceptable salt or prodrug thereof, optionally in a pharmaceutically acceptable carrier or diluent thereof, as described herein;

(p) methods for the treatment or prophylaxis of a disease associated with abnormal cellular proliferation in a host comprising administering an effective amount of a β-D or β-L nucleoside of the general formula (I)-(XX), or its pharmaceutically acceptable salt or prodrug thereof, in combination or alternation with one or more other effective antiviral agent(s), optionally in a pharmaceutically acceptable carrier or diluent thereof, as described herein;

(q) use of a β-D or β-L nucleoside of the general formula (I)-(XX), or its pharmaceutically acceptable salt or prodrug thereof, optionally in a pharmaceutically acceptable carrier, as described herein, for the treatment or prophylaxis of a disease associated with abnormal cellular proliferation in a host;

(r) use of a β-D or β-L nucleoside of the general formula (I)-(XX), or its pharmaceutically acceptable salt or prodrug thereof, in combination or alternation with one or more other effective antiviral agent(s), optionally in a pharmaceutically acceptable carrier, as described herein, for the treatment or prophylaxis of a disease associated with abnormal cellular proliferation in a host;

(s) use of a β-D or β-L nucleoside of the general formula (I)-(XX), or its pharmaceutically acceptable salt or prodrug thereof, optionally in a pharmaceutically acceptable carrier, as described herein, in the manufacture of a medicament for the treatment or prophylaxis of a disease associated with abnormal cellular proliferation in a host; and

(t) use of a β-D or β-L nucleoside of the general formula (I)-(XX), or its pharmaceutically acceptable salt or prodrug thereof, in combination or alternation with one or more other effective antiviral agent(s), optionally in a pharmaceutically acceptable carrier, as described herein, in the manufacture of a medicament for the treatment or prophylaxis of a disease associated with abnormal cellular proliferation in a host.

### BRIEF DESCRIPTION OF THE FIGURES

FIG. 1 is a graphical depiction of the dose-dependant reduction of the replicon HCV RNA based on treatment with Gemcitabine (♦: ΔCt for HCV RNA). This viral reduction was compared to the reduction of cellular DNA levels (ribosomal DNA) or cellular RNA levels (ribosomal RNA) to obtain the therapeutic index ΔΔCt values (▴: HCV-rDNA ΔΔCt; X: HCV-rRNA ΔΔCt).

FIG. 2 is a graphical depiction of the dose-dependant reduction of the replicon HCV RNA based on treatment with 2′-deoxy-2′-fluorocytidine (♦: ΔCt for HCV RNA). This viral reduction was compared to the reduction of cellular DNA levels (ribosomal DNA) or cellular RNA levels (ribosomal RNA) to obtain the therapeutic index ΔΔCt values (▴: HCV-rDNA ΔΔCt; X: HCV-rRNA ΔΔCt).

### DETAILED DESCRIPTION OF THE INVENTION

The invention is a β-D or β-L nucleoside of the formula (I)-(XX), or its pharmaceutically acceptable salt or prodrug and the use of such compounds for the treatment of a host infected with a virus belonging to the Flaviviridae family. The invention also includes a method for treating a Flaviviridae infection, including an HCV infection, that includes the administration of an anti-viral effective amount of a β-D or β-L nucleoside of the formula (I)-(XX), or its pharmaceutically acceptable salt or prodrug, optionally in a pharmaceutically acceptable carrier or diluent, optionally in combination or alternation with another effective antiviral agent.

Alternatively, a β-L nucleoside of the formula (I)-(XX), or its pharmaceutically acceptable salt or prodrug thereof, can be used for the treatment of abnormal cellular proliferation. The invention also includes a method for treating abnormal cellular proliferation, including a malignant tumor, that includes the administration of an anti-proliferatively effective amount of a β-L nucleoside of the formula (I)-(XX), or its pharmaceutically acceptable salt or prodrug, optionally in a pharmaceutically acceptable carrier or diluent, optionally in combination or alternation with another effective antiproliferative agent.

Specifically, the invention also includes methods for treating or preventing Flaviviridae infection, including all members of the Hepacivirus genus (HCV), Pestivirus genus (BVDV, CSFV, BDV), or Flavivirus genus (Dengue virus, Japanese encephalitis virus group (including West Nile Virus), and Yellow Fever virus); and abnormal cellular proliferation, including malignant tumors.

In an additional embodiment, a method for the treatment or prophylaxis of a mammal having a virus-associated disorder which comprises administering to the mammal a pharmaceutically effective amount of a β-D or β-L nucleoside of the general formula (I)-(XX), or its pharmaceutically acceptable salt or prodrug optionally in a combination or alternation with one or more other anti-viral effective agent(s), optionally in a pharmaceutically acceptable carrier or diluent, as disclosed herein, is provided. In a preferred embodiment, the mammal is a human.

In another embodiment, the use of a β-D or β-L nucleoside of the general formula (I)-(XX), or its pharmaceutically acceptable salt or prodrug optionally in a combination or alternation with one or more other anti-viral effective agent(s), optionally in a pharmaceutically acceptable carrier or diluent, as disclosed herein, for the treatment or prophylaxis of a mammal having a virus-associated disorder is provided. In a preferred embodiment, the mammal is a human.

In an additional embodiment, a method for the treatment or prophylaxis of a mammal having a disorder associated with abnormal cellular proliferation which comprises administering to the mammal a pharmaceutically effective amount of a β-D or β-L nucleoside of the general formula (III)-(V) or (VIII)-(X), or its pharmaceutically acceptable salt or prodrug optionally in a combination or alternation with one or more other anti-proliferatively effective agent(s), optionally in a pharmaceutically acceptable carrier or diluent, as disclosed herein, is provided. In a preferred embodiment, the mammal is a human.

In another embodiment, the use of a β-D or β-L nucleoside of the general formula (I)-(XX), or its pharmaceutically acceptable salt or prodrug thereof, optionally in a combination or alternation with one or more other anti-proliferatively effective agent(s), optionally in a pharmaceutically acceptable carrier or diluent, as disclosed herein, for the treatment or prophylaxis of a mammal having a disorder associated with abnormal cellular proliferation is provided. In a preferred embodiment, the mammal is a human.

The Flaviviridaeviruses that can be treated include Flaviviruses, including the Dengue virus group (Dengue virus, Dengue virus type 1, Dengue virus type 2, Dengue virus type 3, Dengue virus type 4), the Japanese encephalitis virus group (Alfuy Virus, Japanese encephalitis virus, Kookaburra virus, Koutango virus, Kunjin virus, Murray Valley encephalitis virus, St. Louis encephalitis virus, Stratford virus, Usutu virus, West Nile Virus), the Modoc virus group, the Rio Bravo virus group (Apoi virus, Rio Brovo virus, Saboya virus), the Ntaya virus group, the Tick-Borne encephalitis group (tick born encephalitis virus), the Tyuleniy virus group, Uganda S virus group and the Yellow Fever virus group; Pestiviruses, including Bovine Viral Diarrhea Virus-2 (BVDV-2), Pestivirus type 1 (including BVDV), Pestivirus type 2 (including Hog Cholera Virus) and Pestivirus type 3 (including Border Disease Virus), and Hepaciviruses, including hepatitis C virus (HCV), which is composed of many clades, types and subtypes.

I. Disorders Characterized by Abnormal Cellular Proliferation

Non-limiting examples of proliferative disorders that can be treated and/or imaged with a compound or composition of the present invention include those in Table 1, as well as any others listed or described in the Background of the Invention or otherwise in the specification.

TABLE 1

| Organ System | Disease/Pathology |
|---|---|
| Dermatological | Psoriasis (all forms), acne vulgaris, acne |
| | rosacea, common warts, anogenital (venereal) |
| | warts, eczema; lupus associated skin lesions; |
| | dermatitides such as seborrheic dermatitis and |
| | solar dermatitis; keratoses such as seborrheic |
| | keratosis, senile keratosis, actinic keratosis, |
| | photo-induced keratosis, skin aging, including |
| | photo-induced skin aging, keratosis follicularis, |
| | keloids |
| | and |
| | Prophylaxis against keloid formation; |
| | leukoplakia, lichen, planus, keratitis, contact |
| | dermatitis, eczema, urticaria, pruritus, |
| | hidradenitis, acne inversa |
| Cardiovascular | Hypertension, vasculo-occlusive diseases |
| | including Atherosclerosis, thrombosis and |
| | restenosis after angioplasty; acute coronary |
| | syndromes such as unstable angina, myocardial |
| | infarction, ischemic and non-ischemic |
| | cardiomyopathies, post-MI cardiomyopathy |
| | and myocardial fibrosis, substance-induced |
| | cardiomyopathy. |
| Endocrine | Insulin resistant states including obesity, |
| | diabetes mellitus (types 1 & 2), diabetic |
| | retinopathy, macular degeneration associated |
| | with diabetes, gestational diabetes, impaired |
| | glucose tolerance, polycystic ovarian |
| | syndrome; osteoporosis, osteopenia, accelerated |
| | aging of tissues and organs including Werner's |
| | syndrome. |
| Urogenital | Endometriosis, benign prostatic hyperplasia, |
| | leiomyoma, Polycystic kidney disease, diabetic |
| | nephropathy. |
| Pulmonary | Asthma, chronic obstructive pulmonary disease |
| | (COPD), reactive Airway disease, pulmonary |
| | fibrosis, pulmonary hypertension. |
| Connective tissue/joints | Immunological Rheumatoid arthritis, |
| | Raynaud's phenomenon/disease, Sjogren's |
| | Syndrome, systemic sclerosis, systemic lupus |
| | erythematosus, vasculitides, ankylosing |
| | spondylitis, osteoarthritis, reactive arthritis, |
| | psoriatic arthritis, fibromyalgia. |
| Other | Fibrocystic breast disease, fibroadenoma, |
| | chronic fatigue syndrome. |

Nonlimiting examples of neoplastic diseases or malignancies treatable and/or diagnosable with a compound or composition of the present invention are listed in Table 2.

TABLE 2

| Organ System | Malignancy/Cancer type |
|---|---|
| Skin | Basal cell carcinoma, melanoma, squamous cell |
| | carcinoma; cutaneous T cell lymphoma; Kaposi's |
| | sarcoma. |
| Hematological | Acute leukemia, chronic leukemia and myelodysplastic |
| | syndromes. |
| Urogenital | Prostatic, renal and bladder carcinomas, anogenital |
| | carcinomas including cervical, ovarian, uterine, vulvar, |
| | vaginal, and those associated with human papilloma virus |
| | infection. |
| Neurological | Gliomas including glioblastomas, astrocytoma, |
| | ependymoma, medulloblastoma, oligodendroma; |
| | meningioma, pituitary adenoma, neuroblastoma, |
| | craniopharyngioma. |
| Gastrointestinal | Colon, colorectal, gastric, esophageal, mucocutaneous |
| | carcinomas. |
| Breast | Breast cancer including estrogen receptor and |
| | progesterone Receptor positive or negative subtypes, |
| | soft tissue tumors. |
| Metastasis | Metastases resulting from the neoplasms. |
| Skeletal | Osteogenic sarcoma, malignant fibrou histeocytoma, |
| | chondrosarcoma, rhabdomyosarcoma, leiomyosarcoma, |
| | mycloma. |
| Diffuse Tumors | Lymphoma (non-Hodgkin's or Hodgkin's), sickle cell |
| | anemia. |
| Other | Angiomata, angiogenesis associated with the neoplasms. |

II. Compounds of the Invention

In one embodiment, the nucleoside is a β-D or β-L nucleoside of the general formula (I)-(XX):

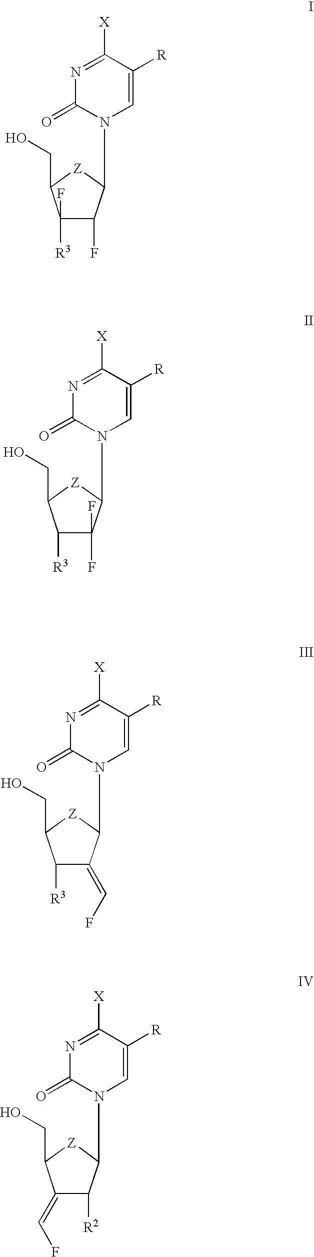

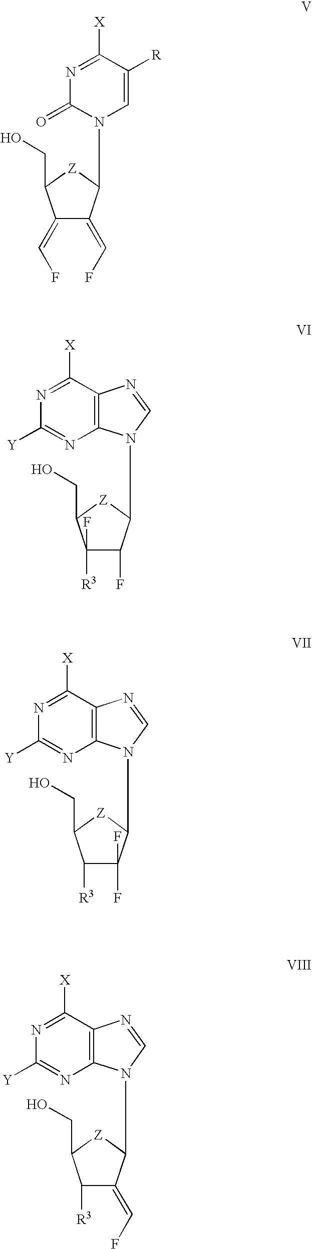

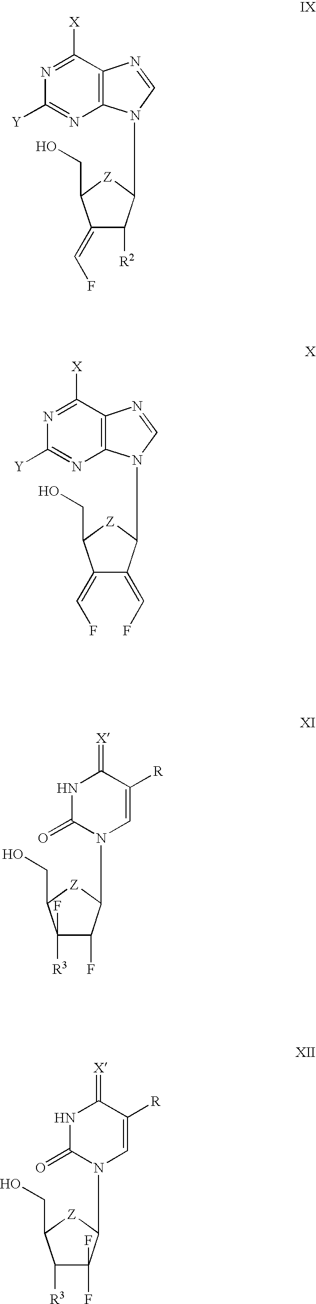

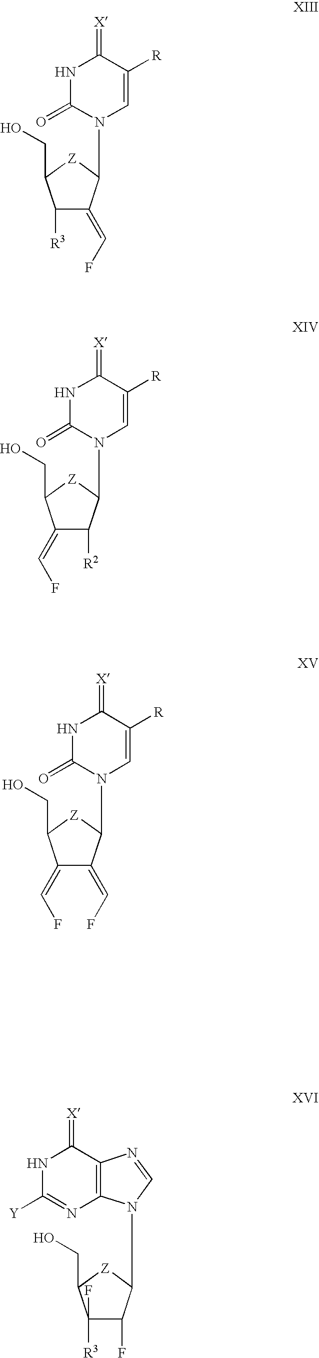

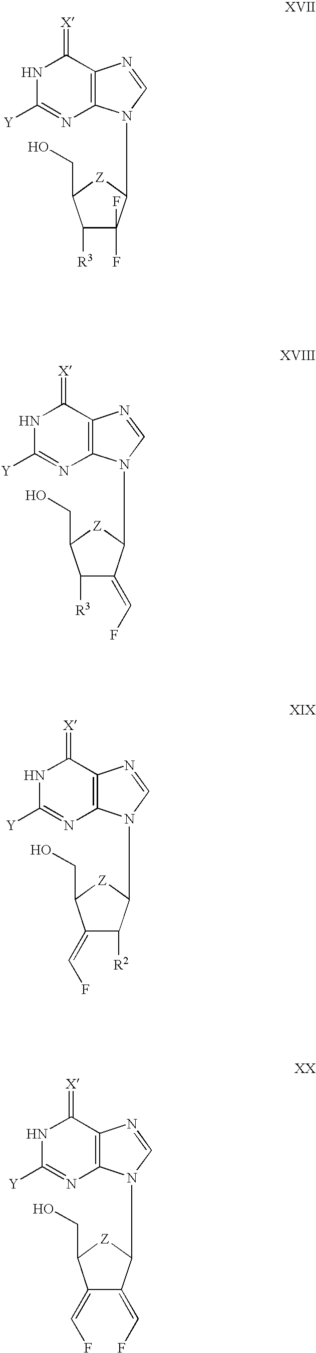

or its pharmaceutically acceptable salt or prodrug thereof, or its use as further described herein wherein:

(a) R is H, halogen (F, Cl, Br, I), OH, OR′, SH, SR′, NH₂, NHR′, NR′₂, lower alkyl of C₁-C₆, halogenated (F, Cl, Br, I) lower alkyl of C₁-C₆ such as CF₃ and CH₂CH₂F, lower alkenyl of C₂-C₆ such as CH═CH₂, halogenated (F, Cl, Br, I) lower alkenyl of C₂-C₆ such as CH═CHCl, CH═CHBr and CH═CHI, lower alkynyl of C₂-C₆ such as C≡CH, halogenated (F, Cl, Br, I) lower alkynyl of C₂-C₆, lower alkoxy of C₁-C₆ such as CH₂OH and CH₂CH₂OH, CO₂H, CO₂R′, CONH₂, CONHR′, CONR′₂, CH═CHCO₂H, CH═CHCO₂R′;

(b) X and Y are independently H, halogen, OH, OR′, OCH₃, SH, SR′, SCH₃, NH₂, NHR′, NR′₂, CH₃;

(c) each R′ is independently a hydrogen, acyl, lower alkyl of C₁-C₆ or lower cycloalkyl of C₁-C₆;

(d) Z is O, S or CH₂;

(e) R² is F or OH;

(f) R³ is F or OH; and

(g) X′ is O, S, NH, NR′, CH₂, or CHR′;

(h) with the proviso for compound II that when X is NH₂ or compound XII when X is NH and Ris H, then R³ is not OH.

In one embodiment, the fluorinated derivatives are preferred.

In another embodiment, the gem-difluoro-nucleosides are preferred.

In an important embodiment, none of the aspects of the invention include gemcitabine (β-D-2′,2′-difuoro-2′ deoxycytidine).

In yet another embodiment, the 2′-(fluoromethylidene) and/or 3′-(fluoromethylidene) nucleosides, the vinylogous analogs of 2′-fluoro-2′-deoxy nucleosides, are preferred. In particular, E configuration is preferred.

The present invention provides a β-D or β-L nucleosides of the formula (I)-(XX), or its pharmaceutically acceptable salt or prodrug and the use of such compounds for the treatment of a host infected with a virus belonging to the Flaviviridae family, as well as β-L nucleoside of the formula (I)-(XX), or its pharmaceutically acceptable salt or prodrug thereof, and the use of such compounds are provided for the treatment of abnormal cellular proliferation.

In yet another particular embodiment of the present invention, a β-D nucleoside of the formula:

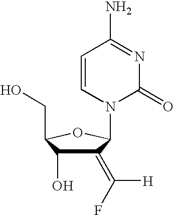

or its pharmaceutically acceptable salt or prodrug thereof, is provided for the treatment or prophylaxis of a Flaviviridae infection, and in particular HCV.

In yet another particular embodiment of the present invention, a β-L nucleoside of the formula:

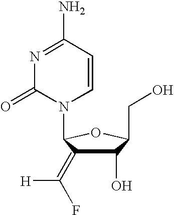

or its pharmaceutically acceptable salt or prodrug thereof, is provided for the treatment or prophylaxis of a disease associated with abnormal cellular proliferation, and in particular a malignant tumor.

In yet another particular embodiment of the present invention, a β-D nucleoside of the formula:

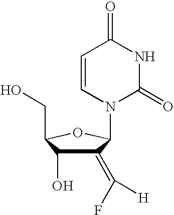

or its pharmaceutically acceptable salt or prodrug thereof, is provided for the treatment or prophylaxis of a Flaviviridae infection, and in particular HCV.

In yet another particular embodiment of the present invention, a β-L nucleoside of the formula:

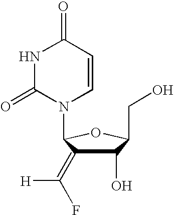

or its pharmaceutically acceptable salt or prodrug thereof, is provided for the treatment or prophylaxis of a disease associated with abnormal cellular proliferation, and in particular a malignant tumor.

In yet another embodiment, the nucleoside is a β-D or β-L nucleoside of the general formula (I)-(XX):

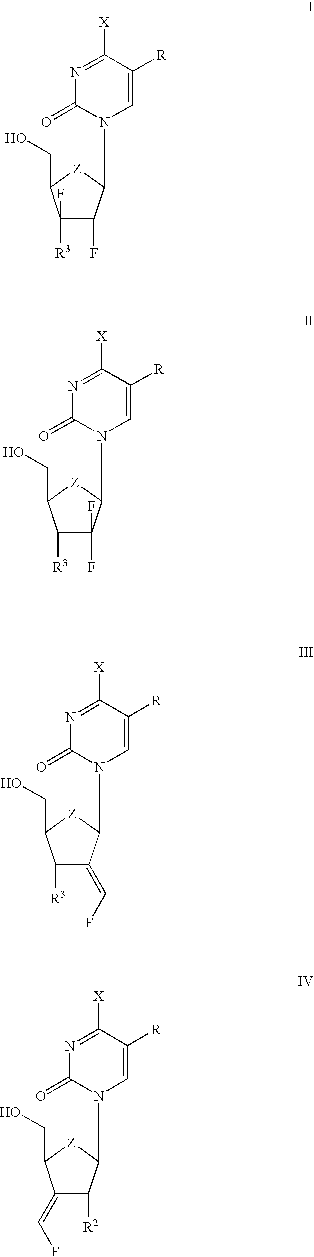

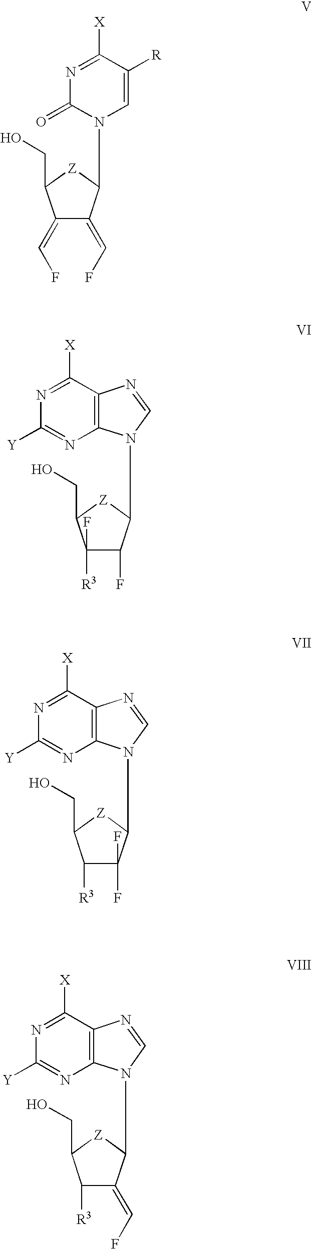

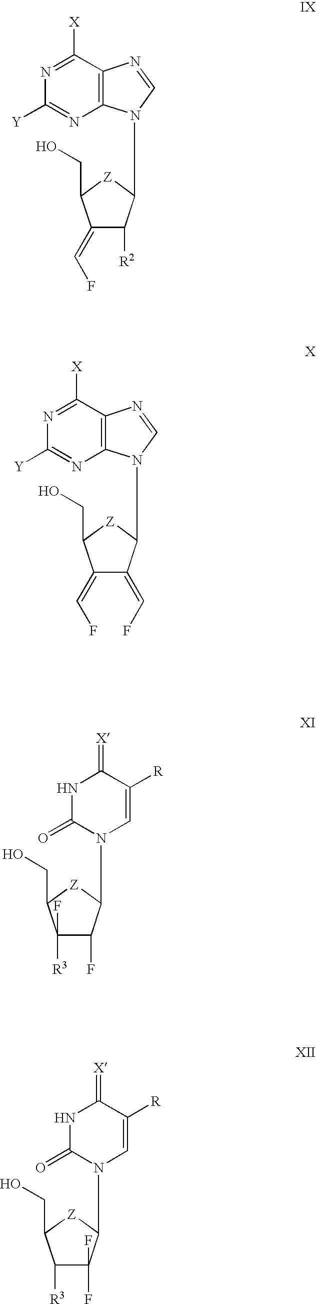

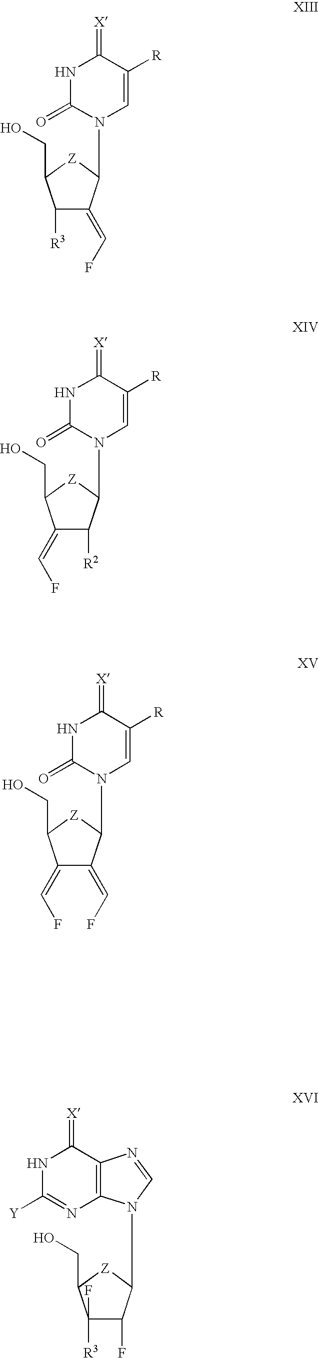

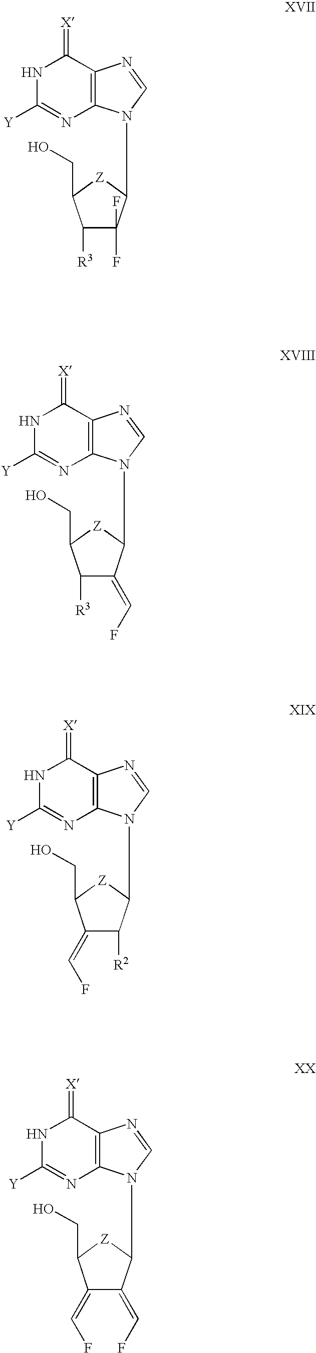

or its pharmaceutically acceptable salt or prodrug thereof or its use as further described herein, wherein R is H.

In another embodiment, the nucleoside is a β-D or β-L nucleoside of the general formula (I)-(XX) or its pharmaceutically acceptable salt or prodrug thereof or its use as further described herein, wherein R is halogen (F, Cl, Br, I).

In another embodiment, the nucleoside is a β-D or β-L nucleoside of the general formula (I)-(XX) or its pharmaceutically acceptable salt or prodrug thereof or its use as further described herein, wherein R is OH.

In another embodiment, the nucleoside is a β-D or β-L nucleoside of the general formula (I)-(XX) or its pharmaceutically acceptable salt or prodrug thereof or its use as further described herein, wherein R is OR′.

In another embodiment, the nucleoside is a β-D or β-L nucleoside of the general formula (I)-(XX) or its pharmaceutically acceptable salt or prodrug thereof or its use as further described herein, wherein R is SH.

In another embodiment, the nucleoside is a β-D or β-L nucleoside of the general formula (I)-(XX) or its pharmaceutically acceptable salt or prodrug thereof or its use as further described herein, wherein R is SR′.

In another embodiment, the nucleoside is a β-D or β-L nucleoside of the general formula (I)-(XX) or its pharmaceutically acceptable salt or prodrug thereof or its use as further described herein, wherein R is NH₂.

In another embodiment, the nucleoside is a β-D or β-L nucleoside of the general formula (I)-(XX) or its pharmaceutically acceptable salt or prodrug thereof or its use as further described herein, wherein R is NHR′.

In another embodiment, the nucleoside is a β-D or β-L nucleoside of the general formula (I)-(XX) or its pharmaceutically acceptable salt or prodrug thereof or its use as further described herein, wherein R is NR′₂.

In another embodiment, the nucleoside is a β-D or β-L nucleoside of the general formula (I)-(XX) or its pharmaceutically acceptable salt or prodrug thereof or its use as further described herein, wherein R is lower alkyl of C₁-C₆.

In another embodiment, the nucleoside is a β-D or β-L nucleoside of the general formula (I)-(XX) or its pharmaceutically acceptable salt or prodrug thereof or its use as further described herein, wherein R is halogenated (F, Cl, Br, I) lower alkyl of C₁-C₆ including CF₃ and CH₂CH₂F.

In another embodiment, the nucleoside is a β-D or β-L nucleoside of the general formula (I)-(XX) or its pharmaceutically acceptable salt or prodrug thereof or its use as further described herein, wherein R is lower alkenyl of C₂-C₆ including CH═CHCl, CH═CHBr and CH═CHI.

In another embodiment, the nucleoside is a β-D or β-L nucleoside of the general formula (I)-(XX) or its pharmaceutically acceptable salt or prodrug thereof or its use as further described herein, wherein R is lower alkynyl of C₂-C₆ including C≡CH.

In another embodiment, the nucleoside is a β-D or β-L nucleoside of the general formula (I)-(XX) or its pharmaceutically acceptable salt or prodrug thereof or its use as further described herein, wherein R is halogenated (F, Cl, Br, I) lower alkynyl of C₂-C₆.

In another embodiment, the nucleoside is a β-D or β-L nucleoside of the general formula (I)-(XX) or its pharmaceutically acceptable salt or prodrug thereof or its use as further described herein, wherein R is lower alkoxy of C₁-C₆ including CH₂OH and CH₂CH₂OH.

In another embodiment, the nucleoside is a β-D or β-L nucleoside of the general formula (I)-(XX) or its pharmaceutically acceptable salt or prodrug thereof or its use as further described herein, wherein R is CO₂H.

In another embodiment, the nucleoside is a β-D or β-L nucleoside of the general formula (I)-(XX) or its pharmaceutically acceptable salt or prodrug thereof or its use as further described herein, wherein R is CO₂R′.

In another embodiment, the nucleoside is a β-D or β-L nucleoside of the general formula (I)-(XX) or its pharmaceutically acceptable salt or prodrug thereof or its use as further described herein, wherein R is CONH₂.

In another embodiment, the nucleoside is a β-D or β-L nucleoside of the general formula (I)-(XX) or its pharmaceutically acceptable salt or prodrug thereof or its use as further described herein, wherein R is CONHR′.

In another embodiment, the nucleoside is a β-D or β-L nucleoside of the general formula (I)-(XX) or its pharmaceutically acceptable salt or prodrug thereof or its use as further described herein, wherein R is CONR′₂.

In another embodiment, the nucleoside is a β-D or β-L nucleoside of the general formula (I)-(XX) or its pharmaceutically acceptable salt or prodrug thereof or its use as further described herein, wherein R is CH═CHCO₂H.

In another embodiment, the nucleoside is a β-D or β-L nucleoside of the general formula (I)-(XX) or its pharmaceutically acceptable salt or prodrug thereof or its use as further described herein, wherein R is CH═CHCO₂R′.

In another embodiment, the nucleoside is a β-D or β-L nucleoside of the general formula (I)-(XX) or its pharmaceutically acceptable salt or prodrug thereof or its use as further described herein, wherein X and Y are H.

In another embodiment, the nucleoside is a β-D or β-L nucleoside of the general formula (I)-(XX) or its pharmaceutically acceptable salt or prodrug thereof or its use as further described herein, wherein X and Y are halogen.

In another embodiment, the nucleoside is a β-D or β-L nucleoside of the general formula (I)-(XX) or its pharmaceutically acceptable salt or prodrug thereof or its use as further described herein, wherein X and Y are OR′.

In another embodiment, the nucleoside is a β-D or β-L nucleoside of the general formula (I)-(XX) or its pharmaceutically acceptable salt or prodrug thereof or its use as further described herein, wherein X and Y are OCH₃.

In another embodiment, the nucleoside is a β-D or β-L nucleoside of the general formula (I)-(XX) or its pharmaceutically acceptable salt or prodrug thereof or its use as further described herein, wherein X and Y are SH.

In another embodiment, the nucleoside is a β-D or β-L nucleoside of the general formula (I)-(XX) or its pharmaceutically acceptable salt or prodrug thereof or its use as further described herein, wherein X and Y are SR′.

In another embodiment, the nucleoside is a β-D or β-L nucleoside of the general formula (I)-(XX) or its pharmaceutically acceptable salt or prodrug thereof or its use as further described herein, wherein X and Y are SCH₃.

In another embodiment, the nucleoside is a β-D or β-L nucleoside of the general formula (I)-(XX) or its pharmaceutically acceptable salt or prodrug thereof or its use as further described herein, wherein X and Y are NH₂.

In another embodiment, the nucleoside is a β-D or β-L nucleoside of the general formula (I)-(XX) or its pharmaceutically acceptable salt or prodrug thereof or its use as further described herein, wherein X and Y are NHR′.

In another embodiment, the nucleoside is a β-D or β-L nucleoside of the general formula (I)-(XX) or its pharmaceutically acceptable salt or prodrug thereof or its use as further described herein, wherein X and Y are NR′₂.

In another embodiment, the nucleoside is a β-D or β-L nucleoside of the general formula (I)-(XX) or its pharmaceutically acceptable salt or prodrug thereof or its use as further described herein, wherein X and Y are CH₃.

In another embodiment, the nucleoside is a β-D or β-L nucleoside of the general formula (I)-(XX) or its pharmaceutically acceptable salt or prodrug thereof or its use as further described herein, wherein each R′ is independently is hydrogen.

In another embodiment, the nucleoside is a β-D or β-L nucleoside of the general formula (I)-(XX) or its pharmaceutically acceptable salt or prodrug thereof or its use as further described herein, wherein each R′ is independently lower alkyl of C₁-C₆.

In another embodiment, the nucleoside is a β-D or β-L nucleoside of the general formula (I)-(XX) or its pharmaceutically acceptable salt or prodrug thereof or its use as further described herein, wherein each R′ is independently lower cycloalkyl of C₁-C₆.

In another embodiment, the nucleoside is a β-D or β-L nucleoside of the general formula (I)-(XX) or its pharmaceutically acceptable salt or prodrug thereof or its use as further described herein, wherein Z is O.

In another embodiment, the nucleoside is a β-D or β-L nucleoside of the general formula (I)-(XX) or its pharmaceutically acceptable salt or prodrug thereof or its use as further described herein, wherein Z is S.

In another embodiment, the nucleoside is a β-D or β-L nucleoside of the general formula (I)-(XX) or its pharmaceutically acceptable salt or prodrug thereof or its use as further described herein, wherein Z is CH₂.

In another embodiment, the nucleoside is a β-D or β-L nucleoside of the general formula (I)-(XX) or its pharmaceutically acceptable salt or prodrug thereof or its use as further described herein, wherein R² is F.

In another embodiment, the nucleoside is a β-D or β-L nucleoside of the general formula (I)-(XX) or its pharmaceutically acceptable salt or prodrug thereof or its use as further described herein, wherein R² is OH

In another embodiment, the nucleoside is a β-D or β-L nucleoside of the general formula (I)-(XX) or its pharmaceutically acceptable salt or prodrug thereof or its use as further described herein, wherein R³ is F.

In another embodiment, the nucleoside is a β-D or β-L nucleoside of the general formula (I)-(XX) or its pharmaceutically acceptable salt or prodrug thereof or its use as further described herein, wherein R³ is OH.

In another embodiment, the nucleoside is a β-D or β-L nucleoside of the general formula (I)-(XX) or its pharmaceutically acceptable salt or prodrug thereof or its use as further described herein, wherein X′ is O.

In another embodiment, the nucleoside is a β-D or β-L nucleoside of the general formula (I)-(XX) or its pharmaceutically acceptable salt or prodrug thereof or its use as further described herein, wherein X′ is S.

In another embodiment, the nucleoside is a β-D or β-L nucleoside of the general formula (I)-(XX) or its pharmaceutically acceptable salt or prodrug thereof or its use as further described herein, wherein X′ is NH.

In another embodiment, the nucleoside is a β-D or β-L nucleoside of the general formula (I)-(XX) or its pharmaceutically acceptable salt or prodrug thereof or its use as further described herein, wherein X′ is NR′.

In another embodiment, the nucleoside is a β-D or β-L nucleoside of the general formula (I)-(XX) or its pharmaceutically acceptable salt or prodrug thereof or its use as further described herein, wherein X′ is CH₂.

In another embodiment, the nucleoside is a β-D or β-L nucleoside of the general formula (I)-(XX) or its pharmaceutically acceptable salt or prodrug thereof or its use as further described herein, wherein X′ is CHR′.

In another embodiment, the nucleoside is a β-D or β-L nucleoside of the general formula (I)-(XX) or its pharmaceutically acceptable salt or prodrug thereof or its use as further described herein, wherein: R is halogen; X and Y are NH₂.

In another embodiment, the nucleoside is a β-D or β-L nucleoside of the general formula (I)-(XX) or its pharmaceutically acceptable salt or prodrug thereof or its use as further described herein, wherein: R is halogen; Z is O; and R³ is OH.

In another embodiment, the nucleoside is a β-D or β-L nucleoside of the general formula (I)-(XX) or its pharmaceutically acceptable salt or prodrug thereof or its use as further described herein, wherein: R is alkyl; Z is O; and R³ is OH.

In another embodiment, the nucleoside is a β-D or β-L nucleoside of the general formula (I)-(XX) or its pharmaceutically acceptable salt or prodrug thereof or its use as further described herein, wherein: R is H; Z is O; R³′ is OH and R³is F.

In another embodiment, the nucleoside is a β-D or β-L nucleoside of the general formula (I)-(XX) or its pharmaceutically acceptable salt or prodrug thereof or its use as further described herein, wherein: R is alkyl; X and Y are NH₂; R³ is OH.

In another embodiment, the nucleoside is a β-D or β-L nucleoside of the general formula (I)-(XX) or its pharmaceutically acceptable salt or prodrug thereof or its use as further described herein, wherein R is halogen; R³ is OH; Z is O; and R³′ is F.

In another embodiment of the present invention, a β-D or β-L nucleoside of the formula:

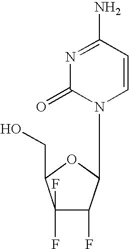

or its pharmaceutically acceptable salt or prodrug thereof or its use as further described herein is provided.

In another embodiment of the present invention, a β-D or β-L nucleoside of the formula:

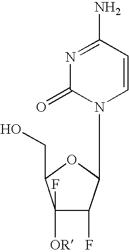

or its pharmaceutically acceptable salt or prodrug thereof or its use as further described herein is provided.

In another embodiment of the present invention, a β-D or β-L nucleoside of the formula:

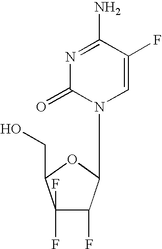

or its pharmaceutically acceptable salt or prodrug thereof or its use as further described herein is provided.

In another embodiment of the present invention, a β-D or β-L nucleoside of the formula:

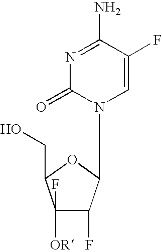

or its pharmaceutically acceptable salt or prodrug thereof or its use as further described herein is provided.

In another embodiment of the present invention, a β-D or β-L nucleoside of the formula:

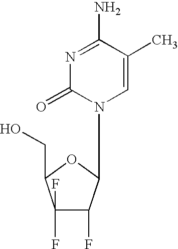

or its pharmaceutically acceptable salt or prodrug thereof or its use as further described herein is provided.

In another embodiment of the present invention, a β-D or β-L nucleoside of the formula:

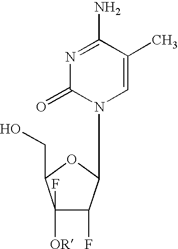

or its pharmaceutically acceptable salt or prodrug thereof or its use as further described herein is provided.

In another embodiment of the present invention, a β-D or β-L nucleoside of the formula:

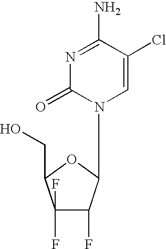

or its pharmaceutically acceptable salt or prodrug thereof or its use as further described herein is provided.

In another embodiment of the present invention, a β-D or β-L nucleoside of the formula:

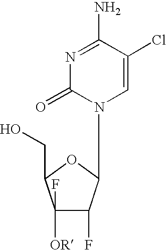

or its pharmaceutically acceptable salt or prodrug thereof or its use as further described herein is provided.

In another embodiment of the present invention, a β-D or β-L nucleoside of the formula:

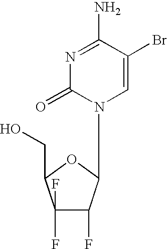

or its pharmaceutically acceptable salt or prodrug thereof or its use as further described herein is provided.

In another embodiment of the present invention, a β-D or β-L nucleoside of the formula:

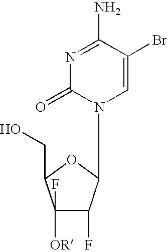

or its pharmaceutically acceptable salt or prodrug thereof or its use as further described herein is provided.

In another embodiment of the present invention, a β-D or β-L nucleoside of the formula:

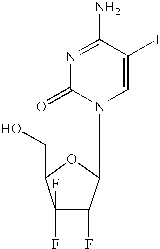

or its pharmaceutically acceptable salt or prodrug thereof or its use as further described herein is provided.

In another embodiment of the present invention, a β-D or β-L nucleoside of the formula:

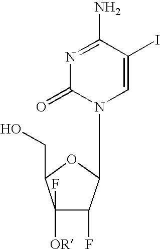

or its pharmaceutically acceptable salt or prodrug thereof or its use as further described herein is provided.

In another embodiment of the present invention, a β-D or β-L nucleoside of the formula:

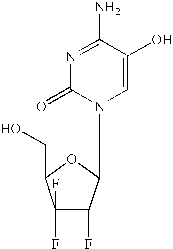

or its pharmaceutically acceptable salt or prodrug thereof or its use as further described herein is provided.

In another embodiment of the present invention, a β-D or β-L nucleoside of the formula:

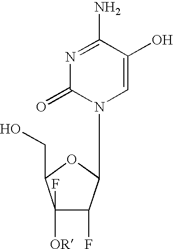

or its pharmaceutically acceptable salt or prodrug thereof or its use as further described herein is provided.

In another embodiment of the present invention, a β-D or β-L nucleoside of the formula:

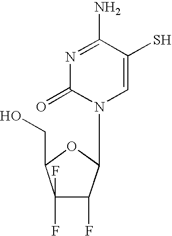

or its pharmaceutically acceptable salt or prodrug thereof or its use as further described herein is provided.

In another embodiment of the present invention, a β-D or β-L nucleoside of the formula:

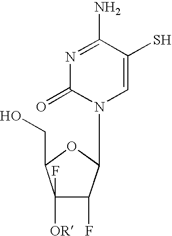

or its pharmaceutically acceptable salt or prodrug thereof or its use as further described herein is provided.

In another embodiment of the present invention, a β-D or β-L nucleoside of the formula:

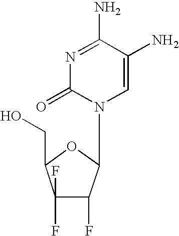

or its pharmaceutically acceptable salt or prodrug thereof or its use as further described herein is provided.

In another embodiment of the present invention, a β-D or β-L nucleoside of the formula:

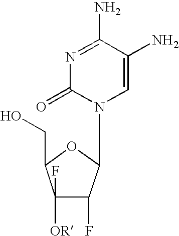

or its pharmaceutically acceptable salt or prodrug thereof or its use as further described herein is provided.

In another embodiment of the present invention, a β-D or β-L nucleoside of the formula:

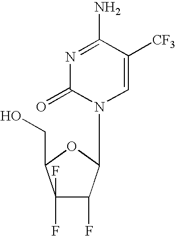

or its pharmaceutically acceptable salt or prodrug thereof or its use as further described herein is provided.

In another embodiment of the present invention, a β-D or β-L nucleoside of the formula:

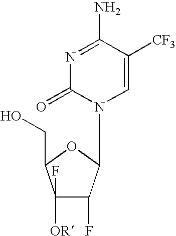

or its pharmaceutically acceptable salt or prodrug thereof or its use as further described herein is provided.

In another embodiment of the present invention, a β-D or β-L nucleoside of the formula:

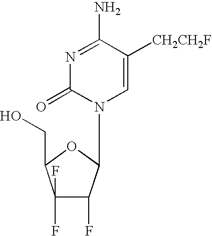

or its pharmaceutically acceptable salt or prodrug thereof or its use as further described herein is provided.

In another embodiment of the present invention, a β-D or β-L nucleoside of the formula:

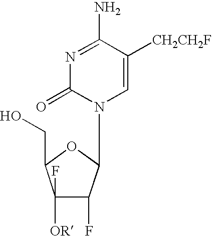

or its pharmaceutically acceptable salt or prodrug thereof or its use as further described herein is provided.

In another embodiment of the present invention, a β-D or β-L nucleoside of the formula:

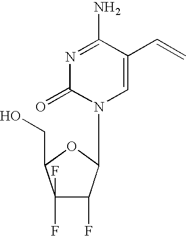

or its pharmaceutically acceptable salt or prodrug thereof or its use as further described herein is provided.

In another embodiment of the present invention, a β-D or β-L nucleoside of the formula:

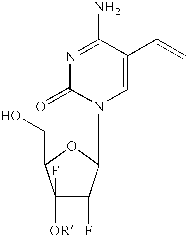

or its pharmaceutically acceptable salt or prodrug thereof or its use as further described herein is provided.

In another embodiment of the present invention, a β-D or β-L nucleoside of the formula:

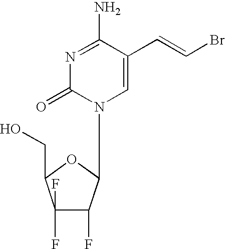

or its pharmaceutically acceptable salt or prodrug thereof or its use as further described herein is provided.

In another embodiment of the present invention, a β-D or β-L nucleoside of the formula:

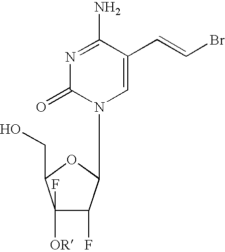

or its pharmaceutically acceptable salt or prodrug thereof or its use as further described herein is provided.

In another embodiment of the present invention, a β-D or β-L nucleoside of the formula:

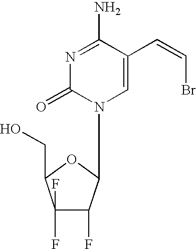

or its pharmaceutically acceptable salt or prodrug thereof or its use as further described herein is provided.

In another embodiment of the present invention, a β-D or β-L nucleoside of the formula:

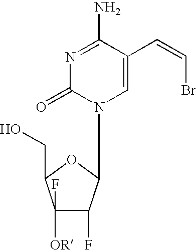

or its pharmaceutically acceptable salt or prodrug thereof or its use as further described herein is provided.

In another embodiment of the present invention, a β-D or β-L nucleoside of the formula:

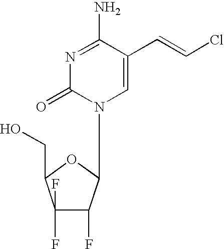

or its pharmaceutically acceptable salt or prodrug thereof or its use as further described herein is provided.

In another embodiment of the present invention, a β-D or β-L nucleoside of the formula:

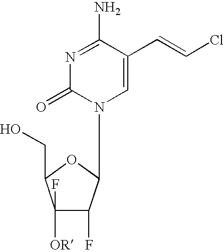

or its pharmaceutically acceptable salt or prodrug thereof or its use as further described herein is provided.

In another embodiment of the present invention, a β-D or β-L nucleoside of the formula:

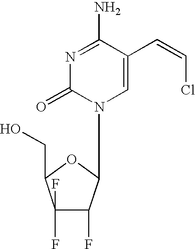

or its pharmaceutically acceptable salt or prodrug thereof or its use as further described herein is provided.

In another embodiment of the present invention, a β-D or β-L nucleoside of the formula:

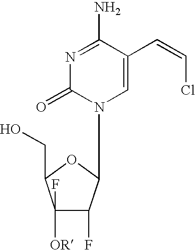

or its pharmaceutically acceptable salt or prodrug thereof or its use as further described herein is provided.

In another embodiment of the present invention, a β-D or β-L nucleoside of the formula:

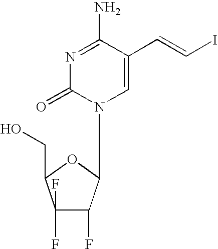

or its pharmaceutically acceptable salt or prodrug thereof or its use as further described herein is provided.

In another embodiment of the present invention, a β-D or 62 -L nucleoside of the formula:

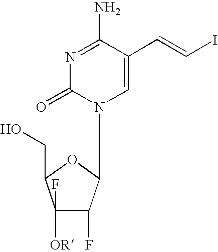

or its pharmaceutically acceptable salt or prodrug thereof or its use as further described herein is provided.

In another embodiment of the present invention, a β-D or β-L nucleoside of the formula:

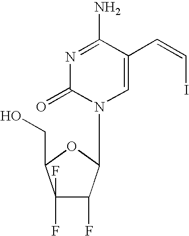

or its pharmaceutically acceptable salt or prodrug thereof or its use as further described herein is provided.

In another embodiment of the present invention, a β-D or β-L nucleoside of the formula:

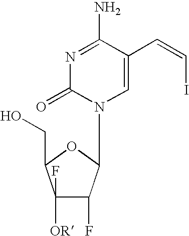

or its pharmaceutically acceptable salt or prodrug thereof or its use as further described herein is provided.

In another embodiment of the present invention, a β-D or β-L nucleoside of the formula:

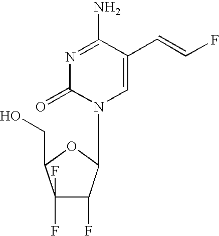

or its pharmaceutically acceptable salt or prodrug thereof or its use as further described herein is provided.

In another embodiment of the present invention, a β-D or β-L nucleoside of the formula:

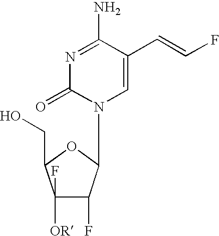

or its pharmaceutically acceptable salt or prodrug thereof or its use as further described herein is provided.

In another embodiment of the present invention, a β-D or β-L nucleoside of the formula:

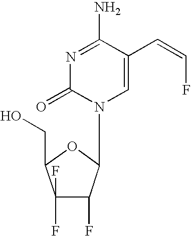

or its pharmaceutically acceptable salt or prodrug thereof or its use as further described herein is provided.

In another embodiment of the present invention, a β-D or β-L nucleoside of the formula:

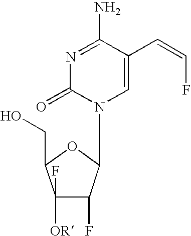

or its pharmaceutically acceptable salt or prodrug thereof or its use as further described herein is provided.

In another embodiment of the present invention, a β-D or β-L nucleoside of the formula:

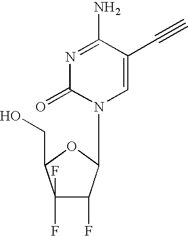

or its pharmaceutically acceptable salt or prodrug thereof or its use as further described herein is provided.

In another embodiment of the present invention, a β-D or β-L nucleoside of the formula:

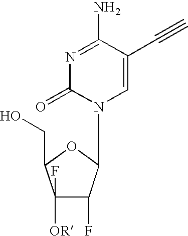

or its pharmaceutically acceptable salt or prodrug thereof or its use as further described herein is provided.

In another embodiment of the present invention, a β-D or β-L nucleoside of the formula:

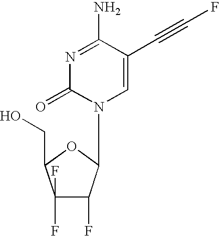

or its pharmaceutically acceptable salt or prodrug thereof or its use as further described herein is provided.

In another embodiment of the present invention, a β-D or β-L nucleoside of the formula:

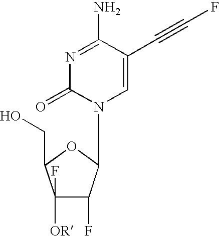

or its pharmaceutically acceptable salt or prodrug thereof or its use as further described herein is provided.

In another embodiment of the present invention, a β-D or β-L nucleoside of the formula:

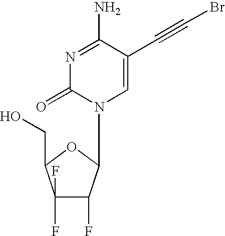

or its pharmaceutically acceptable salt or prodrug thereof or its use as further described herein is provided.

In another embodiment of the present invention, a β-D or β-L nucleoside of the formula:

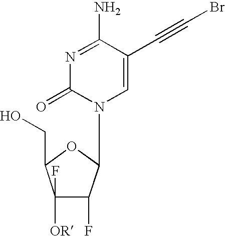

or its pharmaceutically acceptable salt or prodrug thereof or its use as further described herein is provided.

In another embodiment of the present invention, a β-D or β-L nucleoside of the formula:

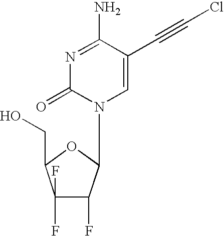

or its pharmaceutically acceptable salt or prodrug thereof or its use as further described herein is provided.

In another embodiment of the present invention, a β-D or β-L nucleoside of the formula:

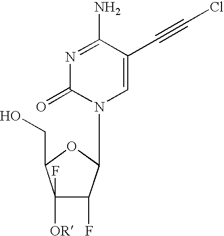

or its pharmaceutically acceptable salt or prodrug thereof or its use as further described herein is provided.

In another embodiment of the present invention, a β-D or β-L nucleoside of the formula:

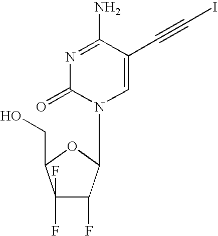

or its pharmaceutically acceptable salt or prodrug thereof or its use as further described herein is provided.

In another embodiment of the present invention, a β-D or β-L nucleoside of the formula:

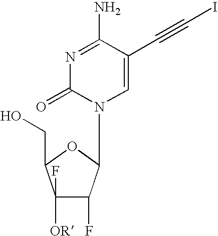

or its pharmaceutically acceptable salt or prodrug thereof or its use as further described herein is provided.

In another embodiment of the present invention, a β-D or β-L nucleoside of the formula:

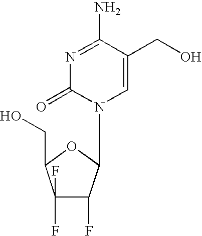

or its pharmaceutically acceptable salt or prodrug thereof or its use as further described herein is provided.

In another embodiment of the present invention, a β-D or β-L nucleoside of the formula:

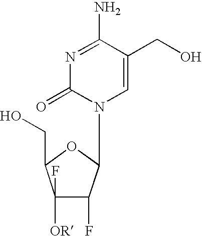

or its pharmaceutically acceptable salt or prodrug thereof or its use as further described herein is provided.

In another embodiment of the present invention, a β-D or β-L nucleoside of the formula:

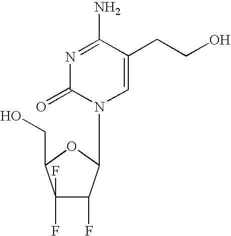

or its pharmaceutically acceptable salt or prodrug thereof or its use as further described herein is provided.

In another embodiment of the present invention, a β-D or β-L nucleoside of the formula:

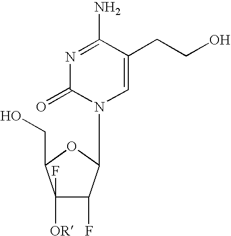

or its pharmaceutically acceptable salt or prodrug thereof or its use as further described herein is provided.

In another embodiment of the present invention, a β-D or β-L nucleoside of the formula:

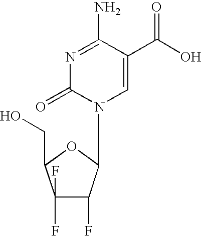

or its pharmaceutically acceptable salt or prodrug thereof or its use as further described herein is provided.

In another embodiment of the present invention, a β-D or β-L nucleoside of the formula:

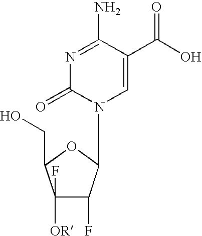

or its pharmaceutically acceptable salt or prodrug thereof or its use as further described herein is provided.

In another embodiment of the present invention, a β-D or β-L nucleoside of the formula:

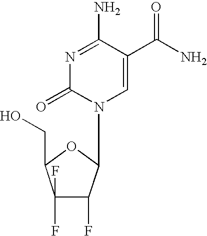

or its pharmaceutically acceptable salt or prodrug thereof or its use as further described herein is provided.

In another embodiment of the present invention, a β-D or β-L nucleoside of the formula:

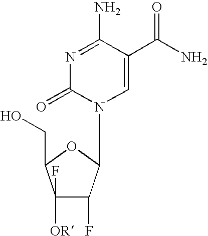

or its pharmaceutically acceptable salt or prodrug thereof or its use as further described herein is provided.

In another embodiment of the present invention, a β-D or β-L nucleoside of the formula:

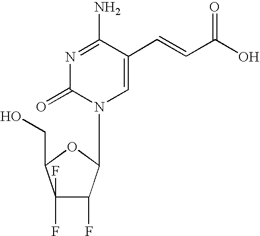

or its pharmaceutically acceptable salt or prodrug thereof or its use as further described herein is provided.

In another embodiment of the present invention, a β-D or β-L nucleoside of the formula:

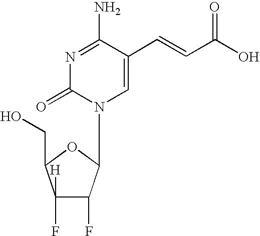

or its pharmaceutically acceptable salt or prodrug thereof or its use as further described herein is provided.

In another embodiment of the present invention, a β-D or β-L nucleoside of the formula:

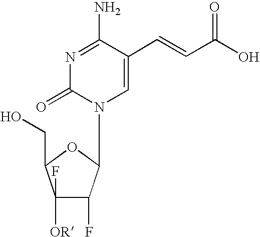

or its pharmaceutically acceptable salt or prodrug thereof or its use as further described herein is provided.

In another embodiment of the present invention, a β-D or β-L nucleoside of the formula:

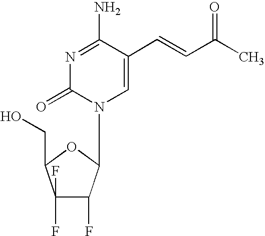

or its pharmaceutically acceptable salt or prodrug thereof or its use as further described herein is provided.

In another embodiment of the present invention, a β-D or β-L nucleoside of the formula:

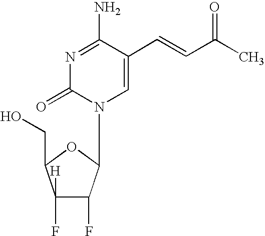

or its pharmaceutically acceptable salt or prodrug thereof or its use as further described herein is provided.

In another embodiment of the present invention, a β-D or β-L nucleoside of the formula:

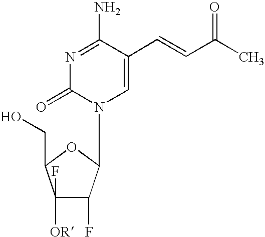

or its pharmaceutically acceptable salt or prodrug thereof or its use as further described herein is provided.

In another embodiment of the present invention, a β-D or β-L nucleoside of the formula:

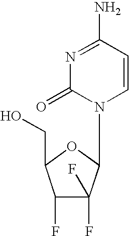

or its pharmaceutically acceptable salt or prodrug thereof or its use as further described herein is provided.

In another embodiment of the present invention, a β-D or β-L nucleoside of the formula:

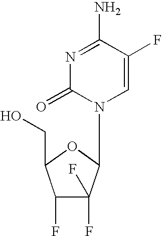

or its pharmaceutically acceptable salt or prodrug thereof or its use as further described herein is provided.

In another embodiment of the present invention, a β-D or β-L nucleoside of the formula:

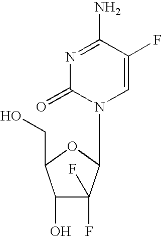

or its pharmaceutically acceptable salt or prodrug thereof or its use as further described herein is provided.

In another embodiment of the present invention, a β-D or β-L nucleoside of the formula:

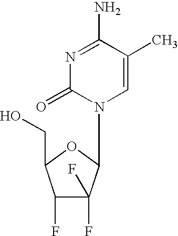

or its pharmaceutically acceptable salt or prodrug thereof or its use as further described herein is provided.

In another embodiment of the present invention, a β-D or β-L nucleoside of the formula:

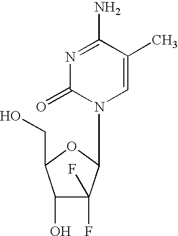

or its pharmaceutically acceptable salt or prodrug thereof or its use as further described herein is provided.

In another embodiment of the present invention, a β-D or β-L nucleoside of the formula:

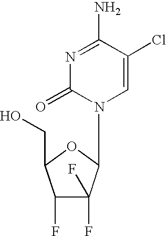

or its pharmaceutically acceptable salt or prodrug thereof or its use as further described herein is provided.

In another embodiment of the present invention, a β-D or β-L nucleoside of the formula:

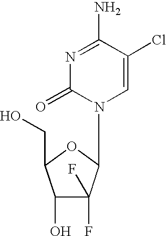

or its pharmaceutically acceptable salt or prodrug thereof or its use as further described herein is provided.

In another embodiment of the present invention, a β-D or β-L nucleoside of the formula:

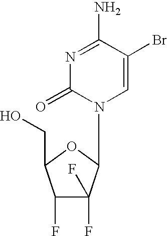

or its pharmaceutically acceptable salt or prodrug thereof or its use as further described herein is provided.

In another embodiment of the present invention, a β-D or β-L nucleoside of the formula:

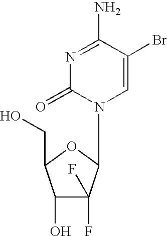

or its pharmaceutically acceptable salt or prodrug thereof or its use as further described herein is provided.

In another embodiment of the present invention, a β-D or β-L nucleoside of the formula:

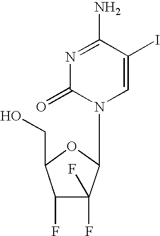

or its pharmaceutically acceptable salt or prodrug thereof or its use as further described herein is provided.

In another embodiment of the present invention, a β-D or β-L nucleoside of the formula:

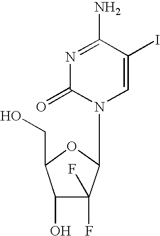

or its pharmaceutically acceptable salt or prodrug thereof or its use as further described herein is provided.

In another embodiment of the present invention, a β-D or β-L nucleoside of the formula:

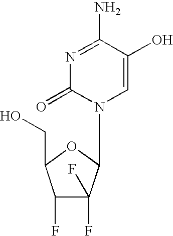

or its pharmaceutically acceptable salt or prodrug thereof or its use as further described herein is provided.

In another embodiment of the present invention, a β-D or β-L nucleoside of the formula:

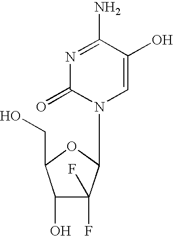

or its pharmaceutically acceptable salt or prodrug thereof or its use as further described herein is provided.

In another embodiment of the present invention, a β-D or β-L nucleoside of the formula:

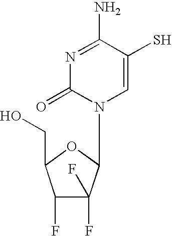

or its pharmaceutically acceptable salt or prodrug thereof or its use as further described herein is provided.

In another embodiment of the present invention, a β-D or β-L nucleoside of the formula:

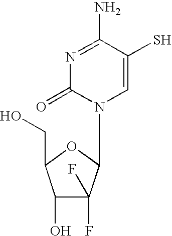

or its pharmaceutically acceptable salt or prodrug thereof or its use as further described herein is provided.

In another embodiment of the present invention, a β-D or β-L nucleoside of the formula:

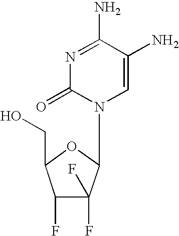

or its pharmaceutically acceptable salt or prodrug thereof or its use as further described herein is provided.

In another embodiment of the present invention, a β-D or β-L nucleoside of the formula:

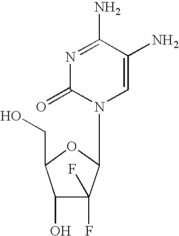

or its pharmaceutically acceptable salt or prodrug thereof or its use as further described herein is provided.

In another embodiment of the present invention, a β-D or β-L nucleoside of the formula:

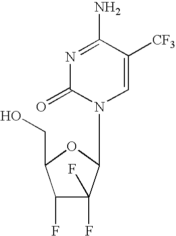

or its pharmaceutically acceptable salt or prodrug thereof or its use as further described herein is provided.

In another embodiment of the present invention, a β-D or β-L nucleoside of the formula:

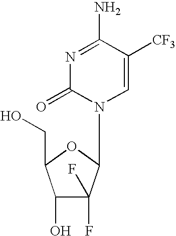

or its pharmaceutically acceptable salt or prodrug thereof or its use as further described herein is provided.

In another embodiment of the present invention, a β-D or β-L nucleoside of the formula:

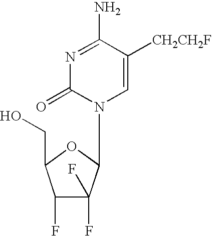

or its pharmaceutically acceptable salt or prodrug thereof or its use as further described herein is provided.

In another embodiment of the present invention, a β-D or β-L nucleoside of the formula:

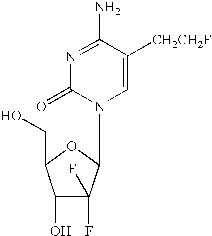

or its pharmaceutically acceptable salt or prodrug thereof or its use as further described herein is provided.

In another embodiment of the present invention, a β-D or β-L nucleoside of the formula:

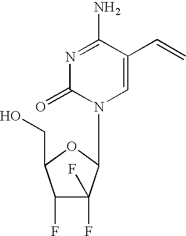

or its pharmaceutically acceptable salt or prodrug thereof or its use as further described herein is provided.

In another embodiment of the present invention, a β-D or β-L nucleoside of the formula:

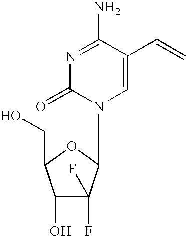

or its pharmaceutically acceptable salt or prodrug thereof or its use as further described herein is provided.

In another embodiment of the present invention, a β-D or β-L nucleoside of the formula:

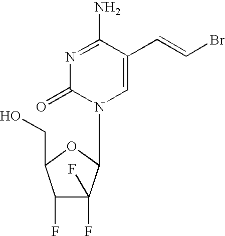

or its pharmaceutically acceptable salt or prodrug thereof or its use as further described herein is provided.

In another embodiment of the present invention, a β-D or β-L nucleoside of the formula:

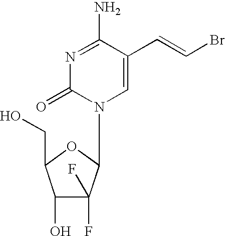

or its pharmaceutically acceptable salt or prodrug thereof or its use as further described herein is provided.

In another embodiment of the present invention, a β-D or β-L nucleoside of the formula:

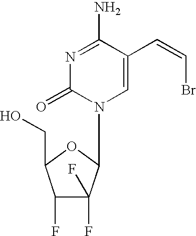

or its pharmaceutically acceptable salt or prodrug thereof or its use as further described herein is provided.

In another embodiment of the present invention, a β-D or β-L nucleoside of the formula:

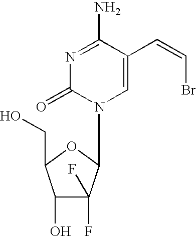

or its pharmaceutically acceptable salt or prodrug thereof or its use as further described herein is provided.

In another embodiment of the present invention, a β-D or β-L nucleoside of the formula:

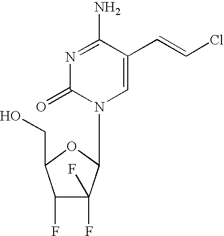

or its pharmaceutically acceptable salt or prodrug thereof or its use as further described herein is provided.

In another embodiment of the present invention, a β-D or β-L nucleoside of the formula:

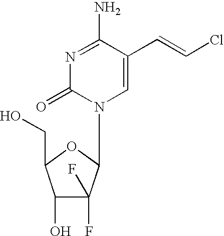

or its pharmaceutically acceptable salt or prodrug thereof or its use as further described herein is provided.

In another embodiment of the present invention, a β-D or β-L nucleoside of the formula:

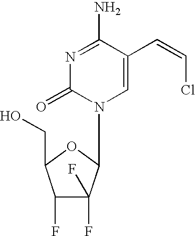

or its pharmaceutically acceptable salt or prodrug thereof or its use as further described herein is provided.

In another embodiment of the present invention, a β-D or β-L nucleoside of the formula:

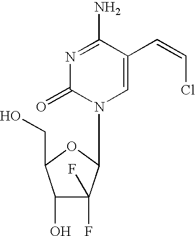

or its pharmaceutically acceptable salt or prodrug thereof or its use as further described herein is provided.

In another embodiment of the present invention, a β-D or β-L nucleoside of the formula:

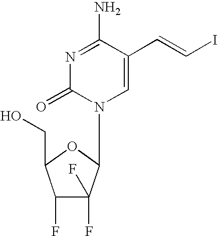

or its pharmaceutically acceptable salt or prodrug thereof or its use as further described herein is provided.

In another embodiment of the present invention, a β-D or β-L nucleoside of the formula:

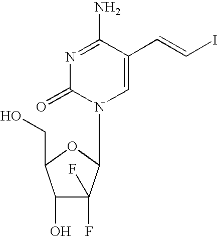

or its pharmaceutically acceptable salt or prodrug thereof or its use as further described herein is provided.

In another embodiment of the present invention, a β-D or β-L nucleoside of the formula:

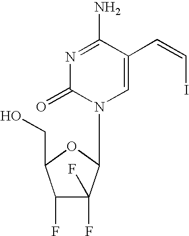

or its pharmaceutically acceptable salt or prodrug thereof or its use as further described herein is provided.

In another embodiment of the present invention, a β-D or β-L nucleoside of the formula:

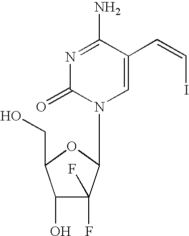

or its pharmaceutically acceptable salt or prodrug thereof or its use as further described herein is provided.

In another embodiment of the present invention, a β-D or β-L nucleoside of the formula:

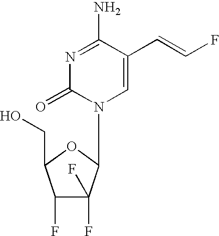

or its pharmaceutically acceptable salt or prodrug thereof or its use as further described herein is provided.

In another embodiment of the present invention, a β-D or β-L nucleoside of the formula:

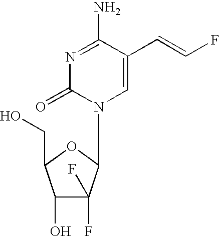

or its pharmaceutically acceptable salt or prodrug thereof or its use as further described herein is provided.

In another embodiment of the present invention, a β-D or β-L nucleoside of the formula:

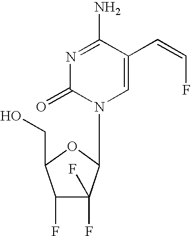

or its pharmaceutically acceptable salt or prodrug thereof or its use as further described herein is provided.

In another embodiment of the present invention, a β-D or β-L nucleoside of the formula:

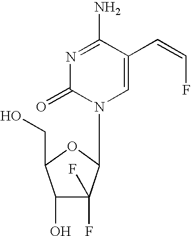

or its pharmaceutically acceptable salt or prodrug thereof or its use as further described herein is provided.

In another embodiment of the present invention, a β-D or β-L nucleoside of the formula:

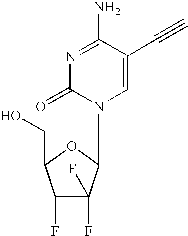

or its pharmaceutically acceptable salt or prodrug thereof or its use as further described herein is provided.

In another embodiment of the present invention, a β-D or β-L nucleoside of the formula:

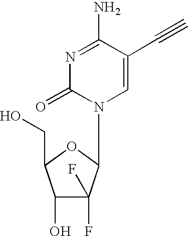

or its pharmaceutically acceptable salt or prodrug thereof or its use as further described herein is provided.

In another embodiment of the present invention, a β-D or β-L nucleoside of the formula:

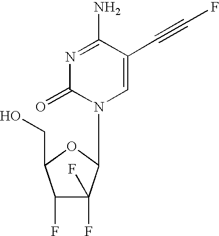

or its pharmaceutically acceptable salt or prodrug thereof or its use as further described herein is provided.

In another embodiment of the present invention, a β-D or β-L nucleoside of the formula:

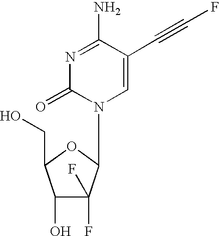

or its pharmaceutically acceptable salt or prodrug thereof or its use as further described herein is provided.

In another embodiment of the present invention, a β-D or β-L nucleoside of the formula:

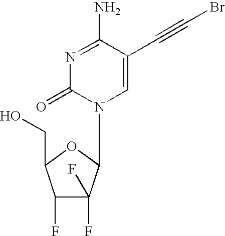

or its pharmaceutically acceptable salt or prodrug thereof or its use as further described herein is provided.

In another embodiment of the present invention, a β-D or β-L nucleoside of the formula:

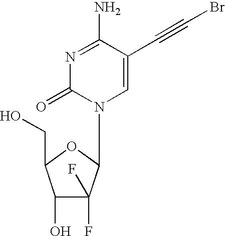

or its pharmaceutically acceptable salt or prodrug thereof or its use as further described herein is provided.

In another embodiment of the present invention, a β-D or β-L nucleoside of the formula:

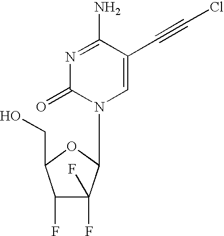

or its pharmaceutically acceptable salt or prodrug thereof or its use as further described herein is provided.

In another embodiment of the present invention, a β-D or β-L nucleoside of the formula:

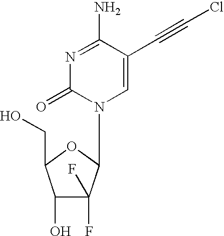

or its pharmaceutically acceptable salt or prodrug thereof or its use as further described herein is provided.

In another embodiment of the present invention, a β-D or β-L nucleoside of the formula:

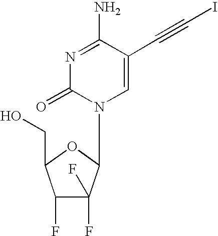

or its pharmaceutically acceptable salt or prodrug thereof or its use as further described herein is provided.

In another embodiment of the present invention, a β-D or β-L nucleoside of the formula:

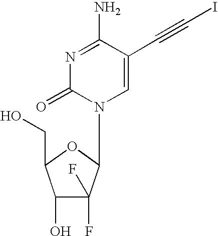

or its pharmaceutically acceptable salt or prodrug thereof or its use as further described herein is provided.

In another embodiment of the present invention, a β-D or β-L nucleoside of the formula:

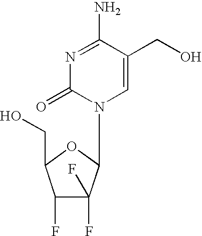

or its pharmaceutically acceptable salt or prodrug thereof or its use as further described herein is provided.

In another embodiment of the present invention, a β-D or β-L nucleoside of the formula:

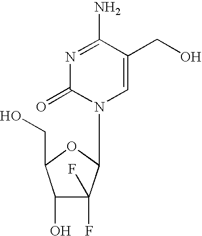

or its pharmaceutically acceptable salt or prodrug thereof or its use as further described herein is provided.

In another embodiment of the present invention, a β-D or β-L nucleoside of the formula:

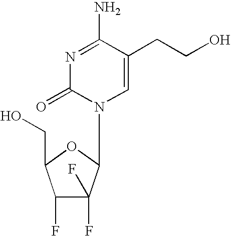

or its pharmaceutically acceptable salt or prodrug thereof or its use as further described herein is provided.

In another embodiment of the present invention, a β-D or β-L nucleoside of the formula:

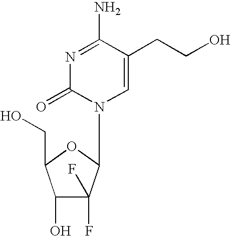

or its pharmaceutically acceptable salt or prodrug thereof or its use as further described herein is provided.

In another embodiment of the present invention, a β-D or β-L nucleoside of the formula:

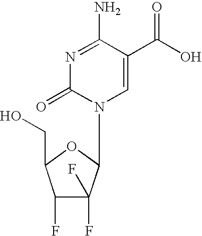

or its pharmaceutically acceptable salt or prodrug thereof or its use as further described herein is provided.

In another embodiment of the present invention, a β-D or β-L nucleoside of the formula:

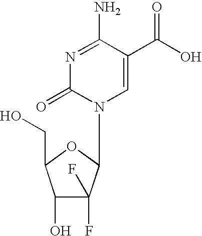

or its pharmaceutically acceptable salt or prodrug thereof or its use as further described herein is provided.

In another embodiment of the present invention, a β-D or β-L nucleoside of the formula:

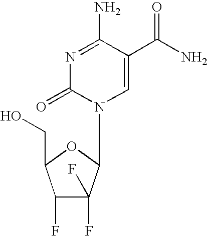

or its pharmaceutically acceptable salt or prodrug thereof or its use as further described herein is provided.

In another embodiment of the present invention, a β-D or β-L nucleoside of the formula:

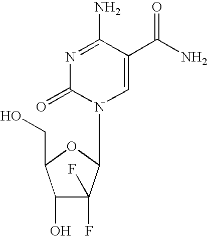

or its pharmaceutically acceptable salt or prodrug thereof or its use as further described herein is provided.

In another embodiment of the present invention, a β-D or β-L nucleoside of the formula:

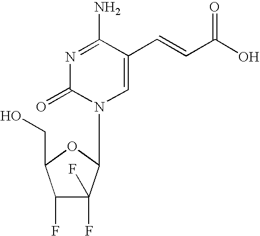

or its pharmaceutically acceptable salt or prodrug thereof or its use as further described herein is provided.

In another embodiment of the present invention, a β-D or β-L nucleoside of the formula:

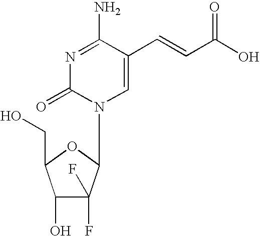

or its pharmaceutically acceptable salt or prodrug thereof or its use as further described herein is provided.

In another embodiment of the present invention, a β-D or β-L nucleoside of the formula:

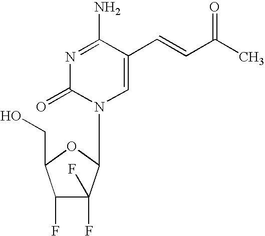

or its pharmaceutically acceptable salt or prodrug thereof or its use as further described herein is provided.

In another embodiment of the present invention, a β-D or β-L nucleoside of the formula:

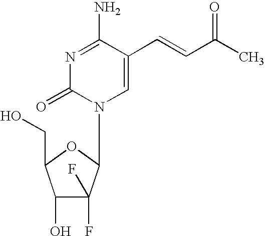

or its pharmaceutically acceptable salt or prodrug thereof or its use as further described herein is provided.

In another embodiment of the present invention, a β-D or β-L nucleoside of the formula:

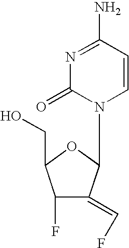

or its pharmaceutically acceptable salt or prodrug thereof or its use as further described herein is provided.

In another embodiment of the present invention, a β-D or β-L nucleoside of the formula:

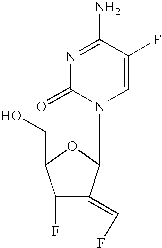

or its pharmaceutically acceptable salt or prodrug thereof or its use as further described herein is provided.

In another embodiment of the present invention, a β-D or β-L nucleoside of the formula:

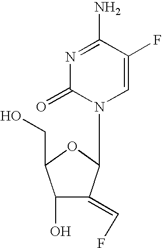

or its pharmaceutically acceptable salt or prodrug thereof or its use as further described herein is provided.

In another embodiment of the present invention, a β-D or β-L nucleoside of the formula:

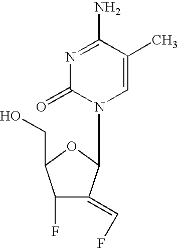

or its pharmaceutically acceptable salt or prodrug thereof or its use as further described herein is provided.

In another embodiment of the present invention, a β-D or β-L nucleoside of the formula:

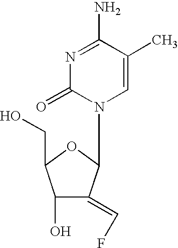

or its pharmaceutically acceptable salt or prodrug thereof or its use as further described herein is provided.

In another embodiment of the present invention, a β-D or β-L nucleoside of the formula:

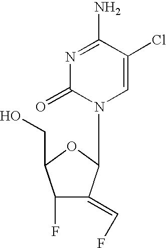

or its pharmaceutically acceptable salt or prodrug thereof or its use as further described herein is provided.

In another embodiment of the present invention, a β-D or β-L nucleoside of the formula:

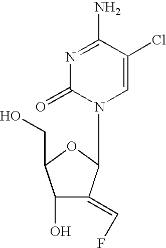

or its pharmaceutically acceptable salt or prodrug thereof or its use as further described herein is provided.

In another embodiment of the present invention, a β-D or β-L nucleoside of the formula:

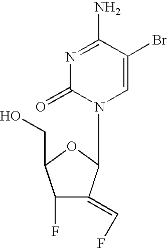

or its pharmaceutically acceptable salt or prodrug thereof or its use as further described herein is provided.

In another embodiment of the present invention, a β-D or β-L nucleoside of the formula:

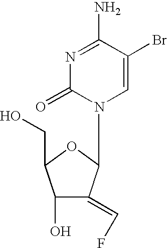

or its pharmaceutically acceptable salt or prodrug thereof or its use as further described herein is provided.

In another embodiment of the present invention, a β-D or β-L nucleoside of the formula:

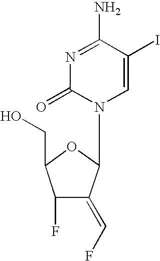

or its pharmaceutically acceptable salt or prodrug thereof or its use as further described herein is provided.

In another embodiment of the present invention, a β-D or β-L nucleoside of the formula:

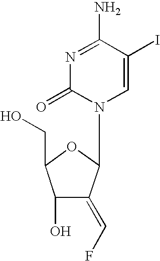

or its pharmaceutically acceptable salt or prodrug thereof or its use as further described herein is provided.

In another embodiment of the present invention, a β-D or β-L nucleoside of the formula:

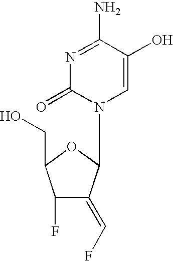

or its pharmaceutically acceptable salt or prodrug thereof or its use as further described herein is provided.

In another embodiment of the present invention, a β-D or β-L nucleoside of the formula:

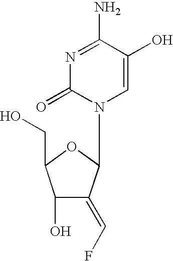

or its pharmaceutically acceptable salt or prodrug thereof or its use as further described herein is provided.

In another embodiment of the present invention, a β-D or β-L nucleoside of the formula:

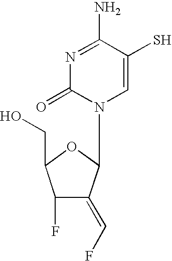

or its pharmaceutically acceptable salt or prodrug thereof or its use as further described herein is provided.

In another embodiment of the present invention, a β-D or β-L nucleoside of the formula:

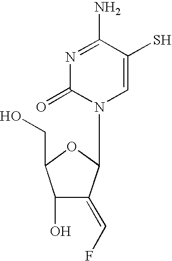

or its pharmaceutically acceptable salt or prodrug thereof or its use as further described herein is provided.

In another embodiment of the present invention, a β-D or β-L nucleoside of the formula:

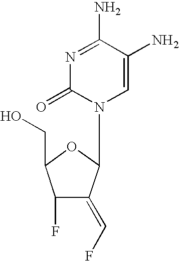

or its pharmaceutically acceptable salt or prodrug thereof or its use as further described herein is provided.

In another embodiment of the present invention, a β-D or β-L nucleoside of the formula:

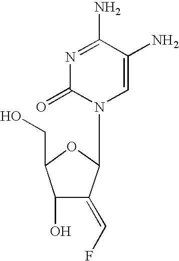

or its pharmaceutically acceptable salt or prodrug thereof or its use as further described herein is provided.

In another embodiment of the present invention, a β-D or β-L nucleoside of the formula:

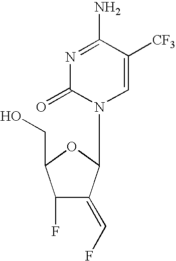

or its pharmaceutically acceptable salt or prodrug thereof or its use as further described herein is provided.

In another embodiment of the present invention, a β-D or β-L nucleoside of the formula:

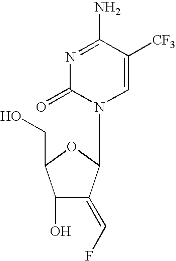

or its pharmaceutically acceptable salt or prodrug thereof or its use as further described herein is provided.

In another embodiment of the present invention, a β-D or β-L nucleoside of the formula:

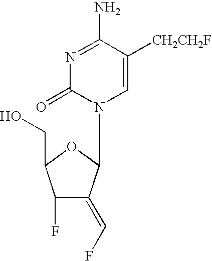

or its pharmaceutically acceptable salt or prodrug thereof or its use as further described herein is provided.

In another embodiment of the present invention, a β-D or β-L nucleoside of the formula:

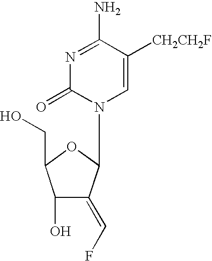

or its pharmaceutically acceptable salt or prodrug thereof or its use as further described herein is provided.

In another embodiment of the present invention, a β-D or β-L nucleoside of the formula:

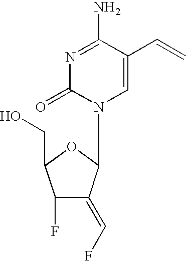

or its pharmaceutically acceptable salt or prodrug thereof or its use as further described herein is provided.

In another embodiment of the present invention, a β-D or β-L nucleoside of the formula:

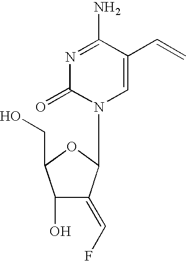

or its pharmaceutically acceptable salt or prodrug thereof or its use as further described herein is provided.

In another embodiment of the present invention, a β-D or β-L nucleoside of the formula:

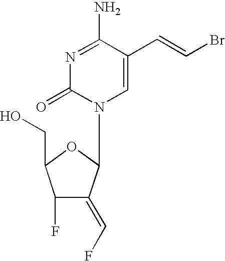

or its pharmaceutically acceptable salt or prodrug thereof or its use as further described herein is provided.

In another embodiment of the present invention, a β-D or β-L nucleoside of the formula:

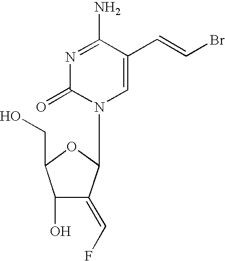

or its pharmaceutically acceptable salt or prodrug thereof or its use as further described herein is provided.

In another embodiment of the present invention, a β-D or β-L nucleoside of the formula:

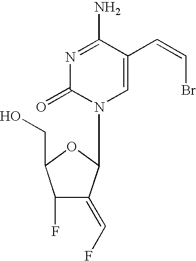

or its pharmaceutically acceptable salt or prodrug thereof or its use as further described herein is provided.

In another embodiment of the present invention, a β-D or β-L nucleoside of the formula:

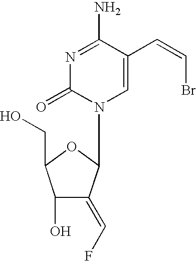

or its pharmaceutically acceptable salt or prodrug thereof or its use as further described herein is provided.

In another embodiment of the present invention, a β-D or β-L nucleoside of the formula:

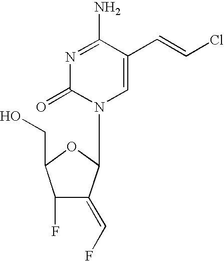

or its pharmaceutically acceptable salt or prodrug thereof or its use as further described herein is provided.

In another embodiment of the present invention, a β-D or β-L nucleoside of the formula:

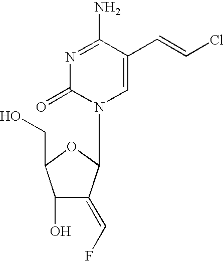

or its pharmaceutically acceptable salt or prodrug thereof or its use as further described herein is provided.

In another embodiment of the present invention, a β-D or β-L nucleoside of the formula:

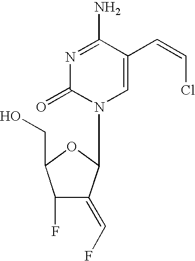

or its pharmaceutically acceptable salt or prodrug thereof or its use as further described herein is provided.

In another embodiment of the present invention, a β-D or β-L nucleoside of the formula:

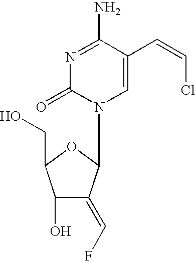

or its pharmaceutically acceptable salt or prodrug thereof or its use as further described herein is provided.

In another embodiment of the present invention, a β-D or β-L nucleoside of the formula:

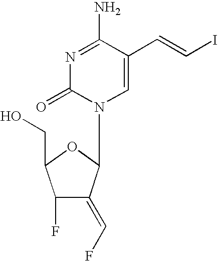

or its pharmaceutically acceptable salt or prodrug thereof or its use as further described herein is provided.

In another embodiment of the present invention, a β-D or β-L nucleoside of the formula:

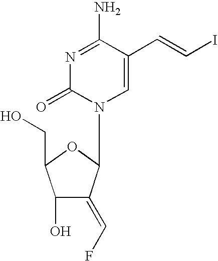

or its pharmaceutically acceptable salt or prodrug thereof or its use as further described herein is provided.

In another embodiment of the present invention, a β-D or βL nucleoside of the formula:

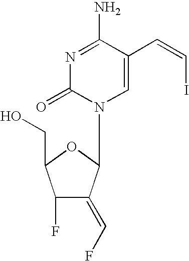

or its pharmaceutically acceptable salt or prodrug thereof or its use as further described herein is provided.

In another embodiment of the present invention, a β-D or β-L nucleoside of the formula:

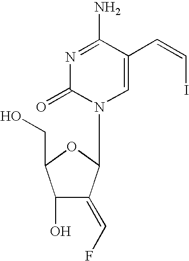

or its pharmaceutically acceptable salt or prodrug thereof or its use as further described herein is provided.

In another embodiment of the present invention, a β-D or β-L nucleoside of the formula:

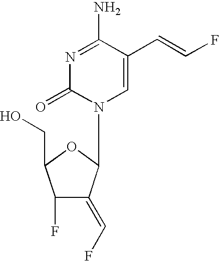

or its pharmaceutically acceptable salt or prodrug thereof or its use as further described herein is provided.

In another embodiment of the present invention, a β-D or β-L nucleoside of the formula:

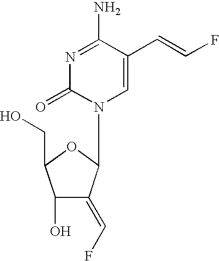

or its pharmaceutically acceptable salt or prodrug thereof or its use as further described herein is provided.

In another embodiment of the present invention, a β-D or β-L nucleoside of the formula:

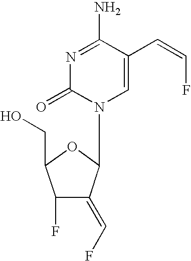

or its pharmaceutically acceptable salt or prodrug thereof or its use as further described herein is provided.

In another embodiment of the present invention, a β-D or β-L nucleoside of the formula:

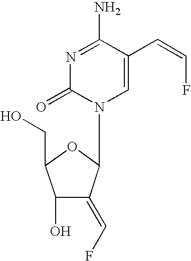

or its pharmaceutically acceptable salt or prodrug thereof or its use as further described herein is provided.

In another embodiment of the present invention, a β-D or β-L nucleoside of the formula:

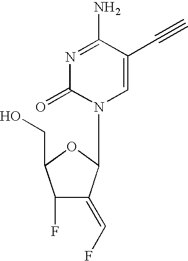

or its pharmaceutically acceptable salt or prodrug thereof or its use as further described herein is provided.

In another embodiment of the present invention, a β-D or β-L nucleoside of the formula:

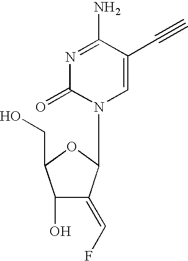

or its pharmaceutically acceptable salt or prodrug thereof or its use as further described herein is provided.

In another embodiment of the present invention, a β-D or β-L nucleoside of the formula:

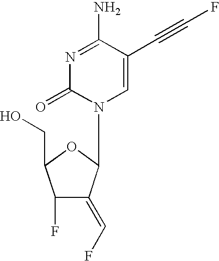

or its pharmaceutically acceptable salt or prodrug thereof or its use as further described herein is provided.

In another embodiment of the present invention, a β-D or β-L nucleoside of the formula:

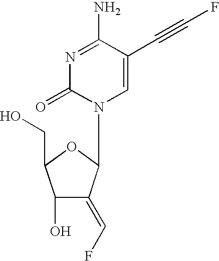

or its pharmaceutically acceptable salt or prodrug thereof or its use as further described herein is provided.

In another embodiment of the present invention, a β-D or β-L nucleoside of the formula:

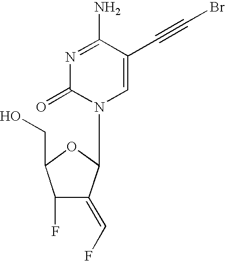

or its pharmaceutically acceptable salt or prodrug thereof or its use as further described herein is provided.

In another embodiment of the present invention, a β-D or β-L nucleoside of the formula:

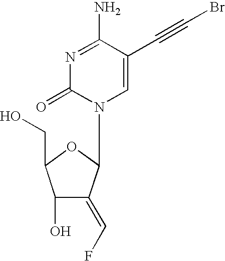

or its pharmaceutically acceptable salt or prodrug thereof or its use as further described herein is provided.

In another embodiment of the present invention, a β-D or β-L nucleoside of the formula:

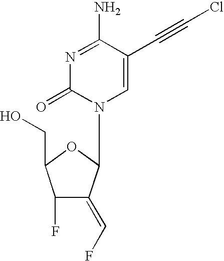

or its pharmaceutically acceptable salt or prodrug thereof or its use as further described herein is provided.

In another embodiment of the present invention, a β-D or β-L nucleoside of the formula:

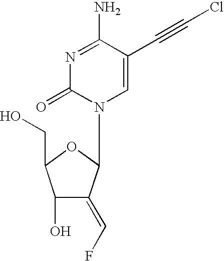

or its pharmaceutically acceptable salt or prodrug thereof or its use as further described herein is provided.

In another embodiment of the present invention, a β-D or β-L nucleoside of the formula:

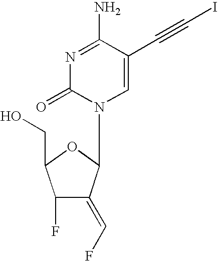

or its pharmaceutically acceptable salt or prodrug thereof or its use as further described herein is provided.

In another embodiment of the present invention, a β-D or β-L nucleoside of the formula:

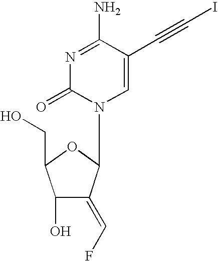

or its pharmaceutically acceptable salt or prodrug thereof or its use as further described herein is provided.

In another embodiment of the present invention, a β-D or β-L nucleoside of the formula:

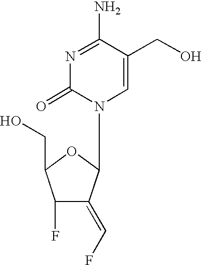

or its pharmaceutically acceptable salt or prodrug thereof or its use as further described herein is provided.

In another embodiment of the present invention, a β-D or β-L nucleoside of the formula:

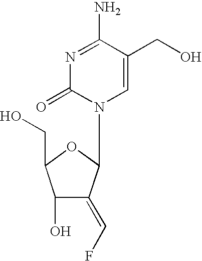

or its pharmaceutically acceptable salt or prodrug thereof or its use as further described herein is provided.

In another embodiment of the present invention, a β-D or β-L nucleoside of the formula:

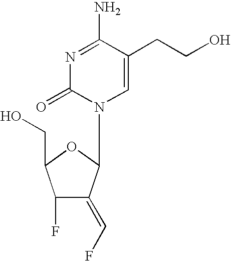

or its pharmaceutically acceptable salt or prodrug thereof or its use as further described herein is provided.

In another embodiment of the present invention, a β-D or β-L nucleoside of the formula:

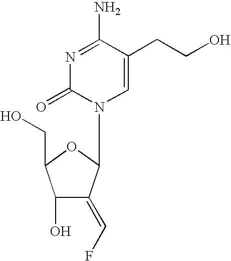

or its pharmaceutically acceptable salt or prodrug thereof or its use as further described herein is provided.

In another embodiment of the present invention, a β-D or β-L nucleoside of the formula:

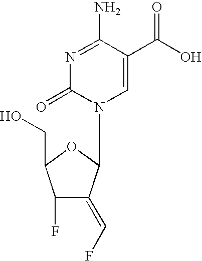

or its pharmaceutically acceptable salt or prodrug thereof or its use as further described herein is provided.

In another embodiment of the present invention, a β-D or β-L nucleoside of the formula:

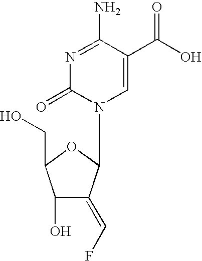

or its pharmaceutically acceptable salt or prodrug thereof or its use as further described herein is provided.

In another embodiment of the present invention, a β-D or β-L nucleoside of the formula:

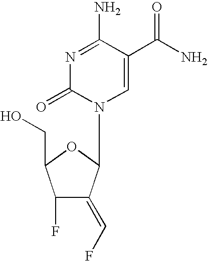

or its pharmaceutically acceptable salt or prodrug thereof or its use as further described herein is provided.

In another embodiment of the present invention, a β-D or β-L nucleoside of the formula:

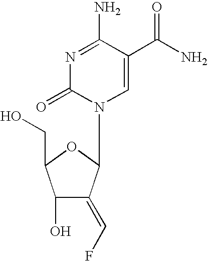

or its pharmaceutically acceptable salt or prodrug thereof or its use as further described herein is provided.

In another embodiment of the present invention, a β-D or β-L nucleoside of the formula:

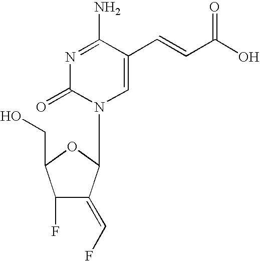

or its pharmaceutically acceptable salt or prodrug thereof or its use as further described herein is provided.

In another embodiment of the present invention, a β-D or β-L nucleoside of the formula:

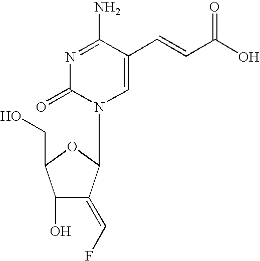

or its pharmaceutically acceptable salt or prodrug thereof or its use as further described herein is provided.

In another embodiment of the present invention, a β-D or β-L nucleoside of the formula:

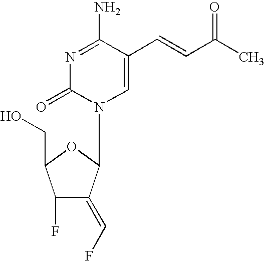

or its pharmaceutically acceptable salt or prodrug thereof or its use as further described herein is provided.

In another embodiment of the present invention, a β-D or β-L nucleoside of the formula:

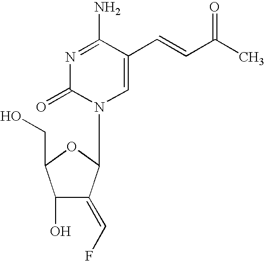

or its pharmaceutically acceptable salt or prodrug thereof or its use as further described herein is provided.

In another embodiment of the present invention, a β-D or β-L nucleoside of the formula:

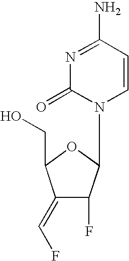

or its pharmaceutically acceptable salt or prodrug thereof or its use as further described herein is provided.

In another embodiment of the present invention, a β-D or β-L nucleoside of the formula:

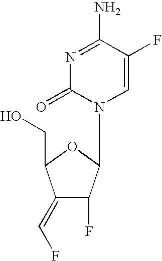

or its pharmaceutically acceptable salt or prodrug thereof or its use as further described herein is provided.

In another embodiment of the present invention, a β-D or β-L nucleoside of the formula:

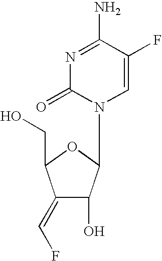

or its pharmaceutically acceptable salt or prodrug thereof or its use as further described herein is provided.

In another embodiment of the present invention, a β-D or β-L nucleoside of the formula:

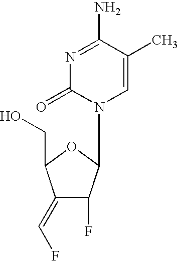

or its pharmaceutically acceptable salt or prodrug thereof or its use as further described herein is provided.

In another embodiment of the present invention, a β-D or β-L nucleoside of the formula:

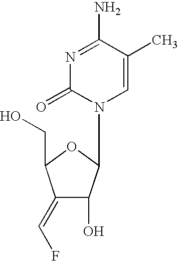

or its pharmaceutically acceptable salt or prodrug thereof or its use as further described herein is provided.

In another embodiment of the present invention, a β-D or β-L nucleoside of the formula:

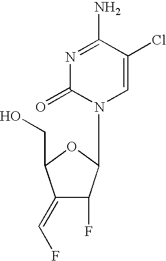

or its pharmaceutically acceptable salt or prodrug thereof or its use as further described herein is provided.

In another embodiment of the present invention, a β-D or β-L nucleoside of the formula:

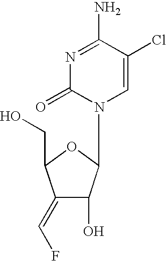

or its pharmaceutically acceptable salt or prodrug thereof or its use as further described herein is provided.

In another embodiment of the present invention, a β-D or β-L nucleoside of the formula:

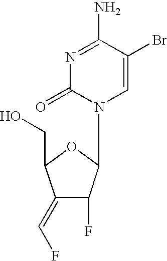

or its pharmaceutically acceptable salt or prodrug thereof or its use as further described herein is provided.

In another embodiment of the present invention, a β-D or β-L nucleoside of the formula:

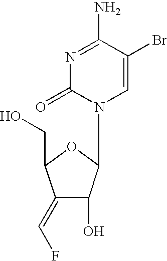

or its pharmaceutically acceptable salt or prodrug thereof or its use as further described herein is provided.

In another embodiment of the present invention, a β-D or β-L nucleoside of the formula:

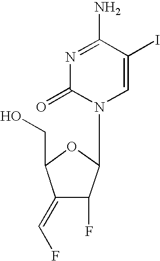

or its pharmaceutically acceptable salt or prodrug thereof or its use as further described herein is provided.

In another embodiment of the present invention, a β-D or β-L nucleoside of the formula:

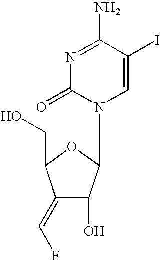

or its pharmaceutically acceptable salt or prodrug thereof or its use as further described herein is provided.

In another embodiment of the present invention, a β-D or β-L nucleoside of the formula:

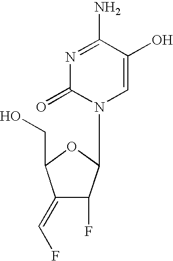

or its pharmaceutically acceptable salt or prodrug thereof or its use as further described herein is provided.

In another embodiment of the present invention, a β-D or β-L nucleoside of the formula:

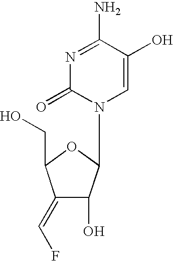

or its pharmaceutically acceptable salt or prodrug thereof or its use as further described herein is provided.

In another embodiment of the present invention, a β-D or β-L nucleoside of the formula:

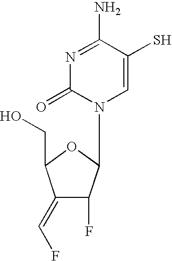

or its pharmaceutically acceptable salt or prodrug thereof or its use as further described herein is provided.

In another embodiment of the present invention, a β-D or β-L nucleoside of the formula:

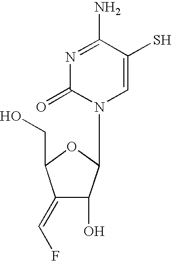

or its pharmaceutically acceptable salt or prodrug thereof or its use as further described herein is provided.

In another embodiment of the present invention, a β-D or β-L nucleoside of the formula:

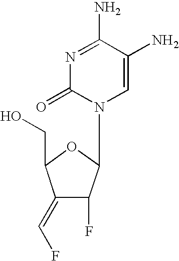

or its pharmaceutically acceptable salt or prodrug thereof or its use as further described herein is provided.

In another embodiment of the present invention, a β-D or β-L nucleoside of the formula:

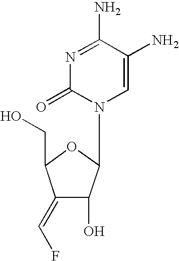

or its pharmaceutically acceptable salt or prodrug thereof or its use as further described herein is provided.

In another embodiment of the present invention, a β-D or β-L nucleoside of the formula:

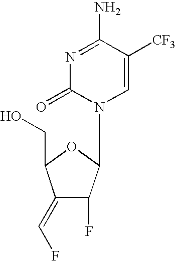

or its pharmaceutically acceptable salt or prodrug thereof or its use as further described herein is provided.

In another embodiment of the present invention, a β-D or β-L nucleoside of the formula:

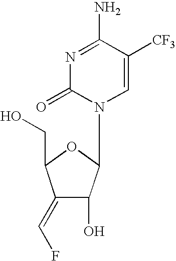

or its pharmaceutically acceptable salt or prodrug thereof or its use as further described herein is provided.

In another embodiment of the present invention, a β-D or β-L nucleoside of the formula:

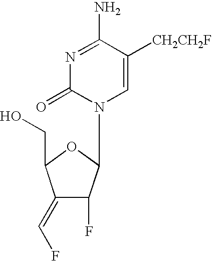

or its pharmaceutically acceptable salt or prodrug thereof or its use as further described herein is provided.

In another embodiment of the present invention, a β-D or β-L nucleoside of the formula:

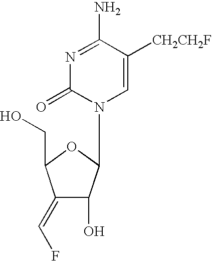

or its pharmaceutically acceptable salt or prodrug thereof or its use as further described herein is provided.

In another embodiment of the present invention, a β-D or β-L nucleoside of the formula:

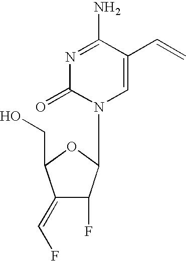

or its pharmaceutically acceptable salt or prodrug thereof or its use as further described herein is provided.

In another embodiment of the present invention, a β-D or β-L nucleoside of the formula:

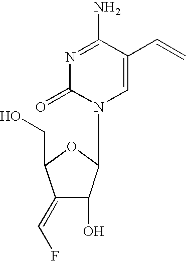

or its pharmaceutically acceptable salt or prodrug thereof or its use as further described herein is provided.

In another embodiment of the present invention, a β-D or β-L nucleoside of the formula:

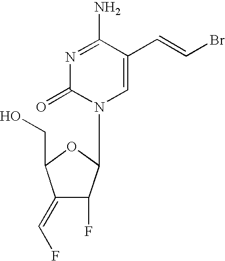

or its pharmaceutically acceptable salt or prodrug thereof or its use as further described herein is provided.

In another embodiment of the present invention, a β-D or β-L nucleoside of the formula:

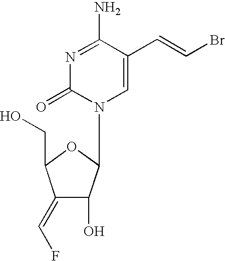

or its pharmaceutically acceptable salt or prodrug thereof or its use as further described herein is provided.

In another embodiment of the present invention, a β-D or β-L nucleoside of the formula:

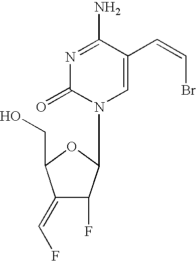

or its pharmaceutically acceptable salt or prodrug thereof or its use as further described herein is provided.

In another embodiment of the present invention, a β-D or β-L nucleoside of the formula:

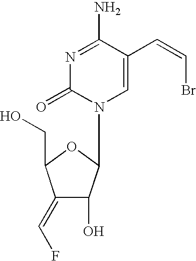

or its pharmaceutically acceptable salt or prodrug thereof or its use as further described herein is provided.

In another embodiment of the present invention, a β-D or β-L nucleoside of the formula:

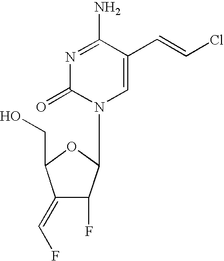

or its pharmaceutically acceptable salt or prodrug thereof or its use as further described herein is provided.

In another embodiment of the present invention, a β-D or β-L nucleoside of the formula:

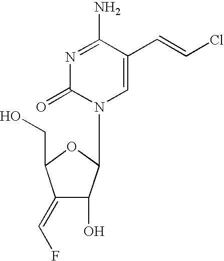

or its pharmaceutically acceptable salt or prodrug thereof or its use as further described herein is provided.

In another embodiment of the present invention, a β-D or β-L nucleoside of the formula:

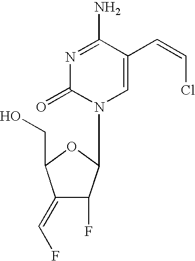

or its pharmaceutically acceptable salt or prodrug thereof or its use as further described herein is provided.

In another embodiment of the present invention, a β-D or β-L nucleoside of the formula:

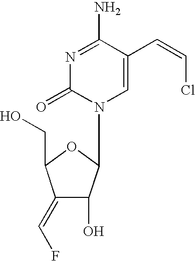

or its pharmaceutically acceptable salt or prodrug thereof or its use as further described herein is provided.

In another embodiment of the present invention, a β-D or β-L nucleoside of the formula:

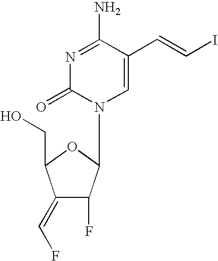

or its pharmaceutically acceptable salt or prodrug thereof or its use as further described herein is provided.

In another embodiment of the present invention, a β-D or β-L nucleoside of the formula:

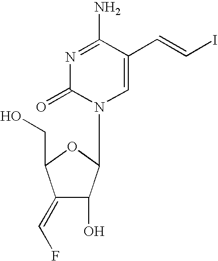

or its pharmaceutically acceptable salt or prodrug thereof or its use as further described herein is provided.

In another embodiment of the present invention, a β-D or β-L nucleoside of the formula:

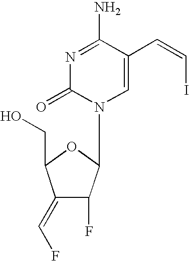

or its pharmaceutically acceptable salt or prodrug thereof or its use as further described herein is provided.

In another embodiment of the present invention, a β-D or β-L nucleoside of the formula:

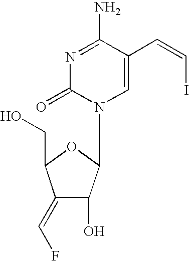

or its pharmaceutically acceptable salt or prodrug thereof or its use as further described herein is provided.

In another embodiment of the present invention, a β-D or β-L nucleoside of the formula:

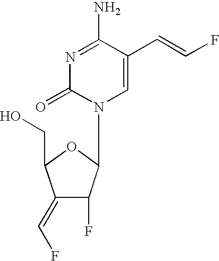

or its pharmaceutically acceptable salt or prodrug thereof or its use as further described herein is provided.

In another embodiment of the present invention, a β-D or β-L nucleoside of the formula:

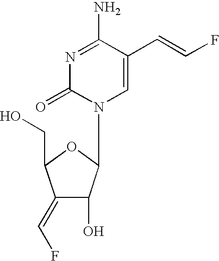

or its pharmaceutically acceptable salt or prodrug thereof or its use as further described herein is provided.

In another embodiment of the present invention, a β-D or β-L nucleoside of the formula:

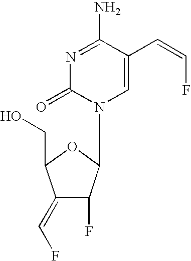

or its pharmaceutically acceptable salt or prodrug thereof or its use as further described herein is provided.

In another embodiment of the present invention, a β-D or β-L nucleoside of the formula:

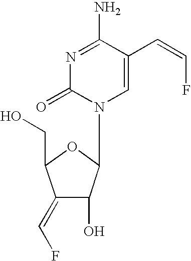

or its pharmaceutically acceptable salt or prodrug thereof or its use as further described herein is provided.

In another embodiment of the present invention, a β-D or β-L nucleoside of the formula:

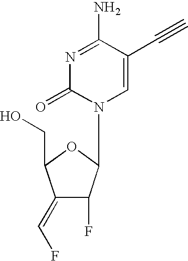

or its pharmaceutically acceptable salt or prodrug thereof or its use as further described herein is provided.

In another embodiment of the present invention, a β-D or β-L nucleoside of the formula:

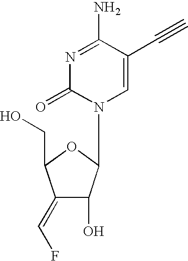

or its pharmaceutically acceptable salt or prodrug thereof or its use as further described herein is provided.

In another embodiment of the present invention, a β-D or β-L nucleoside of the formula:

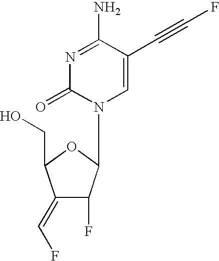

or its pharmaceutically acceptable salt or prodrug thereof or its use as further described herein is provided.

In another embodiment of the present invention, a β-D or β-L nucleoside of the formula:

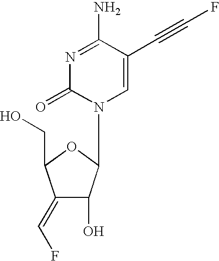

or its pharmaceutically acceptable salt or prodrug thereof or its use as further described herein is provided.

In another embodiment of the present invention, a β-D or β-L nucleoside of the formula:

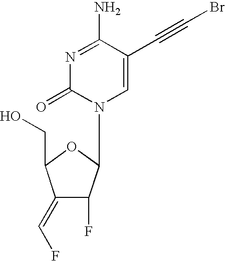

or its pharmaceutically acceptable salt or prodrug thereof or its use as further described herein is provided.

In another embodiment of the present invention, a β-D or β-L nucleoside of the formula:

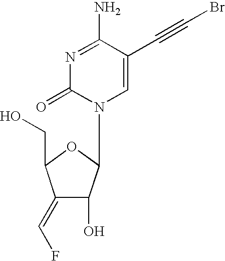

or its pharmaceutically acceptable salt or prodrug thereof or its use as further described herein is provided.

In another embodiment of the present invention, a β-D or β-L nucleoside of the formula:

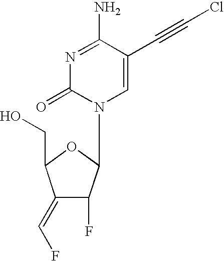

or its pharmaceutically acceptable salt or prodrug thereof or its use as further described herein is provided.

In another embodiment of the present invention, a β-D or β-L nucleoside of the formula:

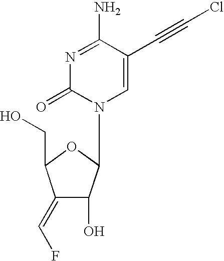

or its pharmaceutically acceptable salt or prodrug thereof or its use as further described herein is provided.

In another embodiment of the present invention, a β-D or β-L nucleoside of the formula:

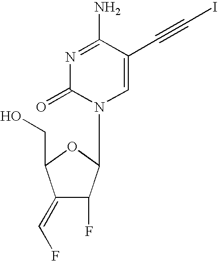

or its pharmaceutically acceptable salt or prodrug thereof or its use as further described herein is provided.

In another embodiment of the present invention, a β-D or β-L nucleoside of the formula:

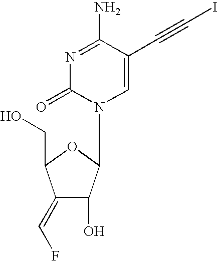

or its pharmaceutically acceptable salt or prodrug thereof or its use as further described herein is provided.

In another embodiment of the present invention, β-D or β-L nucleoside of the formula:

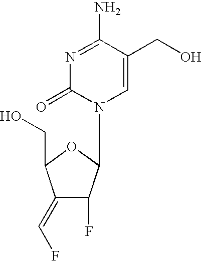

or its pharmaceutically acceptable salt or prodrug thereof or its use as further described herein is provided.

In another embodiment of the present invention, a β-D or β-L nucleoside of the formula:

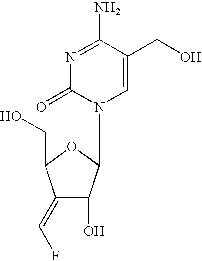

or its pharmaceutically acceptable salt or prodrug thereof or its use as further described herein is provided.

In another embodiment of the present invention, a β-D or β-L nucleoside of the formula:

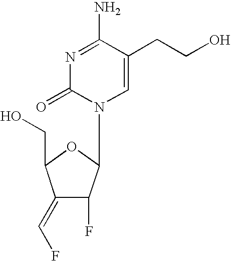

or its pharmaceutically acceptable salt or prodrug thereof or its use as further described herein is provided.

In another embodiment of the present invention, a β-D or β-L nucleoside of the formula:

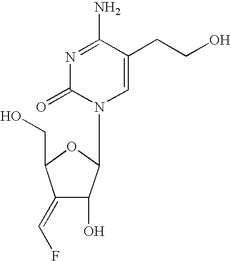

or its pharmaceutically acceptable salt or prodrug thereof or its use as further described herein is provided.

In another embodiment of the present invention, a β-D or β-L nucleoside of the formula:

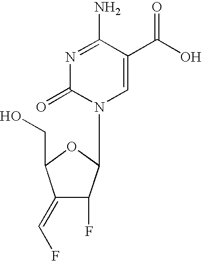

or its pharmaceutically acceptable salt or prodrug thereof or its use as further described herein is provided.

In another embodiment of the present invention, a β-D or β-L nucleoside of the formula:

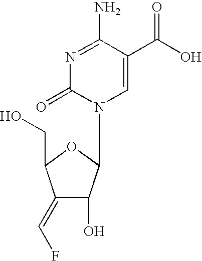

or its pharmaceutically acceptable salt or prodrug thereof or its use as further described herein is provided.

In another embodiment of the present invention, a β-D or β-L nucleoside of the formula:

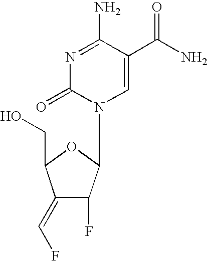

or its pharmaceutically acceptable salt or prodrug thereof or its use as further described herein is provided.

In another embodiment of the present invention, a β-D or β-L nucleoside of the formula:

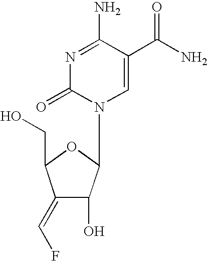

or its pharmaceutically acceptable salt or prodrug thereof or its use as further described herein is provided.

In another embodiment of the present invention, a β-D or β-L nucleoside of the formula:

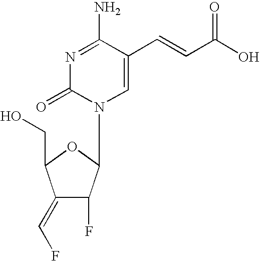

or its pharmaceutically acceptable salt or prodrug thereof or its use as further described herein is provided.

In another embodiment of the present invention, a β-D or β-L nucleoside of the formula:

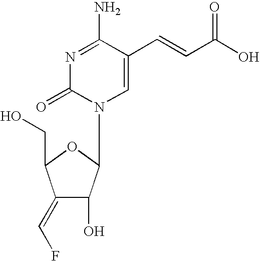

or its pharmaceutically acceptable salt or prodrug thereof or its use as further described herein is provided.

In another embodiment of the present invention, a β-D or β-L nucleoside of the formula:

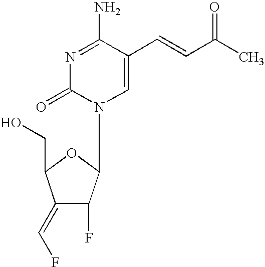

or its pharmaceutically acceptable salt or prodrug thereof or its use as further described herein is provided.

In another embodiment of the present invention, a β-D or β-L nucleoside of the formula:

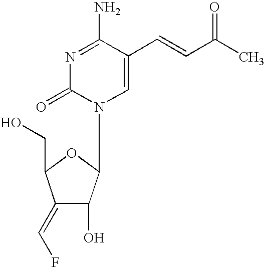

or its pharmaceutically acceptable salt or prodrug thereof or its use as further described herein is provided.

In another embodiment of the present invention, a β-D or β-L nucleoside of the formula:

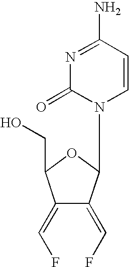

or its pharmaceutically acceptable salt or prodrug thereof or its use as further described herein is provided.

In another embodiment of the present invention, a β-D or β-L nucleoside of the formula:

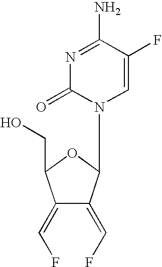

or its pharmaceutically acceptable salt or prodrug thereof or its use as further described herein is provided.

In another embodiment of the present invention, a β-D or β-L nucleoside of the formula:

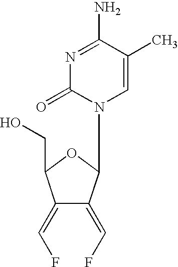

or its pharmaceutically acceptable salt or prodrug thereof or its use as further described herein is provided.

In another embodiment of the present invention, a β-D or β-L nucleoside of the formula:

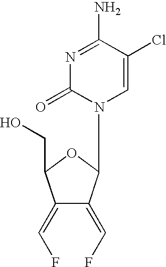

or its pharmaceutically acceptable salt or prodrug thereof or its use as further described herein is provided.

In another embodiment of the present invention, a β-D or β-L nucleoside of the formula:

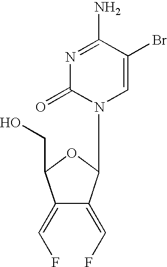

or its pharmaceutically acceptable salt or prodrug thereof or its use as further described herein is provided.

In another embodiment of the present invention, a β-D or β-L nucleoside of the formula:

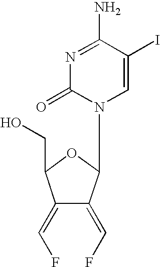

or its pharmaceutically acceptable salt or prodrug thereof or its use as further described herein is provided.

In another embodiment of the present invention, a β-D or β-L nucleoside of the formula:

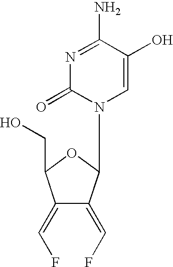

or its pharmaceutically acceptable salt or prodrug thereof or its use as further described herein is provided.

In another embodiment of the present invention, a β-D or β-L nucleoside of the formula:

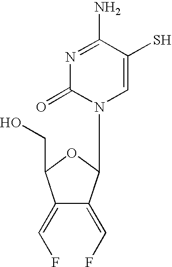

or its pharmaceutically acceptable salt or prodrug thereof or its use as further described herein is provided.

In another embodiment of the present invention, a β-D or β-L nucleoside of the formula:

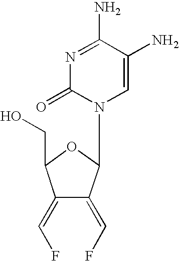

or its pharmaceutically acceptable salt or prodrug thereof or its use as further described herein is provided.

In another embodiment of the present invention, a β-D or β-L nucleoside of the formula:

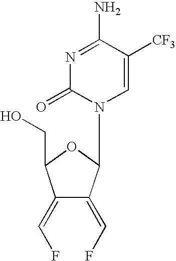

or its pharmaceutically acceptable salt or prodrug thereof or its use as further described herein is provided.

In another embodiment of the present invention, a β-D or β-L nucleoside of the formula:

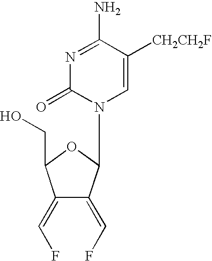

or its pharmaceutically acceptable salt or prodrug thereof or its use as further described herein is provided.

In another embodiment of the present invention, a β-D or β-L nucleoside of the formula:

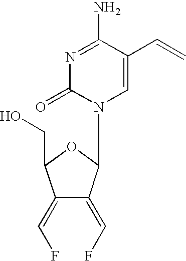

or its pharmaceutically acceptable salt or prodrug thereof or its use as further described herein is provided.

In another embodiment of the present invention, a β-D or β-L nucleoside of the formula:

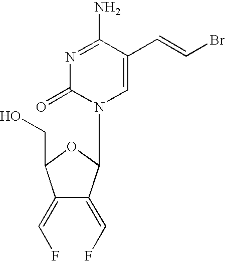

or its pharmaceutically acceptable salt or prodrug thereof or its use as further described herein is provided.

In another embodiment of the present invention, a β-D or β-L nucleoside of the formula:

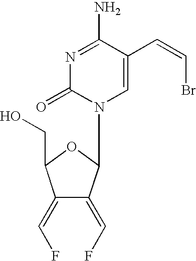

or its pharmaceutically acceptable salt or prodrug thereof or its use as further described herein is provided.

In another embodiment of the present invention, a β-D or β-L nucleoside of the formula:

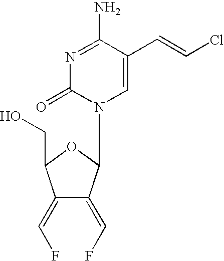

or its pharmaceutically acceptable salt or prodrug thereof or its use as further described herein is provided.

In another embodiment of the present invention, a β-D or β-L nucleoside of the formula:

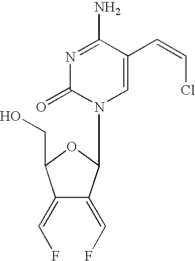

or its pharmaceutically acceptable salt or prodrug thereof or its use as further described herein is provided.

In another embodiment of the present invention, a β-D or β-L nucleoside of the formula:

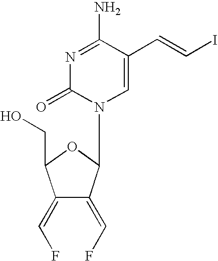

or its pharmaceutically acceptable salt or prodrug thereof or its use as further described herein is provided.

In another embodiment of the present invention, a β-D or β-L nucleoside of the formula:

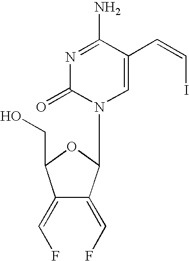

or its pharmaceutically acceptable salt or prodrug thereof or its use as further described herein is provided.

In another embodiment of the present invention, a β-D or β-L nucleoside of the formula:

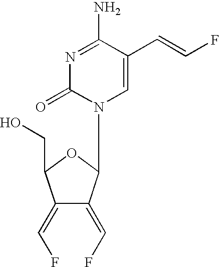

or its pharmaceutically acceptable salt or prodrug thereof or its use as further described herein is provided.

In another embodiment of the present invention, a β-D or β-L nucleoside of the formula:

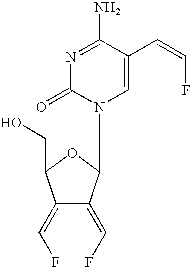

or its pharmaceutically acceptable salt or prodrug thereof or its use as further described herein is provided.

In another embodiment of the present invention, a β-D or β-L nucleoside of the formula:

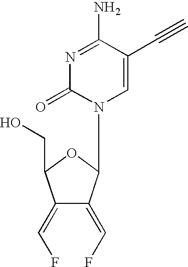

or its pharmaceutically acceptable salt or prodrug thereof or its use as further described herein is provided.

In another embodiment of the present invention, a β-D or β-L nucleoside of the formula:

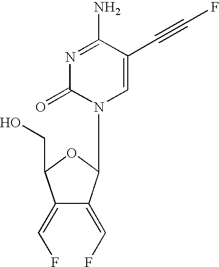

or its pharmaceutically acceptable salt or prodrug thereof or its use as further described herein is provided.

In another embodiment of the present invention, a β-D or β-L nucleoside of the formula:

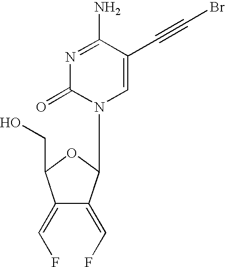

or its pharmaceutically acceptable salt or prodrug thereof or its use as further described herein is provided.

In another embodiment of the present invention, a β-D or β-L nucleoside of the formula:

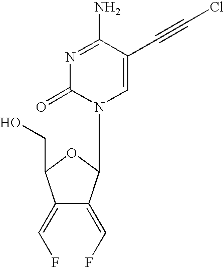

or its pharmaceutically acceptable salt or prodrug thereof or its use as further described herein is provided.

In another embodiment of the present invention, a β-D or β-L nucleoside of the formula:

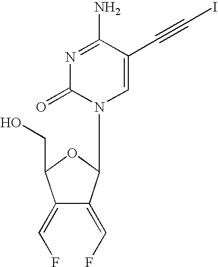

or its pharmaceutically acceptable salt or prodrug thereof or its use as further described herein is provided.

In another embodiment of the present invention, a β-D or β-L nucleoside of the formula:

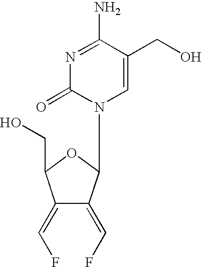

or its pharmaceutically acceptable salt or prodrug thereof or its use as further described herein is provided.

In another embodiment of the present invention, a β-D or β-L nucleoside of the formula:

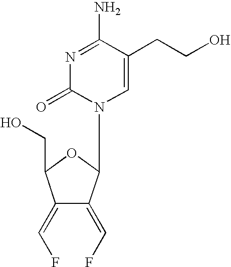

or its pharmaceutically acceptable salt or prodrug thereof or its use as further described herein is provided.

In another embodiment of the present invention, a β-D or β-L nucleoside of the formula:

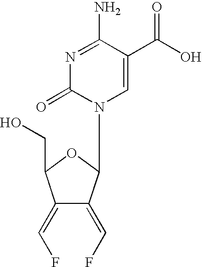

or its pharmaceutically acceptable salt or prodrug thereof or its use as further described herein is provided.

In another embodiment of the present invention, a β-D or β-L nucleoside of the formula:

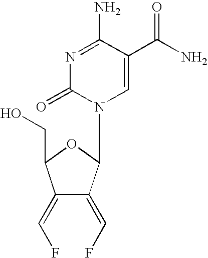

or its pharmaceutically acceptable salt or prodrug thereof or its use as further described herein is provided.

In another embodiment of the present invention, a β-D or β-L nucleoside of the formula:

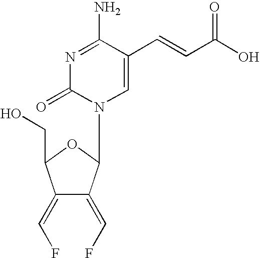

or its pharmaceutically acceptable salt or prodrug thereof or its use as further described herein is provided.

In another embodiment of the present invention, a β-D or β-L nucleoside of the formula:

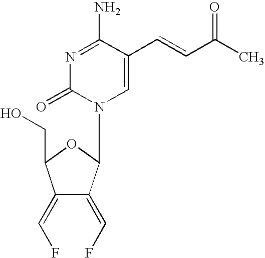

or its pharmaceutically acceptable salt or prodrug thereof or its use as further described herein is provided.

In one embodiment of the invention, the nucleoside of the invention is the isolated β-D or β-L isomer. In another embodiment of the invention, the nucleosides are enantiomerically enriched. In yet another embodiment of the invention, the nucleosides is in a enantiomeric mixture in which the desired enantiomer is at least 95%, 98% or 99% pure or free of its corresponding enantiomer.

III. Stereoisomerism and Polymorphism

Compounds of the present invention have at least two chiral centers, and may exist in and be isolated in optically active and racemic forms. Some compounds may exhibit polymorphism. The present invention encompasses racemic, optically-active, polymorphic, or stereoisomeric form, or mixtures thereof, of a compound of the invention, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution.

Optically active forms of the compounds can be prepared using any method known in the art, including by resolution of the racemic form by recrystallization techniques, by synthesis from optically-active starting materials, by chiral synthesis, or by chromatographic separation using a chiral stationary phase.

Examples of methods to obtain optically active materials include at least the following.

i) physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization—a technique whereby the individual enantiomers are separately crystallized from a solution of the racemate, possible only if the latter is a conglomerate in the solid state;

iii) enzymatic resolutions—a technique whereby partial or complete separation of a racemate by virtue of differing rates of reaction for the enantiomers with an enzyme;

iv) enzymatic asymmetric synthesis—a synthetic technique whereby at least one step of the synthesis uses an enzymatic reaction to obtain an enantiomerically pure or enriched synthetic precursor of the desired enantiomer;

v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;

vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors—a synthetic technique whereby the desired enantiomer is obtained from non-chiral starting materials and where the stereochemical integrity is not or is only minimally compromised over the course of the synthesis;

x) chiral liquid chromatography—a technique whereby the enantiomers of a racemate are separated in a liquid mobile phase by virtue of their differing interactions with a stationary phase (including via chiral HPLC). The stationary phase can be made of chiral material or the mobile phase can contain an additional chiral material to provoke the differing interactions;

xi) chiral gas chromatography—a technique whereby the racemate is volatilized and enantiomers are separated by virtue of their differing interactions in the gaseous mobile phase with a column containing a fixed non-racemic chiral adsorbent phase;

xii) extraction with chiral solvents—a technique whereby the enantiomers are separated by virtue of preferential dissolution of one enantiomer into a particular chiral solvent;

xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

Chiral chromatography, including simulated moving bed chromatography, is used in one embodiment. A wide variety of chiral stationary phases are now commercially available.

IV. Definitions

The term “alkyl,” as used herein, unless otherwise specified, refers to a saturated straight, branched, or cyclic, primary, secondary, or tertiary hydrocarbon, including but not limited to those of C₁ to C₁₆, and specifically includes methyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. The alkyl group can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, azido, thiol, imine, sulfonic acid, sulfate, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphate, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference. Alkyl specifically includes CF₃, CH₂CF₃, and CF₂CF₃.

In the text, whenever the term C(alkyl range) is used, the term independently includes each member of that class as if specifically and separately set out. As a nonlimiting example, the term “C₁₋₆” independently represents each species that falls within the scope. Alkyl groups include, but are not limited to the radicals of methane, ethane, propane, cyclopropane, 2-methylpropane (isobutane), n-butane, 2,2-dimethylpropane (neopentane), cytobutane, 1,1 dimethylcyclopropane, 2-methylbutane, trans-1,2-dimethylcyclopropane, ethylcyclopropane, n-pentane, methylcyclobutane, cis-1,2-dimethylcyclopropane, spiropentane, cyclopentane, 2,2-dimethylbutane, 1,1,2-trimethylcyclopropane, 2,3-dimethylbutane, 2-methylpentane, 3-methylpentane, 1,2,3-trimethylcyclopropane, n-hexane, ethylcyclobutane, methylcyclopentane, 2,2dimethylpentane, 2,4-dimethylpentane, cyclohexane, 2,2,3-trimethylbutane, 3,3-dimethylpentane, 1,1-dimethylcyclopentane, 2,3-dimethylpentane, 2-methylhexane, trans-1,3-dimethylcyclopentane, cis-1,3-dimethylcyclopentane, 3-methylhexane, trans-1,2-dimethylcyclopentane, 3-ethylpentane, quadricyclane (quadricyclo [2,2,1,0^{2.6},0^{3.5}] heptane), n-heptane, 2,2,4-trimethylpentane, cis-1,2-dimethylcyclopentane, methylcyclohexane, ethylcyclopentane, 1,1,3-trimethylcyclopentane, 2,2-dimethylhexane, 2,5-dimethylhexane, 1,trans-2,cis-4trimethylcyclopentane, 2,4-dimethylhexane, 2,2,3-trimethylpentane, 1,trans-2,cis-3-trimethylcyclopentane, 3,3-dimethylhexane, 2,3,4-trimethylpentane, 1,1,2-trimethylcyclopentane, 2,3,3-trimethylpentane, 2,3-dimethylhexane, 3-ethyl-2-methylpentane, 1,cis-2,trans-4-trimethylcyclopentane, 1,cis-2,trans-3trimethylcyclopentane, 2-methylheptane, 4-methylheptane, 3,4-dimethylhexane, 1,cis-2,cis-4trimethylcyclopentane, 3-ethyl-3-methylpentane, 3-ethylhexane, 3-methylheptane, cylotheptane (suberane), trans-1,4-dimethylcyclohexane, 1,1-dimethylcyclohexane, cis-1,3-dimethylcychohexane, trans-1-ethyl-3-methylcyclopentane, trans-1-ethyl-2-methylcyclopentane, cis-1-ethyl-3-methylcyclopentane, 1-ethyl-1-methylcyclopentane, 2,2,4,4-tetramethylpentane, 1,cis-2-cis-3-trimethylcyclopentane, trans-1,2-dimethylcyclohexane, 2,2,5-trimethylhexane, trans-1,3-dimethylcyclohexane, n-octane, isopropylcyclopentane, 2,2,4-trimethylhexane, cis-1-ethyl-2-methylcyclopentane, cis-1,2-dimethylcyclohexane, 2,4,4-trimethylhexane, n-propylcyclopentane, 2,3,5-trimethylhexane, ethylcyclohexane, 2,2-dimethylheptane, 2,2,3,4-tetramethylpentane, 2,4-dimethylheptane, methylcycloheptane, 2,2,3-trimethylhexane, 4-ethyl-2-methylhexane, 3-ethyl-2.2-dimethylpentane, 4,4-dimethylheptane, 2,6-dimethylheptane, 2,5-dimethylheptane, 3,5-dimethylheptane, bicyclo[4.2.0]octane, cis-bicyclo[3.3.0]octane, 2,4-dimethyl-3-ethylpentane, 1,1,3-trimethylcyclohexane, 3,3-dimethylheptane, 2,2,5,5-tetramethylhexane, 2,3,3-trimethylhexane, 3-ethyl-2-methylhexane, trans-1,3,5-trimethylcyclohexane, 2,3,4-trimethylhexane, cis-1,3,5-trimethylcyclohexane, trans-1,2,4-trimethylcyclohexane, 2,2,3,3-tetramethylpentane, 4-ethyl-3-methylhexane, 3,3,4-trimethylhexane, 2,3-dimethylheptane, 3,4-dimethylheptane, 3-ethyl-3-methylhexane, 4-ethylheptane, 2,3,3,4-tetramethylpentane, 2,3-dimethyl-3-ethylpentane, trans-1,2,3-trimethylcyclohexane, 1-isopropyl-e-methylcyclopentane (pulegan), 4-methyloctane, 1-isopropyl-2-methylcyclopentane. It is understood to those of ordinary skill in the art that the relevant alkyl radical is named by replacing the suffix “-ane” with the suffix “-yl”.

The term “lower alkyl,” as used herein, and unless otherwise specified, refers to a C₁ to C₄ saturated straight, branched, or if appropriate, a cyclic (for example, cyclopropyl) alkyl group, including both substituted and unsubstituted forms.

The term “alkylene” or “alkenyl” refers to a saturated hydrocarbyldiyl radical of straight or branched configuration, including but not limited to those that have from one to ten carbon atoms. Included within the scope of this term are methylene, 1,2-ethane-diyl, 1,1-ethane-diyl, 1,3-propane-diyl, 1,2-propane-diyl, 1,3-butane-diyl, 1,4-butane-diyl and the like. The alkylene group or other divalent moiety disclosed herein can be optionally substituted with one or more moieties selected from the group consisting of alkyl, halo, haloalkyl, hydroxyl, carboxyl, acyl, acyloxy, amino, amido, carboxyl derivatives, alkylamino, azido, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “aryl,” as used herein, and unless otherwise specified, refers to phenyl, biphenyl, or naphthyl, and preferably phenyl. The term includes both substituted and unsubstituted moieties. The aryl group can be substituted with one or more moieties selected from the group consisting of bromo, chloro, fluoro, iodo, hydroxyl, azido, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term “aralkyl,” as used herein, and unless otherwise specified, refers to an aryl group as defined above linked to the molecule through an alkyl group as defined above. The term “alkaryl” or “alkylaryl” as used herein, and unless otherwise specified, refers to an alkyl group as defined above linked to the molecule through an aryl group as defined above. In each of these groups, the alkyl group can be optionally substituted as describe above and the aryl group can be optionally substituted with one or more moieties selected from the group consisting of alkyl, halo, haloalkyl, hydroxyl, carboxyl, acyl, acyloxy, amino, amido, azido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference. Specifically included within the scope of the term aryl are phenyl; naphthyl; phenylmethyl; phenylethyl; 3,4,5-trihydroxyphenyl; 3,4,5-trimethoxyphenyl; 3,4,5-triethoxy-phenyl; 4-chlorophenyl; 4-methylphenyl; 3,5-di-tertiarybutyl-4-hydroxyphenyl; 4-fluorophenyl; 4-chloro-1-naphthyl; 2-methyl-1-naphthylmethyl; 2-naphthylmethyl; 4-chlorophenylmethyl; 4-t-butylphenyl; 4-t-butylphenylmethyl and the like.

The term “alkylamino” or “arylamino” refers to an amino group that has one or two alkyl or aryl substituents, respectively.

The term “halogen,” as used herein, includes fluorine, chlorine, bromine and iodine.

The term “enantiomerically enriched” is used throughout the specification to describe a nucleoside which includes at least about 95%, preferably at least 96%, more preferably at least 97%, even more preferably, at least 98%, and even more preferably at least about 99% or more of a single enantiomer of that nucleoside. In a preferred embodiment, the nucleoside is an enantiomerically enriched nucleoside.

The term “host,” as used herein, refers to a unicellular or multicellular organism in which the virus can replicate, including cell lines and animals, and preferably a human. Alternatively, the host can be carrying a part of the viral genome, whose replication or function can be altered by the compounds of the present invention. The term host specifically refers to infected cells, cells transfected with all or part of the viral genome and animals, in particular, primates (including chimpanzees) and humans. Relative to abnormal cellular proliferation, the term “host” refers to unicellular or multicellular organism in which abnormal cellular proliferation can be mimicked. The term host specifically refers to cells that abnormally proliferate, either from natural or unnatural causes (for example, from genetic mutation or genetic engineering, respectively), and animals, in particular, primates (including chimpanzees) and humans. In most animal applications of the present invention, the host is a human patient. Veterinary applications, in certain indications, however, are clearly anticipated by the present invention (such as bovine viral diarrhea virus in cattle, hog cholera virus in pigs, and border disease virus in sheep).

The term “pharmaceutically acceptable salt or prodrug” is used throughout the specification to describe any pharmaceutically acceptable form (such as an ester, phosphate ester, salt of an ester or a related group) of a compound which, upon administration to a patient, provides the active compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. The compounds of this invention either possess antiviral activity against Flaviviridae viruses or anti-proliferative activity against abnormal cellular proliferation, or are metabolized to a compound that exhibits such activity.

V. Pharmaceutically Acceptable Salts and Prodrugs

In cases where compounds are sufficiently basic or acidic to form stable nontoxic acid or base salts, administration of the compound as a pharmaceutically acceptable salt may be appropriate. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. In particular, examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Any of the nucleosides described herein can be administered as a nucleotide prodrug to increase the activity, bioavailability, stability or otherwise alter the properties of the nucleoside. A number of nucleotide prodrug ligands are known. In general, alkylation, acylation or other lipophilic modification of the mono, di or triphosphate of the nucleoside will increase the stability of the nucleotide. Examples of substituent groups that can replace one or more hydrogens on the phosphate moiety are alkyl, aryl, steroids, carbohydrates, including sugars, 1,2-diacylglycerol and alcohols. Many are described in R. Jones and N. Bischofberger, Antiviral Research, 27 (1995) 1-17. Any of these can be used in combination with the disclosed nucleosides to achieve a desired effect.

The active nucleoside can also be provided as a 5′-phosphoether lipid or a 5′-ether lipid, as disclosed in the following references, which are incorporated by reference herein: Kucera, L. S., N. Iyer, E. Leake, A. Raben, Modest E. K., D. L. W., and C. Piantadosi. 1990. “Novel membrane-interactive ether lipid analogs that inhibit infectious HIV-1 production and induce defective virus formation.” AIDS Res. Hum. Retro Viruses. 6:491-501; Piantadosi, C., J. Marasco C. J., S. L. Morris-Natschke, K. L. Meyer, F. Gumus, J. R. Surles, K. S. Ishaq, L. S. Kucera, N. Iyer, C. A. Wallen, S. Piantadosi, and E. J. Modest. 1991. “Synthesis and evaluation of novel ether lipid nucleoside conjugates for anti-HIV activity.” J. Med. Chem. 34:1408.1414; Hosteller, K. Y., D. D. Richman, D. A. Carson, L. M. Stuhmiller, G. M. T. van Wijk, and H. van den Bosch. 1992. “Greatly enhanced inhibition of human immunodeficiency virus type 1 replication in CEM and HT4-6C cells by 3′-deoxythymidine diphosphate dimyristoylglycerol, a lipid prodrug of 3,-deoxythymidine.” Antimicrob. Agents Chemother. 36:2025.2029; Hosetler, K. Y., L. M. Stuhmiller, H. B. Lenting, H. van den Bosch, and D. D. Richman, 1990. “Synthesis and antiretroviral activity of phospholipid analogs of azidothymidine and other antiviral nucleosides.” J. Biol. Chem. 265:61127.

Nonlimiting examples of U.S. patents that disclose suitable lipophilic substituents that can be covalently incorporated into the nucleoside, preferably at the 5′-OH position of the nucleoside or lipophilic preparations, include U.S. Pat. No. 5,149,794 (Sep. 22, 1992, Yatvin et al.); U.S. Pat. No. 5,194,654 (Mar. 16, 1993, Hostetler et al., U.S. Pat. No. 5,223,263 (Jun. 29, 1993, Hostetler et al.); U.S. Pat. No. 5,256,641 (Oct. 26, 1993, Yatvin et al.); U.S. Pat. No. 5,411,947 (May 2, 1995, Hostetler et al.); U.S. Pat. No. 5,463,092 (Oct. 31, 1995, Hostetler et al.); U.S. Pat. No. 5,543,389 (Aug. 6, 1996, Yatvin et al.); U.S. Pat. No. 5,543,390 (Aug. 6, 1996, Yatvin et al.); U.S. Pat. No. 5,543,391 (Aug. 6, 1996, Yatvin et al.); and U.S. Pat. No. 5,554,728 (Sep. 10, 1996; Basava et al.), all of which are incorporated herein by reference. Foreign patent applications that disclose lipophilic substituents that can be attached to the nucleosides of the present invention, or lipophilic preparations, include WO 89/02733, WO 90/00555, WO 91/16920, WO 91/18914, WO 93/00910, WO 94/26273, WO 96/15132, EP 0 350 287, EP 93917054.4, and WO 91/19721.

VI. Pharmaceutical Compositions

Pharmaceutical compositions based upon a β-D or β-L compound of formula (I)-(XX) or its pharmaceutically acceptable salt or prodrug can be prepared in a therapeutically effective amount for treating a Flaviviridae virus or abnormal cellular proliferation, in combination with a pharmaceutically acceptable additive, carrier or excipient. The therapeutically effective amount may vary with the infection or condition to be treated, its severity, the treatment regimen to be employed, the pharmacokinetics of the agent used, as well as the patient treated.

In one aspect according to the present invention, the compound according to the present invention is formulated preferably in admixture with a pharmaceutically acceptable carrier. In general, it is preferable to administer the pharmaceutical composition in orally administrable form, but formulations may be administered via parenteral, intravenous, intramuscular, transdermal, buccal, subcutaneous, suppository or other route. Intravenous and intramuscular formulations are preferably administered in sterile saline. One of ordinary skill in the art may modify the formulation within the teachings of the specification to provide numerous formulations for a particular route of administration without rendering the compositions of the present invention unstable or compromising its therapeutic activity. In particular, a modification of a desired compound to render it more soluble in water or other vehicle, for example, may be easily accomplished by routine modification (salt formulation, esterification, etc.).

In certain pharmaceutical dosage forms, the prodrug form of the compound, especially including acylated (acetylated or other) and ether derivatives, phosphate esters and various salt forms of the present compounds, is preferred. One of ordinary skill in the art will recognize how to readily modify the present compound to a prodrug form to facilitate delivery of active compound to a targeted site within the host organism or patient. The artisan also will take advantage of favorable pharmacokinetic parameters of the prodrug form, where applicable, in delivering the desired compound to a targeted site within the host organism or patient to maximize the intended effect of the compound in the treatment of Flaviviridae (including HCV) infections or conditions related to abnormal cellular proliferation.

The amount of compound included within therapeutically active formulations, according to the present invention, is an effective amount for treating the infection or condition, in preferred embodiments, a Flaviviridae (including HCV) infection or a condition related to abnormal cellular proliferation. In general, a therapeutically effective amount of the present compound in pharmaceutical dosage form usually ranges from about 0.1 mg/kg to about 100 mg/kg or more, depending upon the compound used, the condition or infection treated and the route of administration. For purposes of the present invention, a prophylactically or preventively effective amount of the compositions, according to the present invention, falls within the same concentration range as set forth above for therapeutically effective amount and is usually the same as a therapeutically effective amount.

Administration of the active compound may range from continuous (intravenous drip) to several oral administrations per day (for example, Q.I.D., B.I.D., etc.) and may include oral, topical, parenteral, intramuscular, intravenous, subcutaneous, transdermal (which may include a penetration enhancement agent), buccal and suppository administration, among other routes of administration. Enteric-coated oral tablets may also be used to enhance bioavailability and stability of the compounds from an oral route of administration. The most effective dosage form will depend upon the pharmacokinetics of the particular agent chosen, as well as the severity of disease in the patient. Oral dosage forms are particularly preferred, because of ease of administration and prospective favorable patient compliance.

To prepare the pharmaceutical compositions according to the present invention, a therapeutically effective amount of one or more of the compounds according to the present invention is preferably mixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated for sustained release by standard techniques. The use of these dosage forms may significantly impact the bioavailability of the compounds in the patient.

For parenteral formulations, the carrier will usually comprise sterile water or aqueous sodium chloride solution, though other ingredients, including those that aid dispersion, also may be included. Where sterile water is to be used and maintained as sterile, the compositions and carriers must also be sterilized. Injectable suspensions may also be prepared, in which case appropriate liquid carriers, suspending agents and the like may be employed.

Liposomal suspensions (including liposomes targeted to viral antigens) may also be prepared by conventional methods to produce pharmaceutically acceptable carriers. This may be appropriate for the delivery of free nucleosides, acyl nucleosides or phosphate ester prodrug forms of the nucleoside compounds according to the present invention.

In particularly preferred embodiments according to the present invention, the compounds and compositions are used to treat, prevent or delay the onset of Flaviviridae (including HCV) infections or conditions related to abnormal cellular proliferation. Preferably, to treat, prevent or delay the onset of the infection or condition, the compositions will be administered in oral dosage form in amounts ranging from about 250 micrograms, more typically at least 10, 25, 50, 100, 250, 300, 500 milligram, up to about 1 gram or more at least once a day, preferably, or up to four times a day. The present compounds are preferably administered orally, but may be administered parenterally, topically or in suppository form.

The compounds according to the present invention may be advantageously employed prophylactically to prevent Flaviviridae (including HCV) infections or conditions related to abnormal cellular proliferation or to prevent the occurrence of clinical symptoms associated with the viral infection or condition. Thus, the present invention also encompasses methods for the prophylactic treatment of viral infection, and in particular Flaviviridae (including HCV) infections or of a condition related to abnormal cellular proliferation. In this aspect, according to the present invention, the present compositions are used to prevent or delay the onset of a Flaviviridae (including HCV) infection or a condition related to abnormal cellular proliferation. This prophylactic method comprises administration to a patient in need of such treatment, or who is at risk for the development of the virus or condition, an amount of a compound according to the present invention effective for alleviating, preventing or delaying the onset of the viral infection or condition. In the prophylactic treatment according to the present invention, it is preferred that the antiviral or antiproliferative compound utilized should be low in toxicity and preferably non-toxic to the patient. It is particularly preferred in this aspect of the present invention that the compound that is used should be maximally effective against the virus or condition and should exhibit a minimum of toxicity to the patient. In the case of Flaviviridae (including HCV) infections or conditions related to abnormal cellular proliferation, compounds according to the present invention, which may be used to treat these disease states, may be administered within the same dosage range for therapeutic treatment (i.e., about 250 micrograms up to 1 gram or more from one to four times per day for an oral dosage form) as a prophylactic agent to prevent the proliferation of a Flaviviridae (including HCV) infections or conditions related to abnormal cellular proliferation, or alternatively, to prolong the onset of a Flaviviridae (including HCV) infections or conditions related to abnormal cellular proliferation, which manifests itself in clinical symptoms.

In addition, compounds according to the present invention can be administered in combination or alternation with one or more antiviral, anti-HBV, anti-HCV or anti-herpetic agent or interferon, anti-cancer or antibacterial agents, including other compounds of the present invention. Certain compounds according to the present invention may be effective for enhancing the biological activity of certain agents according to the present invention by reducing the metabolism, catabolism or inactivation of other compounds and as such, are co-administered for this intended effect.

This invention is further illustrated in the following sections. The Experimental Details section and Examples contained therein are set forth to aid in an understanding of the invention. This section is not intended to, and should not be interpreted to, limit in any way the invention set forth in the claims that follow thereafter.

VII. Therapies for the Treatment of Flaviviridae Infection

It has been recognized that drug-resistant variants of viruses can emerge after prolonged treatment with an antiviral agent. Drug resistance most typically occurs by mutation of a gene that encodes for an enzyme used in the viral replication cycle, and most typically in the case of HCV, the RNA-dependent-RNA polymerase. It has been demonstrated that the efficacy of a drug against viral infection can be prolonged, augmented, or restored by administering the compound in combination or alternation with a second, and perhaps third, antiviral compound that induces a different mutation from that caused by the principle drug. Alternatively, the pharmacokinetics, biodistribution or other parameter of the drug can be altered by such combination or alternation therapy. In general, combination therapy is typically preferred over alternation therapy because it induces multiple simultaneous stresses on the virus.

Examples of agents that have been identified as active against the hepatitis C virus, and thus can be used in combination or alternation with one or more nucleosides of general formula (I)-(XX) include those described in the following numbered paragraphs.

(1) interferon and/or ribavirin.

(2) Substrate-based NS3 protease inhibitors (Attwood et al., Antiviral peptide derivatives, PCT WO 98/22496, 1998; Attwood et al., Antiviral Chemistry and Chemotherapy 1999, 10, 259-273; Attwood et al., Preparation and use of amino acid derivatives as anti-viral agents, German Patent Pub. DE 19914474; Tung et al. Inhibitors of serine proteases, particularly hepatitis C virus NS3 protease, PCT WO 98/17679), including alphaketoamides and hydrazinoureas, and inhibitors that terminate in an electrophile such as a boronic acid or phosphonate (Llinas-Brunet et al, Hepatitis C inhibitor peptide analogues, PCT WO 99/07734).

(3) Non-substrate-based inhibitors such as 2,4,6-trihydroxy-3-nitro-benzamide derivatives (Sudo K. et al., Biochemical and Biophysical Research Communications, 1997, 238, 643-647; Sudo K. et al. Antiviral Chemistry and Chemotherapy, 1998, 9, 186), including RD3-4082 and RD3-4078, the former substituted on the amide with a 14 carbon chain and the latter processing apara-phenoxyphenyl group.

(4) Thiazolidine derivatives which show relevant inhibition in a reverse-phase HPLC assay with an NS3/4A fusion protein and NS5A/5B substrate (Sudo K. et al., Antiviral Research, 1996, 32, 9-18), especially compound RD-1-6250, possessing a fused cinnamoyl moiety substituted with a long alkyl chain, RD4 6205 and RD4 6193.

(5) Thiazolidines and benzanilides identified in Kakiuchi N. et al. J. EBS Letters 421, 217-220; Takeshita N. et al. Analytical Biochemistry, 1997, 247, 242-246.

(6) A phenan-threnequinone possessing activity against protease in a SDS-PAGE and autoradiography assay isolated from the fermentation culture broth of Streptomyces sp., Sch 68631 (Chu M. et al., Tetrahedron Letters, 1996, 37, 7229-7232), and Sch 351633, isolated from the fungus Penicillium griscofuluum, which demonstrates activity in a scintillation proximity assay (Chu M. et al., Bioorganic and Medicinal Chemistry Letters 9, 1949-1952).

(7) Selective NS3 inhibitors based on the macromolecule elgin c, isolated from leech (Qasim M. A. et al., Biochemistry, 1997, 36, 1598-1607).

(8) Helicase inhibitors (Diana G. D. et al., Compounds, compositions and methods for treatment of hepatitis C, U.S. Pat. No. 5,633,358; Diana G. D. et al., Piperidine derivatives, pharmaceutical compositions thereof and their use in the treatment of hepatitis C, PCT WO 97/36554).

(9) Polymerase inhibitors such as nucleotide analogues, gliotoxin (Ferrari R. et al. Journal of Virology, 1999, 73, 1649-1654), and the natural product cerulenin (Lohmann V. et al., Virology, 1998, 249, 108-118).

(10) Antisense phosphorothioate oligodeoxynucleotides (S-ODN) complementary to sequence stretches in the 5′ non-coding region (NCR) of the virus (Alt M. et al., Hepatology, 1995, 22, 707-717), or nucleotides 326-348 comprising the 3′ end of the NCR and nucleotides 371-388 located in the core coding region of the HCV RNA (Alt M. et al., Archives of Virology, 1997, 142, 589-599; Galderisi U. et al., Journal of Cellular Physiology, 1999,181, 251-257).

(11) Inhibitors of IRES-dependent translation (Ikeda N et al., Agent for the prevention and treatment of hepatitis C, Japanese Patent Pub. JP-08268890; Kai Y. et al. Prevention and treatment of viral diseases, Japanese Patent Pub. JP-10101591).

(12) Nuclease-resistant ribozymes (Maccjak, D. J. et al., Hepatology 1999, 30, abstract 995).

(13) Nucleoside analogs have also been developed for the treatment of Flaviviridae infections.

(14) Idenix Pharmaceuticals, Ltd. discloses branched nucleosides, and their use in the treatment of HCV and flaviviruses and pestiviruses in International Publication Nos. WO 01/90121 (filed May 23, 2001) and WO 01/92282 (filed May 26, 2001). A method for the treatment of hepatitis C infection (and flaviviruses and pestiviruses) in humans and other host animals is disclosed in the Idenix publications that includes administering an effective amount of a biologically active 1′, 2′, 3′ or 4′-branched β-D or β-L nucleosides or a pharmaceutically acceptable salt or prodrug thereof, administered either alone or in combination, optionally in a pharmaceutically acceptable carrier.

(15) WO 01/96353 (filed Jun. 15, 2001) to Indenix Pharmaceuticals, Ltd. discloses 3′-prodrugs of 2′-deoxy-β-L-nucleosides for the treatment of HBV. U.S. Pat. No. 4,957,924 to Beauchamp discloses various therapeutic esters of acyclovir.

(16) Other patent applications disclosing the use of certain nucleoside analogs to treat hepatitis C virus include: PCT/CA00/01316 (WO 01/32153; filed Nov. 3, 2000) and PCT/CA01/00197 (WO 01/60315; filed Feb. 19, 2001) filed by BioChem Pharma, Inc. (now Shire Biochem, Inc.); PCT/US02/01531 (WO 02/057425; filed Jan. 18, 2002) and PCT/US02/03086 (WO 02/057287; filed Jan. 18, 2002) filed by Merck & Co., Inc., PCT/EP01/09633 (WO 02/18404; published Aug. 21, 2001) filed by Roche, and PCT Publication No. WO 01/79246 (filed Apr. 13, 2001) and WO 02/32920 (filed Oct. 18, 2001) by Pharmasset.

(17) Other miscellaneous compounds including 1-amino-alkylcyclohexanes (U.S. Pat. No. 6,034,134 to Gold et al.), alkyl lipids (U.S. Pat. No. 5,922,757 to Chojkier et al.), vitamin E and other antioxidants (U.S. Pat. No. 5,922,757 to Chojkier et al.), squalene, amantadine, bile acids (U.S. Pat. No. 5,846,964 to Ozeki et al.), N-(phosphonoacetyl)-L-aspartic acid, (U.S. Pat. No. 5,830,905 to Diana et al.), benzenedicarboxamides (U.S. Pat. No. 5,633,388 to Diana et al.), polyadenylic acid derivatives (U.S. Pat. No. 5,496,546 to Wang et al.), 2′,3′-dideoxyinosine (U.S. Pat. No. 5,026,687 to Yarchoan et al.), and benzimidazoles (U.S. Pat. No. 5,891,874 to Colacino et al.).

(18) Other compounds currently in clinical development for treatment of hepatitis c virus include: Interleukin-10 by Schering-Plough, IP-501 by Interneuron, Merimebodib VX-497 by Vertex, AMANTADINE (Symmetrel) by Endo Labs Solvay, HEPTAZYME by RPI, IDN-6556 by Idun Pharma., XTL-002 by XTL., HCV/MF59 by Chiron, CIVACIR by NABI, LEVOVIRIN by ICN, VIRAMIDINE by ICN, ZADAXIN (thymosin alfa-1) by Sci Clone, CEPLENE (histamine dihydrochloride) by Maxim, VX 950/LY 570310 by Vertex/Eli Lilly, ISIS 14803 by Isis Pharmaceutical/Elan, IDN-6556 by Idun Pharmaceuticals, Inc. and JTK 003 by AKROS Pharma.

(19) U.S. Pat. No. 6,348,587 to Emory University and the University of Georgia Research Foundation discloses the use of 2′-fluoronucleosides for the treatment of HIV, hepatitis B, hepatitis C and abnormal cellular proliferation.

VIII. Therapies for the Treatment of Abnormal Cellular Proliferation

Examples of agents that have been identified as active against abnormal cellular proliferation, and thus can be used in combination or alternation with one or more β-D or β-L-nucleosides of general formula (I)-(XX) include:

Alkylating Agents

Nitrogen Mustards: Mechlorethamine (Hodgkin's disease, non-Hodgkin's lymphomas), Cyclophosphamide, Ifosfamide (acute and chronic lymphocytic leukemias, Hodgkin's disease, non-Hodgkin's lymphomas, multiple myeloma, neuroblastoma, breast, ovary, lung, Wilms' tumor, cervix, testis, soft-tissue sarcomas), Melphalan (L-sarcolysin) (multiple myeloma, breast, ovary), Chlorambucil (chronic lymphoctic leukemia, primary macroglobulinemia, Hodgkin's disease, non-Hodgkin's lymphomas).

Ethylenimines and Methylmelamines: Hexamethylmelamine (ovary), Thiotepa (bladder, breast, ovary).

Alkyl Sulfonates: Busulfan (chronic granuloytic leukemia).

Nitrosoureas: Carmustine (BCNU) (Hodgkin's disease, non-Hodgkin's lymphomas, primary brain tumors, multiple myeloma, malignant melanoma), Lomustine (CCNU) (Hodgkin's disease, non-Hodgkin's lymphomas, primary brain tumors, small-cell lung), Semustine (methyl-CCNU) (primary brain tumors, stomach, colon), Streptozocin (STR) (malignant pancreatic insulinoma, malignant carcinoin).

Triazenes: Dacarbazine (DTIC; dimethyltriazenoimidazole-carboxamide) (malignant melanoma, Hodgkin's disease, soft-tissue sarcomas).

Antimetabolites

Folic Acid Analogs: Methotrexate (amethopterin) (acute lymphocytic leukemia, choriocarcinoma, mycosis fungoides, breast, head and neck, lung, osteogenic sarcoma).

Pyrimidine Analogs: Fluorouracil (5-fluorouracil; 5-FU), Floxuridine (5-fluoro-deoxyuridine; FUdR) (breast, colon, stomach, pancreas, ovary, head and neck, urinary bladder, premalignant skin lesions) (topical), Cytarabine (cytosine arabinoside) (acute granulocytic and acute lymphocytic leukemias), Gemcitabine (2′,2′-difluorouridine; dFdC), tezacitabine (FMdC).

Purine Analogs and Related Inhibitors: Mercaptopurine (6-mercaptopurine; 6-MP) (acute lymphocytic, acute granulocytic and chronic granulocytic leukemia), Thioguanine (6-thioguanine: TG) (acute granulocytic, acute lymphocytic and chronic granulocytic leukemia), Pentostatin (2′-deoxycyoformycin) (hairy cell leukemia, mycosis fungoides, chronic lymphocytic leukemia).

Vinca Alkaloids: Vinblastine (VLB) (Hodgkin's disease, non-Hodgkin's lymphomas, breast, testis), Vincristine (acute lymphocytic leukemia, neuroblastoma, Wilms' tumor, rhabdomyosarcoma, Hodgkin's disease, non-Hodgkin's lymphomas, small-cell lung).

Epipodophylotoxins: Etoposide (testis, small-cell lung and other lung, breast, Hodgkin's disease, non-Hodgkin's lymphomas, acute granulocytic leukemia, Kaposi's sarcoma), Teniposide (testis, small-cell lung and other lung, breast, Hodgkin's disease, non-Hodgkin's lymphomas, acute granulocytic leukemia, Kaposi's sarcoma).

Natural Products

Antibiotics: Dactinomycin (actinonmycin D) (choriocarcinoma, Wilms' tumor rhabdomyosarcoma, testis, Kaposi's sarcoma), Daunorubicin (daunomycin; rubidomycin) (acute granulocytic and acute lymphocytic leukemias), Doxorubicin (soft tissue, osteogenic, and other sarcomas; Hodgkin's disease, non-Hodgkin's lymphomas, acute leukemias, breast, genitourinary thyroid, lung, stomach, neuroblastoma), Bleomycin (testis, head and neck, skin and esophagus lung, and genitourinary tract, Hodgkin's disease, non-Hodgkin's lymphomas), Plicamycin (mithramycin) (testis, malignant hypercalcema), Mitomycin (mitomycin C) (stomach, cervix, colon, breast, pancreas, bladder, head and neck).

Enzymes: L-Asparaginase (acute lymphocytic leukemia).

Biological Response Modifiers: Interferon-alfa (hairy cell leukemia, Kaposi's sarcoma, melanoma, carcinoid, renal cell, ovary, bladder, non Hodgkin's lymphomas, mycosis fungoides, multiple myeloma, chronic granulocytic leukemia).

Antioangiogenesis Agents

Angiostatin, Endostatin.

Hormones and Antagonists

Estrogens: Diethylstibestrol Ethinyl estradiol (breast, prostate)

Antiestrogen: Tamoxifen (breast).

Androgens: Testosterone propionate Fluxomyesterone (breast).

Antiandrogen: Flutamide (prostate).

Gonadotropin-Releasing Hormone Analog: Leuprolide (prostate).

Miscellaneous Agents

Platinum Coordination Complexes: Cisplatin (cis-DDP) Carboplatin (testis, ovary, bladder, head and neck, lung, thyroid, cervix, endometrium, neuroblastoma, osteogenic sarcoma).

Anthracenedione: Mixtozantrone (acute granulocytic leukemia, breast).

Substituted Urea: Hydroxyurea (chronic granulocytic leukemia, polycythemia vera, essential thrombocytosis, malignant melanoma).

Methylhydrazine Derivative: Procarbazine (N-methylhydrazine, MIH) (Hodgkin's disease).

Adrenocortical Suppressant: Mitotane (o,p′-DDD) (adrenal cortex), Aminoglutethimide (breast).

Adrenorticosteriods: Prednisone (acute and chronic lymphocytic leukemias, non-Hodgkin's lymphomas, Hodgkin's disease, breast).

Progestins: Hydroxprogesterone caproate, Medroxyprogesterone acetate, Megestrol acetate (endometrium, breast).

IX. Synthetic Protocol

For pyrimidine nucleosides, uridine derivative (1, Scheme 1) is the starting material, which is converted into 2,2′-anhydro derivative (2) which is treated with HF in anhydrous dioxane (Codington et al., J Org. Chem., 1964, 29, 558). The corresponding 2′-fluoro-2′-deoxyuridine derivative (3) is obtained in 40-50% yield. Modification at the 4 position in 3 can be achieved by various methods. 2′-Fluoro-2′-deoxycytidine derivatives (4, R=R′=R″=H) can be readily prepared from 3 by the well-known procedures via thiation or chlorination.

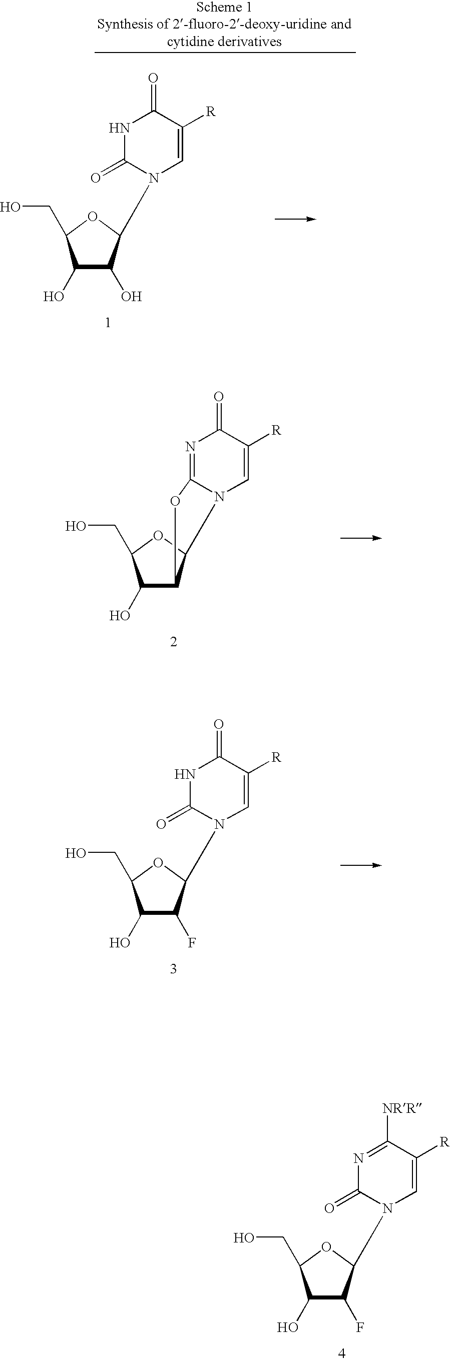

Starting from L-uridine, all the L-nucleoside counterparts synthesized in the D-series can be prepared.

One method used in the synthesis of 2′-fluoro-2′-deoxy-purine nucleosides is to start with β-D-arabinofuranosylpurines (5, Scheme 2) which is converted into 3′,5′-di-O-trityl derivatives (6) according to Pankiewiecz et al. (J. Org. chem., 1992, 57, 555 and 7315). Protected 2′-fluoro-2′-deoxyadenosine (7, X′=NHTr, Y′=H) and 2′-fluoro-2′-deoxyguanosine (7, X′=OCH₂CH₂PhNO₂, Y′=NHAc) are prepared by treatment of 6 with DAST. Mild acid treatment of 7, e.g., with trifluoroacetic acid in chloroform or methylene chloride removes the trityl group, and base treatment removes p-nitrophenetyl and N-acetyl groups to give free 2′-fluoro-2′-deoxyadenosine (8, X=NH₂, Y=H) and 2′-fluoro-2′-deoxyguanosine (X=OH, Y=NH₂). Olsen, et al., (Biochemistry, 1991, 30, 9735) synthesized 2′-fluoro-2′-deoxyadenosine using pixyl group instead of trityl protection.

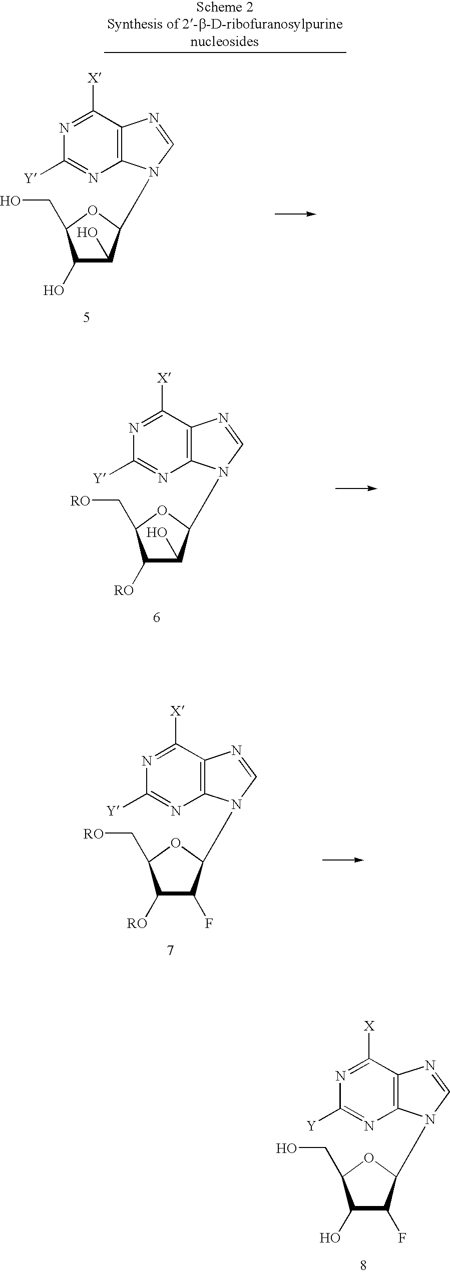

In a similar manner starting from L-adenosine or L-guanosine the enantiomers of 8 can be prepared.

gem-Difluoronucleosides can be obtained by condensation of 2,2-difluoro-1-O-acetyl-3,5-di-O-benzoyl-2-deoxo-D-ribofuranos-2-ulose (12, Scheme 3) with various silyated pyrimidine bases or with purines by the sodium salt method. The sugar can be readily prepared from 2,3-O-isopropylidene-D-glyceral (9) and ethyl bromodifluoroacetate (10) by Reformatzky reaction, followed by acidic removal of protecting groups to give lactone 11. Benzoylation of 11, and subsequent conversion of the lactone to lactol by DIBAL reduction and acetylation affords 12.

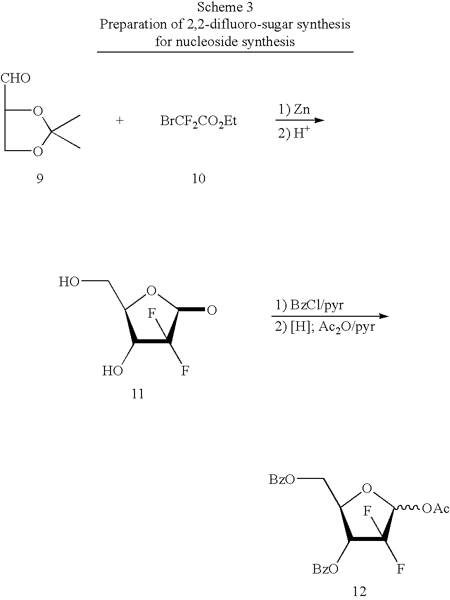

The 2′,2′-fluoromethylidene nucleosides can be synthesized from the corresponding 3′,5′-di-O-protected nucleosides (13, Scheme 4) by the procedure reported by Matthews et al. (Nucleosides Nucleotides, 1993, 12, 115) for the synthesis of (E)-2′-deoxy-2′-dehydro-2′,2′-fluoromethylidenecytidine (18, X=NH₂, R=H). Treatment of 13 (X=N=CHNMe₂ or OEt) with 1,3-dichloro-1,1,3,3-tetraisopropyldisiloxane affords the corresponding 3′,5′-O-(1,1,3,3-tetraisopropyl-1,3-disiloxane-diyl)-nucleoside 14. Swern oxidation of 14 gives the ketone 15, which, upon treatment with fluoromethyl phenylsulfone and diethyl chlorophosphate in tetrahydrofurane at −70° C., followed by lithium hexamethyldisilazane yields 16. At this point X′ can be deprotected with methanolic ammonia. Treatment of 16 with tributyltin hydride affords 17, which is converted into the corresponding free nucleoside 18 by treatment with cesium fluoride in methanol.

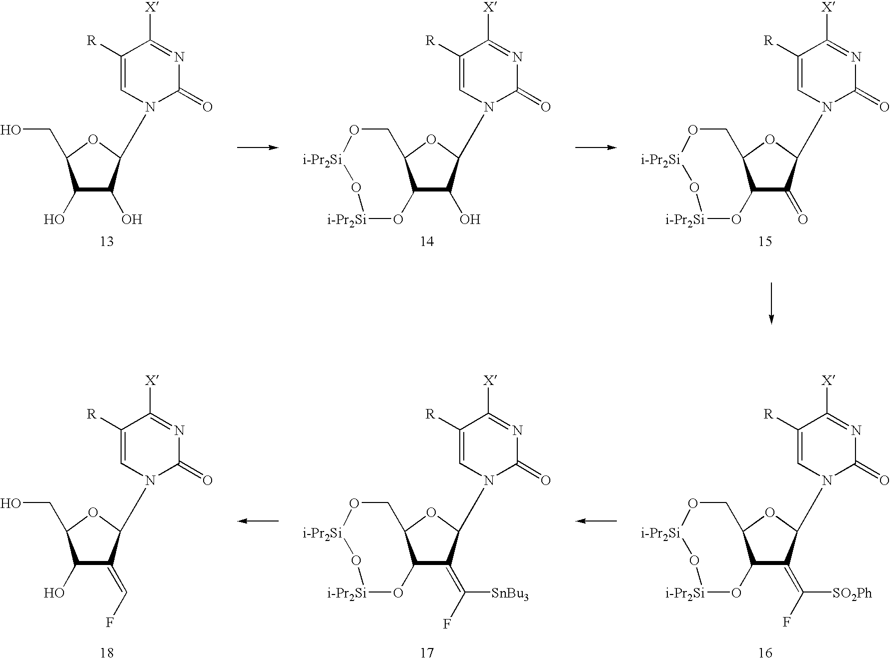

This method can be applied to various other purine and pyrimidine nucleosides. Also L-nucleoside counterparts are prepared from an L-nucleoside corresponding to 13 or its purine nucleoside analogue.

X. Biological Methods

Antiviral testing of candidate compounds for Flaviviridae: The HCV replicon system in Huh7 cells.

Huh7 cells harboring the HCV replicon can be cultivated in DMEM media (high glucose, no pyruvate) containing 10% fetal bovine serum, 1× non-essential Amino Acids, Pen-Strep-Glu (100 units/liter, 100 microgram/liter, and 2.92 mg/liter, respectively) and 500 to 1000 microgram/milliliter G418. Antiviral screening assays can be done in the same media without G418 as follows: in order to keep cells in logarithmic growth phase, seed cells in a 96-well plate at low density, for example 1000 cells per well. Add the test compound immediate after seeding the cells and incubate for a period of 3 to 7 days at 37° C. in an incubator. Media is then removed, and the cells are prepared for total nucleic acid extraction (including replicon RNA and host RNA). Replicon RNA can then be amplified in a Q-RT-PCR protocol, and quantified accordingly. The observed differences in quantification of replicon RNA is one way to express the antiviral potency of the test compound. A typical experiment demonstrates that in the negative control and in the non-active compounds-settings a comparable amount of replicon is produced. This can be concluded because the measured threshold-cycle for HCV RT-PCR in both setting is close to each other. In such experiments, one way to express the antiviral effectiveness of a compound is to subtract the threshold RT-PCR cycle of the test compound with the average threshold RT-PCR cycle of the negative control. This value is called DeltaCt (ΔCt or DCt). A ΔCt of 3.3 equals a 1-log reduction (equals EC₉₀) in replicon production. Compounds that result in a reduction of HCV replicon RNA levels of greater than 2 ΔCt values (75% reduction of replicon RNA) are candidate compounds for antiviral therapy. Such candidate compounds are belonging to structures with general formula (I)-(XX). As a positive control, recombinant interferon alfa-2a (Roferon-A, Hoffmann-Roche, New Jersey, USA) is taken alongside as positive control.

However, this HCV ΔCt value does not include any specificity parameter for the replicon encoded viral RNA-dependent RNA polymerase. In a typical setting, a compound might reduce both the host RNA polymerase activity and the replicon-encoded polymerase activity. Therefore, quantification of rRNA (or any other host RNA polymerase I product) or beta-actin mRNA (or any other host RNA polymerase II) and comparison with RNA levels of the no-drug control is a relative measurement of the effect of the test compound on host RNA polymerases.

With the availability of both the HCV ΔCt data and the rRNA ΔCt, a specificity parameter can be introduced. This parameter is obtained by subtracting both ΔCt values from each other. This results in Delta-DeltaCT values (ΔΔCt or DDCt); a value above 0 means that there is more inhibitory effect on the replicon encoded polymerase, a ΔCt value below 0 means that the host rRNA levels are more affected than the replicon levels. As a general rule, ΔCt values above 2 are considered as significantly different from the no-drug treatment control, and hence, exhibits appreciable antiviral activity. However, compounds with a ΔCt value of less than 2, but showing limited molecular cytotoxicty data (rRNA ΔCT between 0 and 2) are also possible active compounds.

In another typical setting, a compound might reduce the host RNA polymerase activity, but not the host DNA polymerase activity. Therefore, quantification of rDNA or beta-actin DNA (or any other host DNA fragment) and comparison with DNA levels of the no-drug control is a relative measurement of the inhibitory effect of the test compound on cellular DNA polymerases

With the availability of both the HCV ΔCt data and the rDNA ΔCt, a specificity parameter can be introduced. This parameter is obtained by subtracting both ΔCt values from each other. This results in ΔΔCt values; a value above 0 means that there is more inhibitory effect on the replicon encoded polymerase, a ΔΔCt value below 0 means that the host rDNA levels are more affected than the replicon levels. As a general rule, ΔΔCt values above 2 are considered as significantly different from the no-drug treatment control, and hence, is an interested compound for further evaluation. However, compounds with a ΔΔCt value of less than 2, but with limited molecular cytotoxicty (rDNA ΔCT between 0 and 2) may be desired.

Compounds that result in the specific reduction of HCV replicon RNA levels, but with limited reductions in cellular RNA and/or DNA levels are candidate compounds for antiviral therapy. Candidate compounds belonging to general formula group (I)-(XX) were evaluated for their specific capacity of reducing Flaviviridae RNA (including HCV), and potent compounds were detected.

The following working examples provide a further understanding of the method of the present invention. These examples are of illustrative purposes, and are not meant to limit the scope of the invention. Equivalent, similar or suitable solvents, reagents or reaction conditions may be substituted for those particular solvents, reagents or reaction conditions described without departing from the general scope of the method.

### EXAMPLES

Melting points were determined in open capillary tubes on an Electrothermal digit melting point apparatus and are uncorrected. The UV absorption spectra were recorded on an Uvikon 931 (KONTRON) spectrophotometer in ethanol. ¹H-NMR spectra were run at room temperature with a Varian Unity Plus 400 spectrometer. Chemical shifts are given in ppm downfield from internal tetramethylsilane as reference. Deuterium exchange, decoupling experiments or 2D-COSY were performed in order to confirm proton assignments. Signal multiplicities are represented by s (singlet), d (doublet), dd (doublet of doublets), t (triplet), q (quadruplet), br (broad), m (multiplet). All J-values are in Hz. FAB mass spectra were recorded in the positive- (FAB>0) or negative- (FAB<0) ion mode on a JEOL DX 300 mass spectrometer The matrix was 3-nitrobenzyl alcohol (NBA) or a mixture (50:50, v/v) of glycerol and thioglycerol (GT). Specific rotations were measured on a Perkin-Elmer 241 spectropolarimeter (path length 1 cm) and are given in units of 10⁻¹ deg cm² g⁻¹. Elemental analyses were performed by Atlantic Microlab Inc. (Norcross, Ga.). Analyses indicated by the symbols of the elements or functions were within ±0.4% of theoretical values. Thin layer chromatography was performed on Whatman PK5F silica gel plates, visualization of products being accomplished by UV absorbency followed by charring with 10% ethanolic sulfuric acid and heating. Column chromatography was carried out on Silica Gel (Fisher, S733-1) at atmospheric pressure.

### Example 1

### Antiviral Activity of Gemcitabine (dFdC)

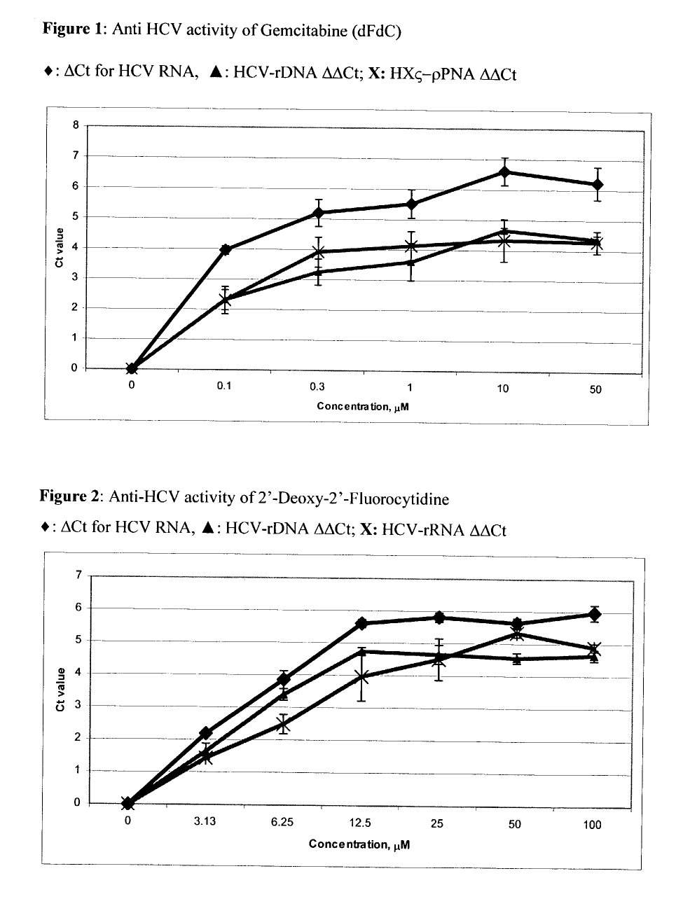

The compound was dissolved in DMSO and added to the culture media at final concentrations ranging from 0.1 to 50 μM. A 4-days incubation resulted in dose-dependant reduction of the replicon HCV RNA (FIG. 1). Since 3.3 Ct values equals 1-log reduction of replicon RNA, an EC₉₀ value was reached at approximately 70 nM. Further analysis of the reduction of cellular DNA levels (ribosomal DNA) or cellular RNA levels (ribosomal RNA) resulted in a ΔCt that expressed the inhibitory capacity of this compound on host DNA and RNA polymerases. Subtraction of these cellular ΔCt values from the antiviral ΔCt values resulted in the therapeutic index ΔΔCt values. Based on these calculations, an average EC₉₀ value, corrected for cellular toxicity, of approximately 300 nM was obtained.

### Example 2

### Antiviral Activity of 2′-Deoxy-2′F-Cytidine

The compound was dissolved in DMSO and added to the culture media at final concentrations ranging from 1 to 100 μM. A 4-days incubation resulted in dose-dependant reduction of the replicon HCV RNA (FIG. 2). Since 3.3 Ct values equals 1-log reduction of replicon RNA, an EC₉₀ value was reached at approximately 5 μM. Further analysis of the reduction of cellular DNA levels (ribosomal DNA) or cellular RNA levels (ribosomal RNA) resulted in a ΔCt that expressed the inhibitory capacity of this compound on host DNA and RNA polymerases. Subtraction of these cellular ΔCt values from the antiviral ΔCt values resulted in the therapeutic index ΔΔCt values. Based on these calculations, an average EC90 value, corrected for cellular toxicity, of approximately 10 μM was obtained.

### Example 3

### 2′-Deoxy-2′-fluorocytidine

This compound was prepared according to the method described by R. Mengel and W. Guschlbauer in Angew. Chimie Intl. Ed., 1978, 17, 525.

### Example 4

### 2′-Deoxy-2′-fluorouridine

This compound was prepared according to the method described by A. M. Kawasaki et al., in J. Med. Chem. 1993, 36, 831-841.

### Example 5

### 2′-Fluorothymidine

This compound was prepared according to the method described by A. M. Kawasaki et al., in J. Med. Chem. 1993, 36, 831-841. White crystals. ¹H-NMR (400 MHz, DMSO-d₆) δ7.80 (s, 1H, H-6), 5.91 (dd, 1H, J=2.4 & 17.6 Hz, H-1′), 5.61 (d, 1H, J=6.4 Hz, OH-3′), 5.25 (t, 1H, J=1.2 Hz, OH-5′), 5.08, 4.95 (2m, 1H, H-2′), 4.15 (m, 1H, H-3′), 3.85 (m, 1H, H-4′), 3.79, 3.60 (2m, 2H, H-5′), 1.75 (s, 3H, CH₃).

### Example 6

### 2′-Deoxy-2′-fluoro-5-methylcytidine

This compound was prepared from 2′-fluorothymidine by amination, according to the method described by K. N. Tiwari et al, in Nucleosides, Nucleotides & Nucleic Acids 2000, 19, 329-340. White crystals. ¹H-NMR (400 MHz, DMSO-d₆) δ9.33, 9.50 (2s, 2H, NH₂), 7.52 (s, 1H, H-6), 5.83 (d, 1H, J=17.2 Hz, H-1′), 5.56 (d, 1H, J=6.4 Hz, OH-3′), 5.37 (t, 1H, J=4.4 Hz, OH-5′), 4.97, 4.83 (dd, 1H, J=4.0 & 53.2 Hz, H-2′), 4.15 (m, 1H, H-3′), 3.87 (m, 1H, H-4′), 3.80, 3.60 (2m, 2H, H-5′), 1.75 (s, 3H, CH₃).

### Example 7

### 2′-Deoxy-5, 2-difluorocytidine

This compound was prepared according to the method described by L. W. Hertel et al., in J. Org. Chem. 1988, 53, 2406-2409. White crystals. ¹H-NMR (400 MHz, DMSO-d₆) δ8.24 (d, 1H, J=7.2 Hz, H-6), 7.85, 7.58 (2s, 2H, NH₂), 5.83 (d, 1H, J=12.8 Hz, H-1′), 5.57 (d, 1H, J=6.8 Hz, OH-3′), 5.36 (t, 1H, J=4.4 Hz, OH-5′), 4.94, 4.80 (2m, 1H, H-2′), 4.15 (m, 1H, H-3′), 3.87 (m, 1H, H-4′), 3.80, 3.60 (2m, 2H, H-5′).

### Example 8

### 5-Chloro-2′-deoxy-2′-fluorocytidine

This compound was prepared from 2′-deoxy-2′-fluorocytidine by chlorination, according to the method described by E. K. Ryu & J. N. Kim in Nucleosides & Nucleotides 1989, 8, 43-48. White crystals. ¹H-NMR (400 MHz, DMSO-d₆) δ8.35 (s, 1H, H-6), 7.93, 7.23 (2s, 2H, NH₂), 5.84 (d, 1H, J=16.4 Hz, H-1′), 5.56 (d, 1H, J=6.4 Hz, OH-3′), 5.37 (t, 1H, J=4.8 Hz, OH-5′), 4.96, 4.83 (dd, 1H, J=4.0 & 52.8 Hz, H-2′), 4.15 (m, 1H, H-3′), 3.88 (m, 1H, H-4′), 3.80, 3.60 (2m, 2H, H-5′).

### Example 9

### 5-Bromo-2′-deoxy-2′-fluorocytidine

This compound was prepared from 2′-deoxy-2′-fluorocytidine by bromination, according to the method described by T.-S. Lin et al., in J. Med. Chem. 1991, 34, 693-701. Pale yellow solid. ¹H-NMR (400 MHz, DMSO-d₆) δ8.42 (s, 1H, H-6), 7.93, 7.06 (2s, 2H, NH₂), 5.83 (d, 1H, J=17.2 Hz, H-1′), 5.56 (d, 1H, J=6.4 Hz, OH-3′), 5.37 (t, 1H, J=4.4 Hz, OH-5′), 4.97, 4.83 (dd, 1H, J=4.0 & 53.2 Hz, H-2′), 4.15 (m, 1H, H-3′), 3.87 (m, 1H, H-4′), 3.80, 3.60 (2m, 2H, H-5′).

### Example 10

### 2′-Deoxy-2′-fluoro-5-iodocytidine

This compound was prepared from 2′-deoxy-2′-fluorocytidine by iodination, according to the method described by T.-S. Lin et al., in J. Med. Chem. 1991, 34, 693-701. Pale yellow solid. ¹H-NMR (400 MHz, DMSO-d₆) δ8.53 (s, 1H, H-6), 7.70, 6.50 (2s, 2H, NH₂), 5.84 (d, 1H, J=17.2 Hz, H-1′), 5.56 (d, 1H, J=6.4 Hz, OH-3′), 5.37 (t, 1H, J=4.4 Hz, OH-5′), 4.97, 4.83 (dd, 1H, J=4.0 & 53.2 Hz, H-2′), 4.15 (m, 1H, H-3′), 3.86 (m, 1H, H-4′), 3.80, 3.60 (2m, 2H, H-5′).

### Example 11

### 2′-Deoxy-2′-difluorouridine (Gemcitabine, dFdC)

This compound was prepared according to the method described by L. W. Hertel et al., in J. Org. Chem. 1988, 53, 2406-2409.

### Example 12

### 2′-Deoxy-2′-difluorouridine

This compound was prepared from 2′-deoxy-2′,5-difluorocytidine by deamination, according to the method described by B. Kierdaszuk et al., in Nucleosides & Nucleotides 1999, 18, 1883-1903. White crystals. ¹H-NMR (400 MHz, DMSO-d₆) δ11.59 (br, 1H, NH), 7.79 (d, 1H, J=8.0 Hz, H-6), 6.34 (d, 1H, J=6.0 Hz, OH-3′), 6.06 (t, 1H, J=8.0 Hz, H-1′), 5.73 (d, 1H, J=8.0 Hz, H-5), 5.31 (t, 1H, J=5.2 Hz, OH-5′), 4.20 (m, 1H, H-3′), 3.85 (m, 1H, H-4′), 3.80, 3.60 (2m, 2H, H-5′).

### Example 13

### 2′-Deoxy-2′-fluoro-N4-hydroxycytidine

To a solution of 2′-deoxy-2′-fluorouridine (368 mg, 1.5 mmol) in anhydrous pyridine (10 mL) at 0° C. was added Ac₂O (612 mg, 6 mmol) dropwise. After the addition, the solution was stirred at room temp. under an argon atmosphere overnight. The solvent was evaporated to dryness in vacuo, and the residue was dissolved in CHCl₃. The organic phase was washed with saturated NaHCO₃, dried over Na₂SO₄, filtered, and concentrated in vacuo to give 436 mg (88%) of 3′,5′-di-O-acetyl-2′-deoxy-2′-fluorouridine as a white solid which was used directly for next reaction without further purification.

The above product (436 mg, 1.3 mmol) was dissolved in anhydrous acetonitrile (25 mL), and Et₃N (525 mg, 5.2 mmol) was added. The solution was cooled to 0° C., and 2,4,6-triisopropylbenzenesulfonyl chloride (813 mg, 2.6 mmol) was added, followed by 4-dimethylaminopyridine (159 mg, 1.3 mmol). The solution was stirred at room temp. under an argon atmosphere for 1 day, and then NH₂OH.HCl (185 mg, 2.6 mmol) was added. After being stirred at room temp. for another day, the solvent was evaporated, and the residue was purified by flash chromatography on silica gel eluting with CH₂Cl₂/MeOH (96:4) to give 314 mg (70%) of 3′,5′-di-O-acetyl-2′-deoxy-2′-fluoro-N⁴-hydroxycytidine as a white foam.

3′,5′-Di-O-acetyl-2′-deoxy-2′-fluoro-N⁴-hydroxycytidine (314 mg, 0.91 mmol) was suspended in 2.0 M ammonium methanol (25 mL) and stirred in a stoppered flask at room temp. for 14 h. After evaporation of the solvent, the residue was purified by flash chromatography on silica gel eluting with CH₂Cl₂/MeOH (5:1) to give 133 mg (56%) the title compound 2′-deoxy-2′-fluoro-N⁴-hydroxycytidine as white crystals. ¹H-NMR (400 MHz, DMSO-d₆) δ10.02, 9.65 (2s, 2H, NHOH), 7.06 (d, 1H, J=8.0 Hz, H-6), 5.89 (dd, 1H, J=3.2 & 17.6Hz, H-1′), 5.55-5.60 (m, 2H, H-5, OH-3′), 5.11 (t, 1H, J=4.8Hz, OH-5′), 4.97, 4.83 (dt, 1H, J=54.0 & 4.0 Hz, H-2′), 4.15 (m, 1H, H-3′), 3.80 (m, 1H, H-4′), 3.67, 3.55 (2m, 2H, H-5′).

We claim:

1. A β-D or β-L compound of the formula: 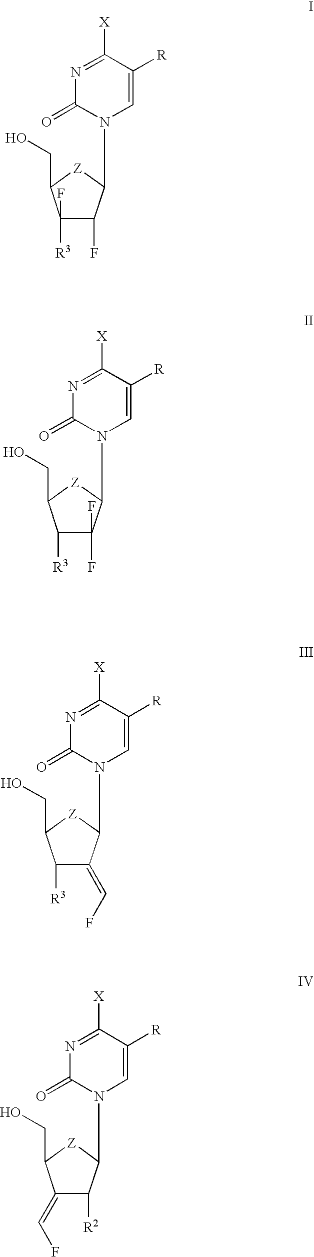 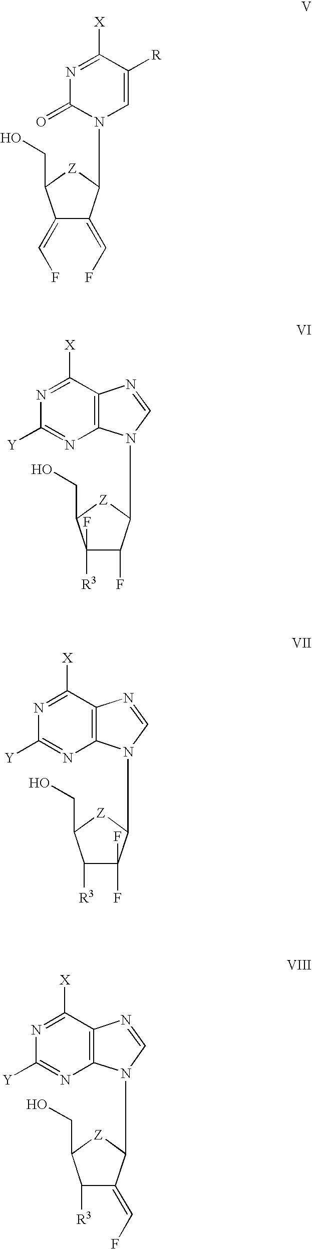 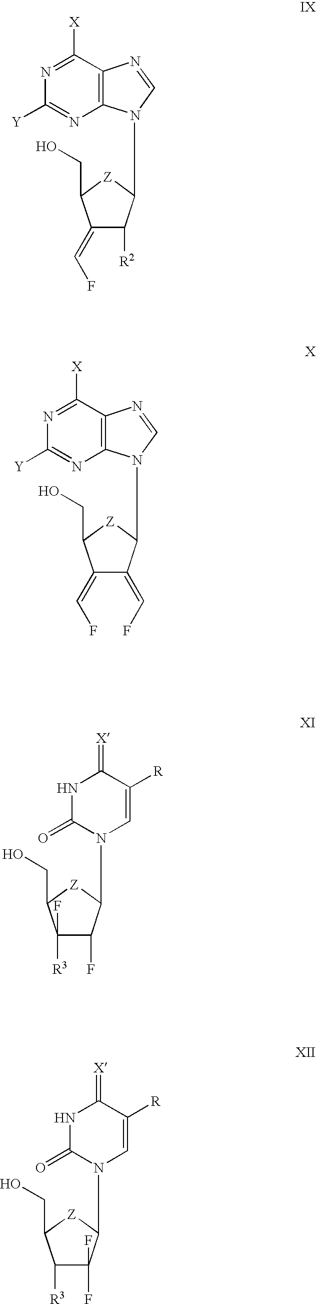 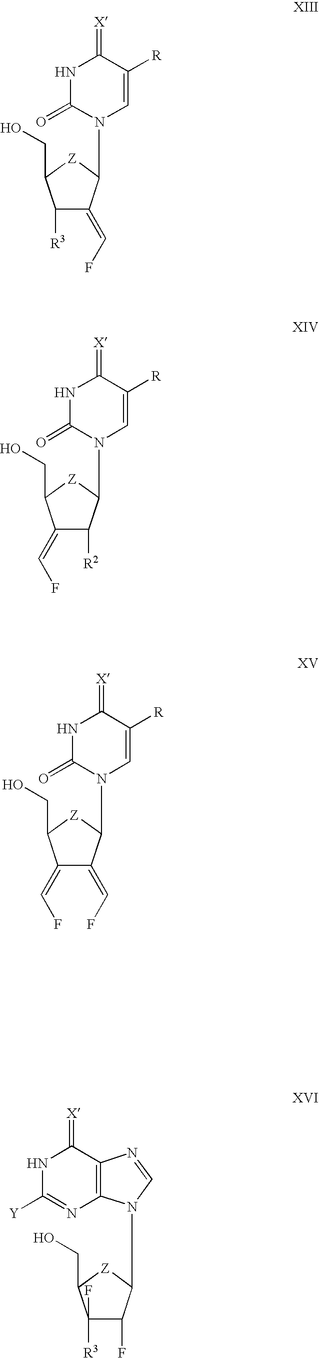 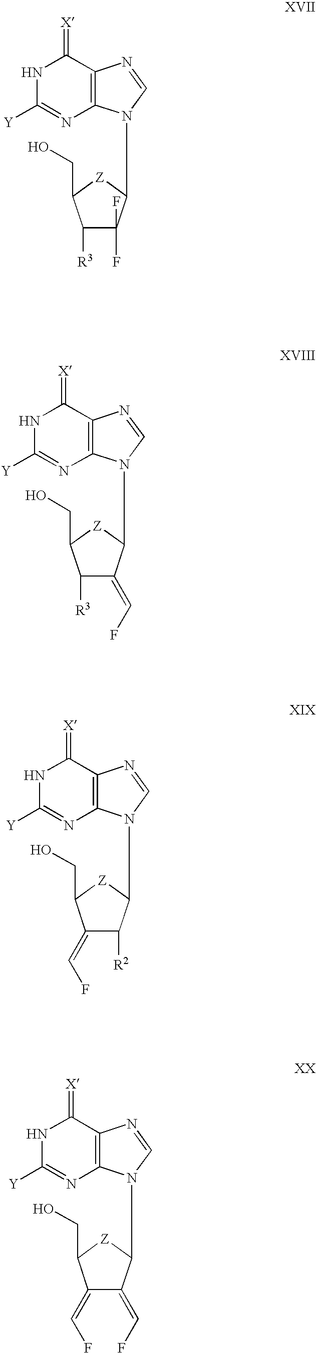
or its pharmaceutically acceptable salt or prodrug thereof, wherein:
(a) R is H, halogen (F, Cl, Br, I), OH, OR′, SH, SR′, NH₂, NHR′, NR′₂, lower alkyl of C₁-C₆, halogenated (F, Cl, Br, I) lower alkyl of C₁-C₆ such as CF₃ and CH₂CH₂F, lower alkenyl of C₂-C₆ such as CH═CH₂, halogenated (F, Cl, Br, I) lower alkenyl of C₂-C₆ such as CH═CHCl, CH═CHBr and CH═CHI, lower alkynyl of C₂-C₆ such as C≡CH, halogenated (F, Cl, Br, I) lower alkynyl of C₂-C₆, lower alkoxy of C₁-C₆ such as CH₂OH and CH₂CH₂OH, CO₂H, CO₂R′, CONH₂, CONHR′, CONR′₂, CH═CHCO₂H, CH═CHCO₂R′;
(b) X and Y are independently H, halogen, OH, OR′, OCH₃, SH, SR′, SCH₃, NH₂, NHR′, NR′₂, CH₃;
(c) each R′ is independently a hydrogen, acyl, lower alkyl of C₁-C₆ or lower cycloalkyl of C₁-C₆;
(d) Z is O, S or CH₂;
(e) R₂ is F or OH;
(f) R³ is F or OH;
(g) X′ is O, S, NH, NR′, CH₂, or CHR′;
with the proviso for compound II that when X is NH₂ or compound XII when X is NH and R is H, then R³ is not OH.

2. The β-D compound of claim 1 of the formula: 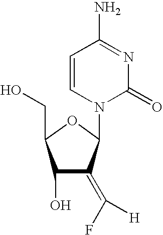
or its pharmaceutically acceptable salt or prodrug thereof.

3. The β-D compound of claim 1 of the formula: 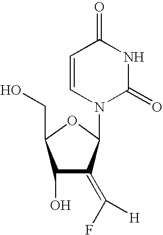
or its pharmaceutically acceptable salt or prodrug thereof.

4. The compound as described in any of the preceding claims 1-3, wherein the said compound is in combination with a pharmaceutically acceptable carrier and in the form of a dosage unit.

5. The compound as described in claim 4, wherein the dosage unit contains about 10 mg to about 1 gram of the compound.

6. The compound as described in claim 4 or 5, wherein the dosage unit is a capsule or tablet.

7. A pharmaceutical composition for the treatment or prophylaxis of a Flaviviridae infection in a host, comprising an effective amount of a compound of claim 1 in combination with a pharmaceutically acceptable carrier.

8. A pharmaceutical composition for the treatment or prophylaxis of a Flaviviridae infection in a host, comprising an effective amount of a compound of claim 1, in combination with a pharmaceutically acceptable carrier, and with another effective anti-viral agent.

9. The pharmaceutical composition according to claim 8, wherein the Flaviviridae infection is HCV.

10. A pharmaceutical composition for the treatment or prophylaxis of abnormal cellular proliferation comprising an effective amount of a compound of claim 1, in combination with a pharmaceutically acceptable carrier.

11. A pharmaceutical composition for the treatment or prophylaxis of abnormal cellular proliferation comprising an effective amount of a compound of claim 1, optionally in a pharmaceutically acceptable carrier, with another effective agent to treat abnormal cellular proliferation.

12. The pharmaceutical composition according to claim 10 or 11, wherein the abnormal cellular proliferation is a malignant tumor.

13. A pharmaceutical composition for the treatment or prophylaxis of a hepatitis C virus in a host, comprising an effective amount of a β-D compound of structure: 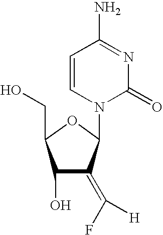
or its pharmaceutically acceptable salt or prodrug thereof, together with a pharmaceutically acceptable carrier or diluent.

14. A pharmaceutical composition for the treatment or prophylaxis of a abnormal cellular proliferation in a patient, comprising an effective amount of a β-D compound of structure: 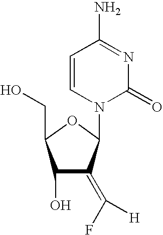
or its pharmaceutically acceptable salt or prodrug thereof, together with a pharmaceutically acceptable carrier or diluent.

15. A pharmaceutical composition for the treatment or prophylaxis of a hepatitis C virus in a host, comprising an effective amount of a β-D compound of structure: 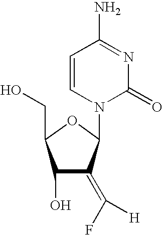
or its pharmaceutically acceptable salt or prodrug thereof, in combination with one or more other antivirally effective agents.

16. A pharmaceutical composition for the treatment or prophylaxis of a abnormal cellular proliferation in a patient, comprising an effective amount of a β-D compound of structure: 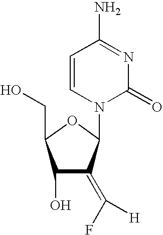
or its pharmaceutically acceptable salt or prodrug thereof, in combination with one or more other anti-abnormal cellular proliferation agents.

17. The pharmaceutical compositions according to any one of claims 7-16, wherein the composition is in the form of a dosage unit.

18. The pharmaceutical composition according to claim 17, wherein the dosage unit contains about 10 mg to about 1 gram of the compound.

19. The pharmaceutical composition according to claim 17 or 18, wherein the dosage unit is a tablet or capsule.

20. A method of treatment or prophylaxis of Flaviviridae infection is a host, comprising administering an effective amount of the compound of the formula: 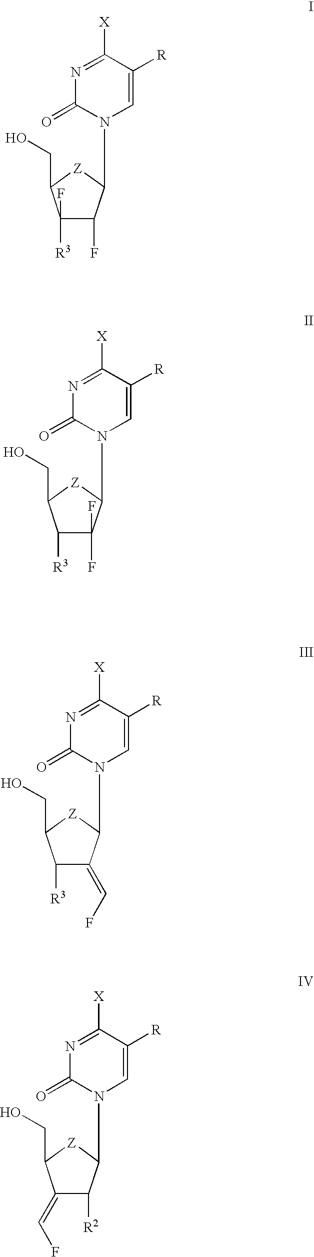 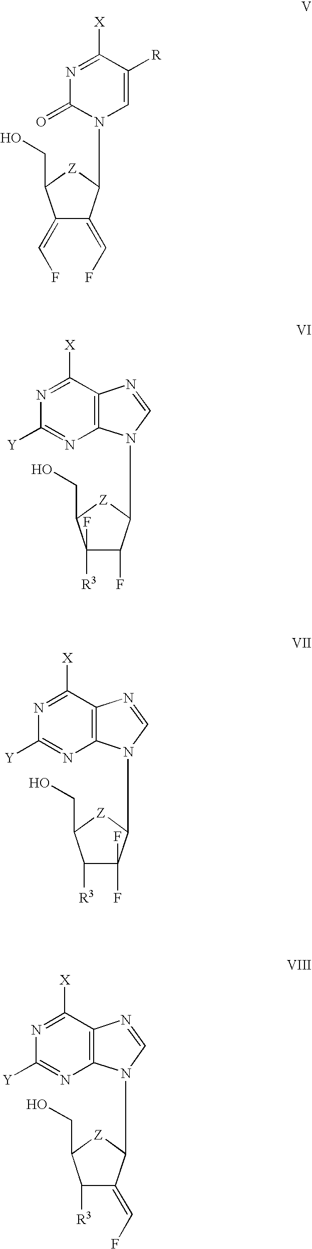 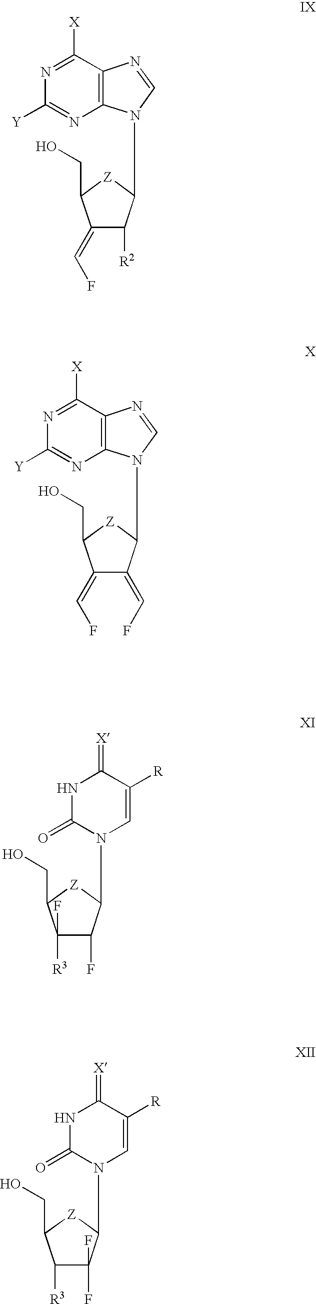 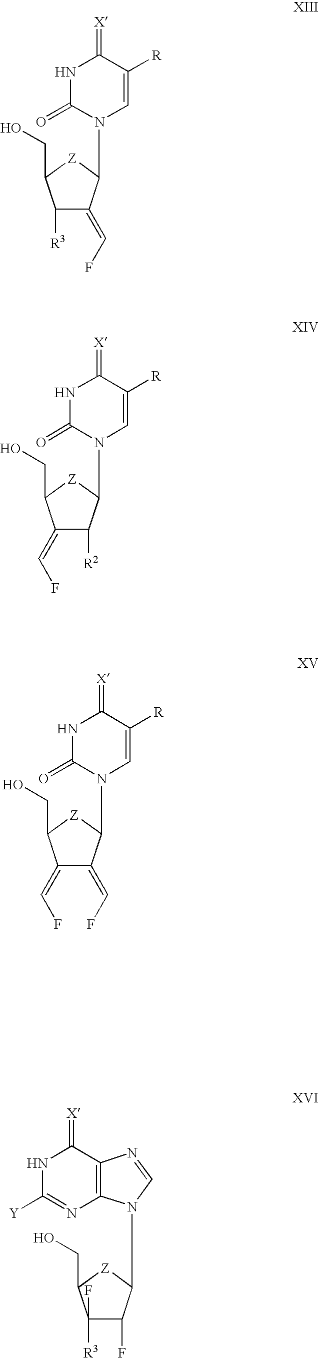 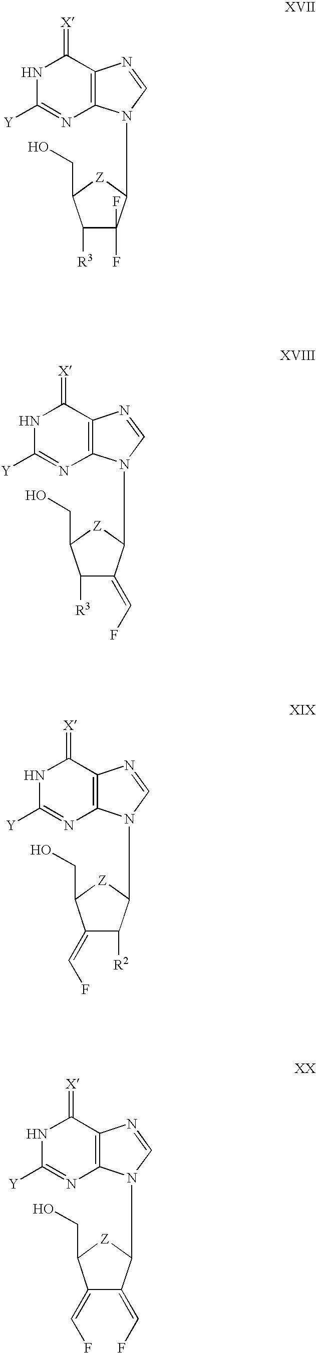
or its pharmaceutically acceptable salt or prodrug thereof, wherein:
(a) R is H, halogen (F, Cl, Br, I), OH, OR′, SH, SR′, NH₂, NHR′, NR′₂, lower alkyl of C₁-C₆, halogenated (F, Cl, Br, I) lower alkyl of C₁-C₆ such as CF₃ and CH₂CH₂F, lower alkenyl of C₂-C₆ such as CH═CH₂, halogenated (F, Cl, Br, I) lower alkenyl of C₂-C₆ such as CH═CHCl, CH═CHBr and CH═CHI, lower alkynyl of C₂-C₆ such as C≡CH, halogenated (F, Cl, Br, I) lower alkynyl of C₂-C₆, lower alkoxy of C₁-C₆ such as CH₂OH and CH₂CH₂OH, CO₂H, CO₂R′, CONH₂, CONHR′, CONR′₂, CH═CHCO₂H, CH═CHCO₂R′;
(b) X and Y are independently H, halogen, OH, OR′, OCH₃, SH, SR′, SCH₃, NH₂, NHR′, NR′₂, CH₃;
(c) each R′ is independently a hydrogen, acyl, lower alkyl of C₁-C₆ or lower cycloalkyl of C₁-C₆;
(d) Z is O, S or CH₂;
(e) R² is F or OH;
(f) R³ is F or OH;
(g) X′ is O, S, NH, NR′, CH₂, or CHR′;
with the proviso for compound II that when X is NH₂ or compound XII when X is NH and R is H, then R³ is not OH.

21. A method of treatment or prophylaxis of abnormal cellular proliferation in a patient, comprising administering an effective amount of the compound of the formula: 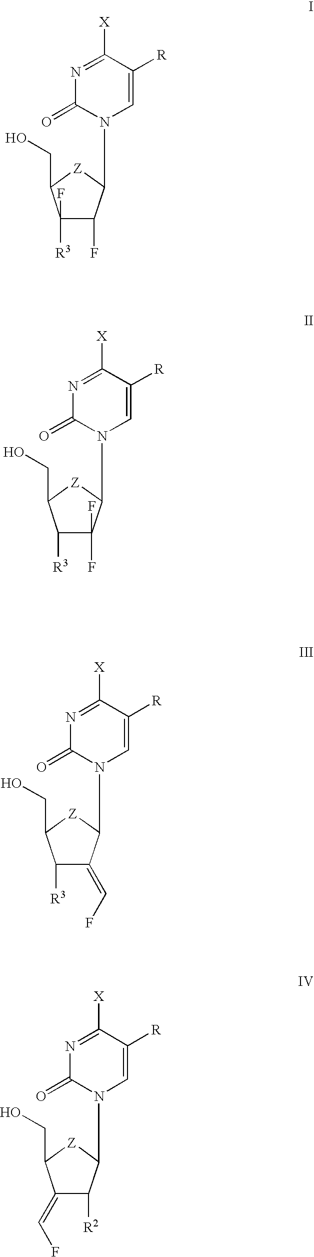 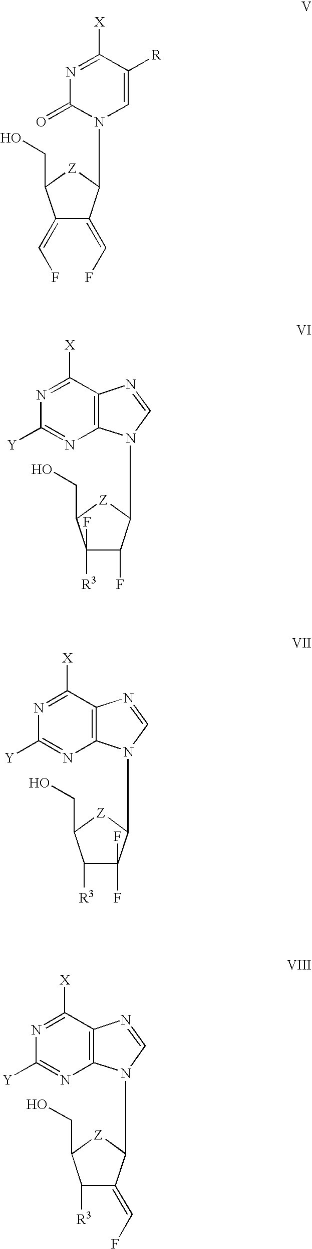 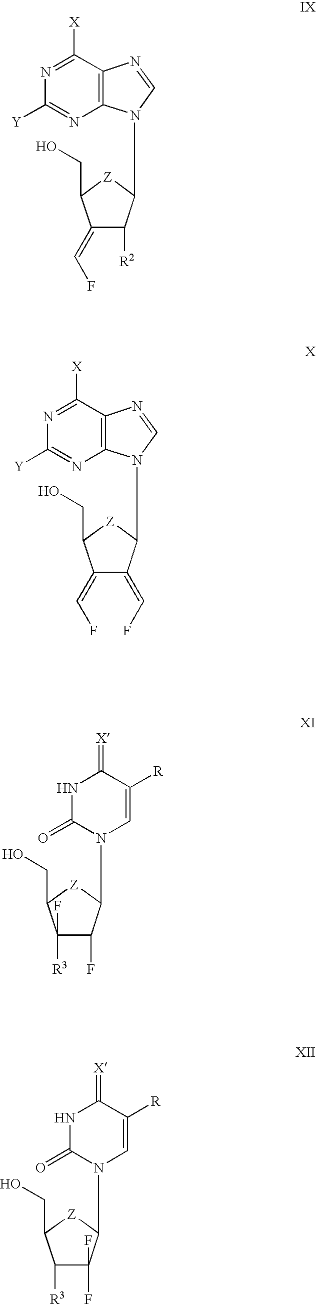 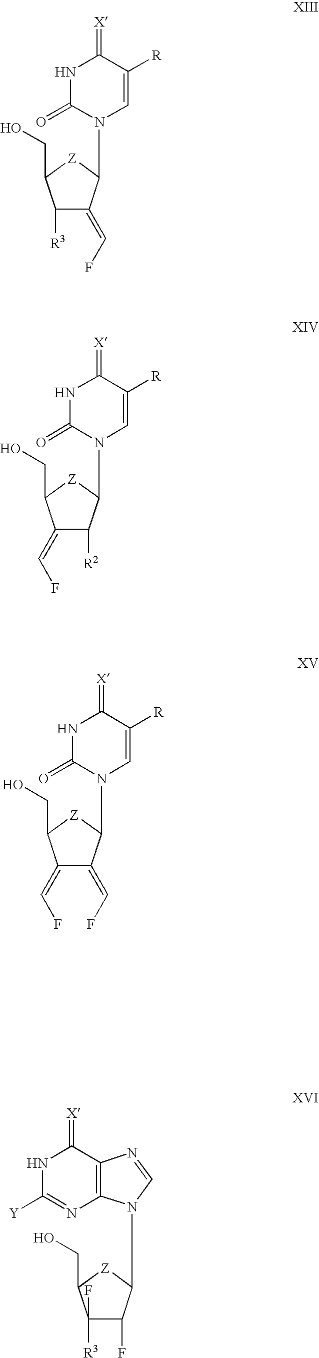 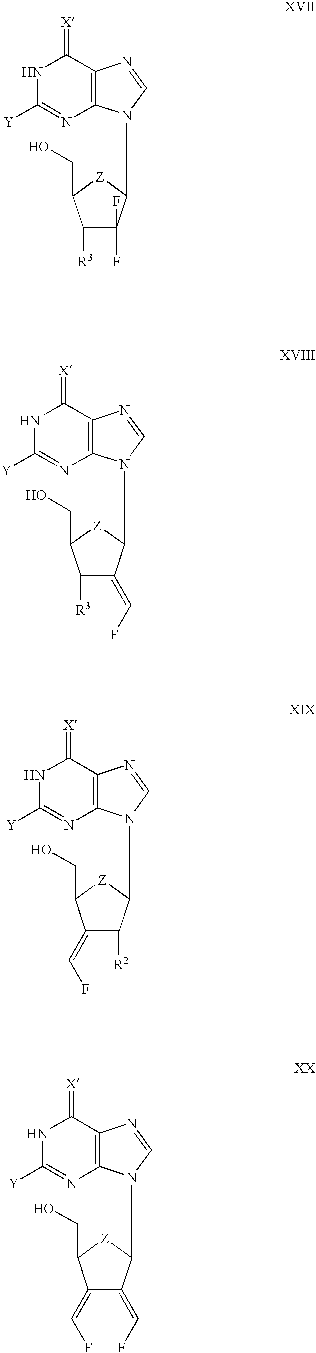
or its pharmaceutically acceptable salt or prodrug thereof, wherein:
(a) R is H, halogen (F, Cl, Br, I), OH, OR′, SH, SR′, NH₂, NHR′, NR′₂, lower alkyl of C₁-C₆, halogenated (F, Cl, Br, I) lower alkyl of C₁-C₆ such as CF₃ and CH₂CH₂F, lower alkenyl of C₂-C₆ such as CH═CH₂, halogenated (F, Cl, Br, I) lower alkenyl of C₂-C₆ such as CH═CHCl, CH═CHBr and CH═CHI, lower alkynyl of C₂-C₆ such as C≡CH, halogenated (F, Cl, Br, I) lower alkynyl of C₂-C₆, lower alkoxy of C₁-C₆ such as CH₂OH and CH₂CH₂OH, CO₂H, CO₂R′, CONH₂, CONHR′, CONR′₂, CH═CHCO₂H, CH═CHCO₂R′;
(b) X and Y are independently H, halogen, OH, OR′, OCH₃, SH, SR′, SCH₃, NH₂, NHR′, NR′₂, CH₃;
(c) each R′ is independently a hydrogen, acyl, lower alkyl of C₁-C₆ or lower cycloalkyl of C₁-C₆;
(d) Z is O, S or CH₂;
(e) R² is F or OH;
(f) R³ is F or OH;
(g) X′ is O, S, NH, NR′, CH₂, or CHR′;
with the proviso for compound II that when X is NH₂ or compound XII when X is NH and R is H, then R³ is not OH.

22. A method for the treatment or prophylaxis of Flaviviridae infection in a host, comprising an effective amount of a β-D compound of structure: 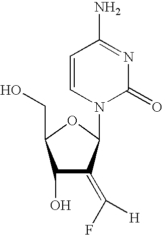
or its pharmaceutically acceptable salt or prodrug thereof .

23. A method for the treatment or prophylaxis of abnormal cellular proliferation in a patient, comprising an effective amount of a β-D compound of structure: 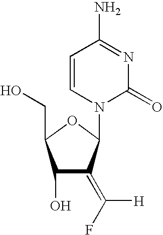
or its pharmaceutically acceptable salt or prodrug thereof.

24. The method of treatment or prophylaxis of Flaviviridae infection is a host, comprising administering an effective amount of the compound of the formula: 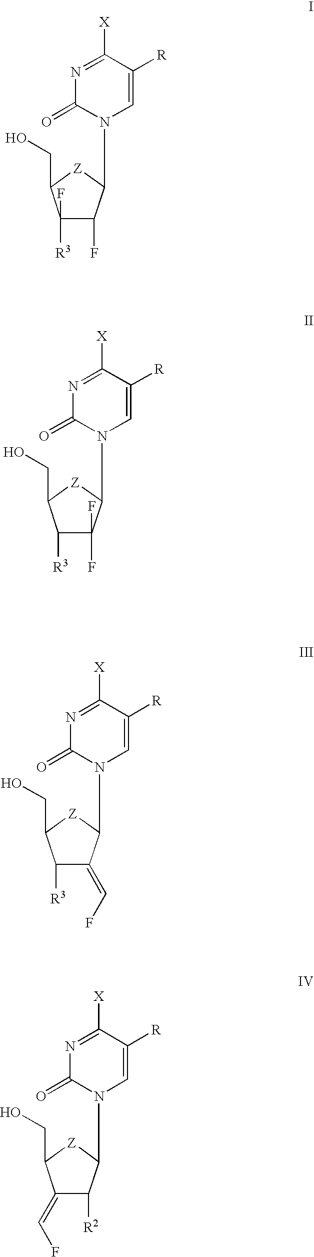 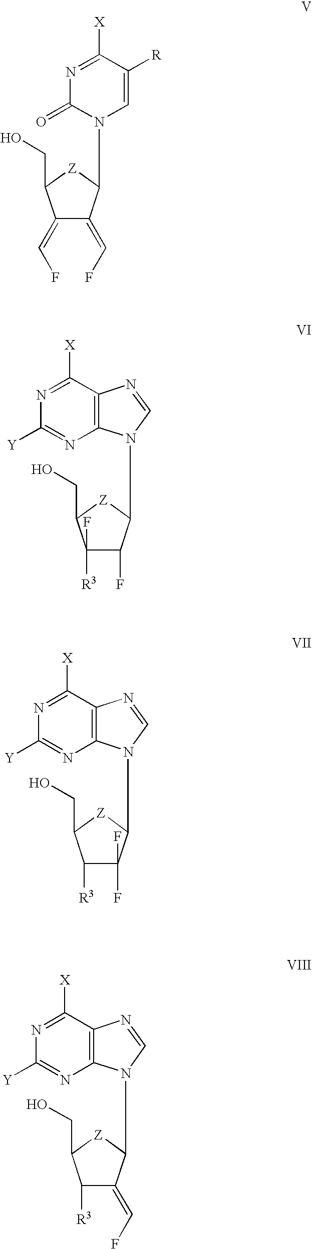 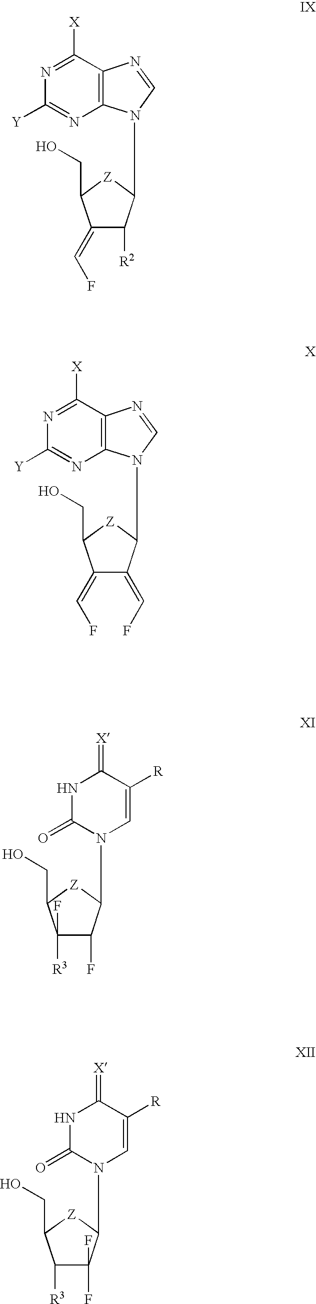 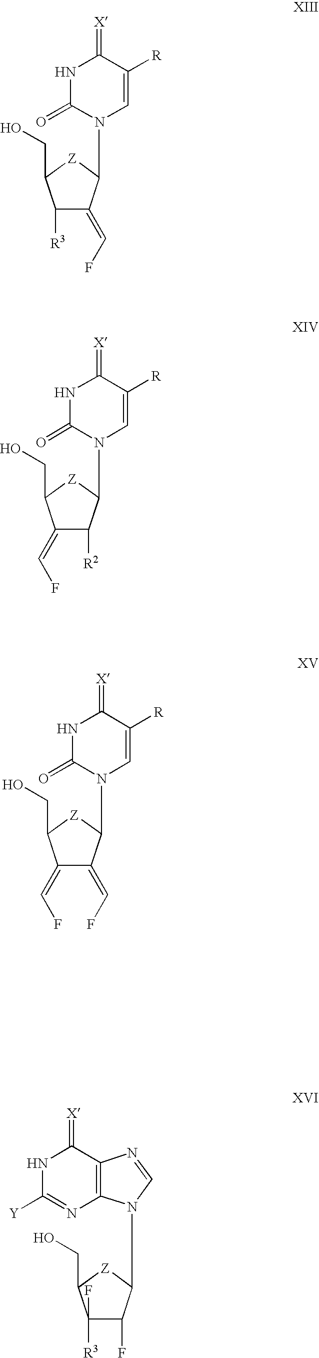 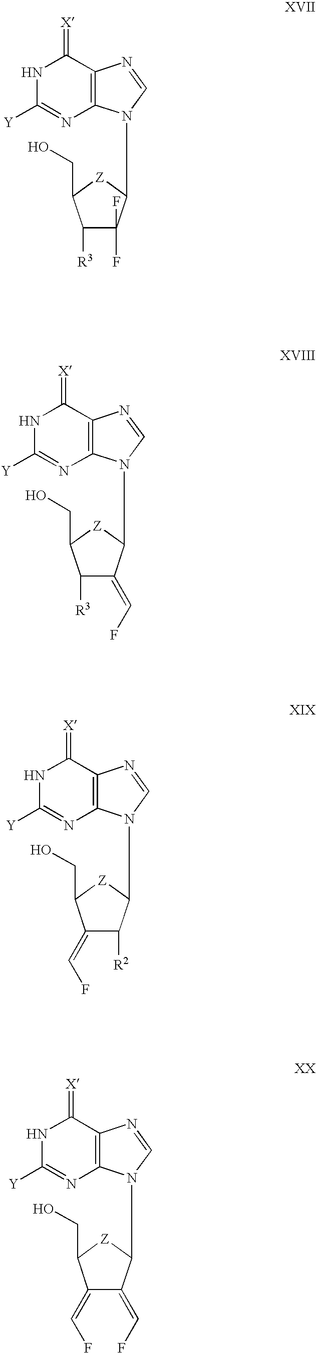
or its pharmaceutically acceptable salt or prodrug thereof, wherein:
(a) R is H, halogen (F, Cl, Br, I), OH, OR′, SH, SR′, NH₂, NHR′, NR′₂, lower alkyl of C₁-C₆, halogenated (F, Cl, Br, I) lower alkyl of C₁-C₆ such as CF₃ and CH₂CH₂F, lower alkenyl of C₂-C₆ such as CH═CH₂, halogenated (F, Cl, Br, I) lower alkenyl of C₂-C₆ such as CH═CHCl, CH═CHBr and CH═CHI, lower alkynyl of C₂-C₆ such as C≡CH, halogenated (F, Cl, Br, I) lower alkynyl of C₂-C₆, lower alkoxy of C₁-C₆ such as CH₂OH and CH₂CH₂OH, CO₂H, CO₂R′, CONH₂, CONHR′, CONR′₂, CH═CHCO₂H, CH═CHCO₂R′;
(b) X and Y are independently H, halogen, OH, OR′, OCH₃, SH, SR′, SCH₃, NH₂, NHR′, NR′₂, CH₃;
(c) each R′ is independently a hydrogen, acyl, lower alkyl of C₁-C₆ or lower cycloalkyl of C₁-C₆;
(d) Z is O, S or CH₂;
(e) R²is F or OH;
(f) R³ is F or OH;
(g) X′ is O, S, NH, NR′, CH₂, or CHR′;
with the proviso for compound II that when X is NH₂ or compound XII when X is NH and R is H, then R³ is not OH, in combination or alternation with other anti-viral agents.

25. The method of treatment or prophylaxis of abnormal cellular proliferation in a patient, comprising administering an effective amount of the compound of the formula: 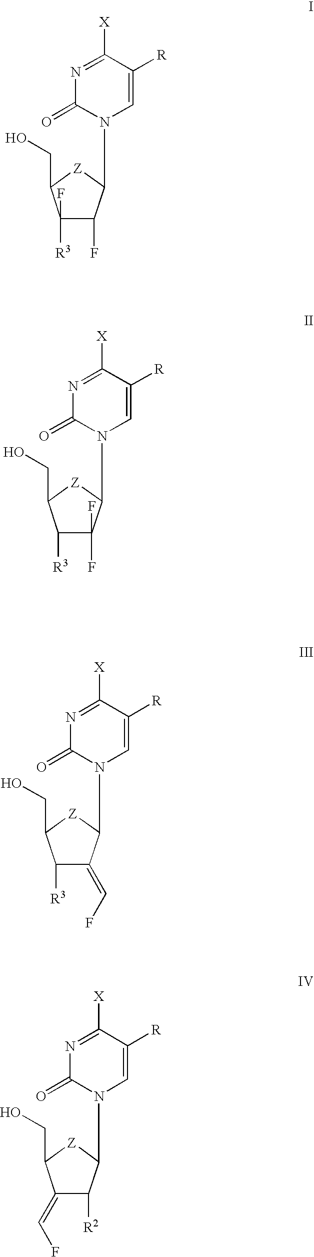 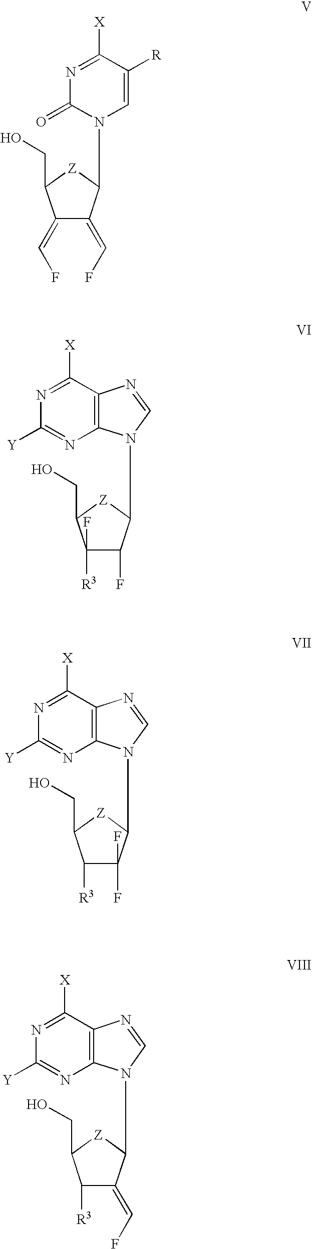 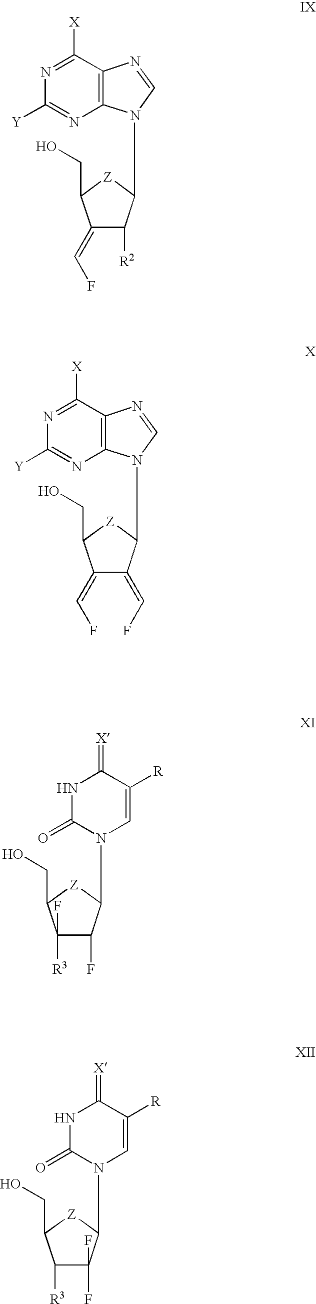 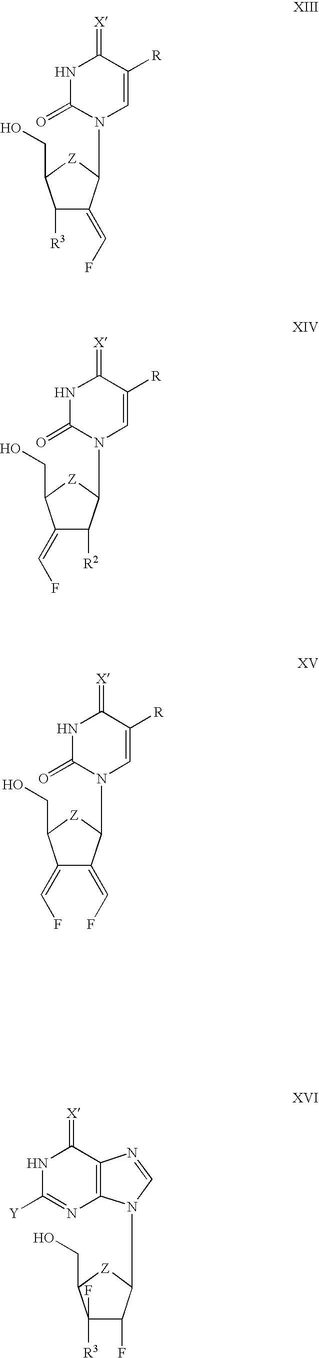 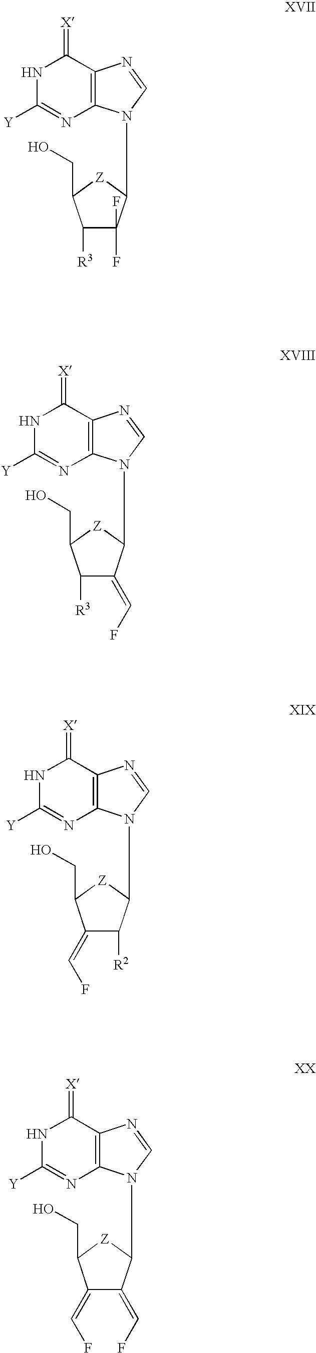
or its pharmaceutically acceptable salt or prodrug thereof, wherein:
(a) R is H, halogen (F, Cl, Br, I), OH, OR′, SH, SR′, NH₂, NHR′, NR′₂, lower alkyl of C₁-C₆, halogenated (F, Cl, Br, I) lower alkyl of C₁-C₆ such as CF₃ and CH₂CH₂F, lower alkenyl of C₂-C₆ such as CH═CH₂, halogenated (F, Cl, Br, I) lower alkenyl of C₂-C₆ such as CH═CHCl, CH═CHBr and CH═CHI, lower alkynyl of C₂-C₆ such as C≡CH, halogenated (F, Cl, Br, I) lower alkynyl of C₂-C₆, lower alkoxy of C₁-C₆ such as CH₂OH and CH₂CH₂OH, CO₂H, CO₂R′, CONH₂, CONHR′, CONR′₂, CH═CHCO₂H, CH═CHCO₂R′;
(b) X and Y are independently H, halogen, OH, OR′, OCH₃, SH, SR′, SCH₃, NH₂, NHR′, NR′₂, CH₃;
(c) each R′ is independently a hydrogen, acyl, lower alkyl of C₁-C₆ or lower cycloalkyl of C₁-C₆;
(d) Z is O, S or CH₂;
(e) R² is F or OH;
(f) R³ is F or OH;
(g) X′ is O, S, NH, NR′, CH₂, or CHR′;
with the proviso for compound II that when X is NH₂ or compound XII when X is NH and R is H, then R³ is not OH, in combination or alternation with other agents for the treatment of abnormal cellular proliferation.

26. A method for the treatment or prophylaxis of Flaviviridae infection in a host, comprising an effective amount of a β-D compound of structure: 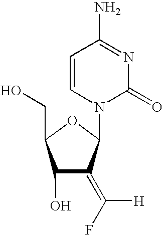
or its pharmaceutically acceptable salt or prodrug thereof, in combination or alternation with other anti-viral agents.

27. A method for the treatment or prophylaxis of abnormal cellular proliferation in a patient, comprising an effective amount of a β-D compound of structure: 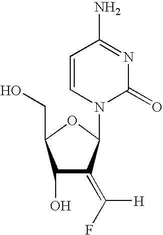
or its pharmaceutically acceptable salt or prodrug thereof, in combination or alternation with other anti-abnormal cellular proliferation agents.

28. The method according to any one of claims 20-27, wherein the compound in the form of a dosage unit.

29. The method according to claim 28, wherein the dosage unit contains about 10 mg to about 1 gram of the compound.

30. The method according to claim 28 or 29, wherein the dosage unit is a tablet or capsule.
